SEQUENCE LISTING (21 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 8 aa, Artificial Sequence
  SYNTHESIZED
IGSTENLK

SEQ ID NO: 3 - protein, 12 aa, Artificial Sequence
  SYNTHESIZED
LQTAPVPMPDLK

SEQ ID NO: 4 - protein, 6 aa, Artificial Sequence
  SYNTHESIZED
VQIINK

SEQ ID NO: 5 - protein, 9 aa, Artificial Sequence
  SYNTHESIZED
LDLSNVQSK

SEQ ID NO: 6 - protein, 19 aa, Artificial Sequence
  SYNTHESIZED
HVPGGGSVQIVYKPVDLSK

SEQ ID NO: 7 - protein, 16 aa, Artificial Sequence
  SYNTHESIZED
IGSLDNITHVPGGGNK

SEQ ID NO: 8 - protein, 15 aa, Artificial Sequence
  SYNTHESIZED
IGSLDNITHVPGGGN

SEQ ID NO: 9 - protein, 12 aa, Artificial Sequence
  SYNTHESIZED
VQIVYKPVDLSK

SEQ ID NO: 10 - protein, 10 aa, Artificial Sequence
  SYNTHESIZED
TPPSSGEPPK

SEQ ID NO: 11 - protein, 18 aa, Artificial Sequence
  SYNTHESIZED
QEFEVMEDHAGTYGLGDR

SEQ ID NO: 12 - protein, 20 aa, Artificial Sequence
  SYNTHESIZED
DQGGYTMHQDQEGDTDAGLK

SEQ ID NO: 13 - protein, 23 aa, Artificial Sequence
  SYNTHESIZED
ESPLQTPTEDGSEEPGSETSDAK

SEQ ID NO: 14 - protein, 20 aa, Artificial Sequence
  SYNTHESIZED
STPTAEDVTAPLVDEGAPGK

SEQ ID NO: 15 - protein, 39 aa, Artificial Sequence
  SYNTHESIZED
QAAAQPHTEIPEGTTAEEAGIGDTPSLEDEAAGHVTQAR

SEQ ID NO: 16 - protein, 5 aa, Artificial Sequence
  SYNTHESIZED
IATPR

SEQ ID NO: 17 - protein, 15 aa, Artificial Sequence
  SYNTHESIZED
SGYSSPGSPGTPGSR

SEQ ID NO: 18 - protein, 10 aa, Artificial Sequence
  SYNTHESIZED
TPSLPTPPTR

SEQ ID NO: 19 - protein, 5 aa, Artificial Sequence
  SYNTHESIZED
VAVVR

SEQ ID NO: 20 - protein, 10 aa, Artificial Sequence
  SYNTHESIZED
TDHGAEIVYK

SEQ ID NO: 21 - protein, 11 aa, Artificial Sequence
  SYNTHESIZED
SPVVSGDTSPR

(19) United States
(12) Patent Application Publication
(10) Pub. No.: US 2022/0299527 A1
(43) Pub. Date: Sep. 22, 2022

(54) METHODS TO DETECT MTBR TAU ISOFORMS AND USE THEREOF

(71) Applicant: Washington University, St. Louis, MO (US)

(72) Inventors: RANDALL BATEMAN, St. Louis, MO (US); CHIHIRO SATO, St. Louis, MO (US); KANTA HORIE, St Louis, MO (US); NICOLAS BARTHELEMY, St. Louis, MO (US)

(21) Appl. No.: 17/634,843

(22) PCT Filed: Aug. 13, 2020
(86) PCT No.: PCT/US20/46224

§ 371 Date: Feb. 11, 2022

Related U.S. Application Data
(60) Provisional application No. 62/886,165, filed on Aug. 13, 2019.
(60) Provisional application No. 62/970,950, filed on Feb. 6, 2020.
(60) Provisional application No. 63/044,836, filed on Jun. 26, 2020.

(51) Int. Cl. G01N 33/68; G01N 1/34

(52) U.S. Cl. CPC G01N 33/6893; G01N 33/6848; G01N 1/34; G01N 2333/4709; G01N 2800/2821

(57) ABSTRACT

The methods disclosed herein employ unique combinations of processing steps that transform a blood or CSF sample into a sample suitable for quantifying MTBR tau species, as well as other tau species. The present disclosure also encompasses the use of MTBR tau species in blood or CSF to measure pathological features and/or clinical symptoms of 3R- and 4R-tauopathies in order to diagnose, stage, and/or choose treatments appropriate for a given disease stage, and modify a given treatment regimen.

### CROSS REFERENCE TO RELATED APPLICATIONS

This application claims the priority of U.S. provisional application No. 62/886,165, filed Aug. 13, 2019, U.S. provisional application No. 62/970,950 filed Feb. 6, 2020, and U.S. provisional application No. 63/044,836, filed Jun. 26, 2020, each of which is hereby incorporated by reference in its entirety.

### GOVERNMENTAL RIGHTS

This invention was made with government support under NS095773 awarded by the National Institutes of Health. The government has certain rights in the invention.

### REFERENCE TO A SEQUENCE LISTING

This application contains a Sequence Listing that has been submitted in ASCII format via EFS-Web and is hereby incorporated by reference in its entirety. The ASCII copy, created on month day, year, is named “665135_ST25.txt”, and is 14 KB bytes in size.

### FIELD

The present disclosure encompasses methods to transform a blood or CSF sample into a sample suitable for quantifying MTBR tau species by mass spectrometry, immunoassays, or other assays known in the art. The present disclosure also encompasses the use of MTBR tau species in blood or CSF to measure pathological features and/or clinical symptoms of 3R- and 4R-tauopathies in order to diagnose, stage, and/or choose treatments appropriate for a given disease stage.

### BACKGROUND

Accumulation of tau protein as insoluble aggregates in the brain is one of the hallmarks of Alzheimer's disease and other neurodegenerative diseases called tauopathies. Tau pathology appears to propagate across brain regions and spread by the transmission of specific pathological tau species from cell to cell in a prion-like manner although the nature of these species (i.e., monomeric, oligomeric, and fibril species) and the spreading process are uncertain (Frost et al., 2009; Goedert et al., 2010, 2017; Sanders et al., 2014; Wu et al., 2016; Mirbaha et al., 2018; Lasagna-Reeves et al., 2012). Tau has six different isoforms of the full-length protein. In addition, tau has more than one hundred potential post-translational modification sites, including phosphorylation, in addition to multiple truncation sites (Meredith et al., 2013; Sato et al., 2018; Barthélemy et al., 2019; Cicognola et al., 2019; Blennow et al., 2020). Thus, identifying specific pathological tau species involved in tau spread is challenging. Several mass spectrometry (MS) studies suggest that the microtubule-binding region (MTBR) of tau is enriched in aggregates in Alzheimer's disease brain (Taniguchi-Watanabe et al., 2016; Roberts et al., 2020). Moreover, a series of cryogenic electron microscopy (Cryo-EM) studies demonstrate that the core structure of tau aggregates consists of a sub-segment of the MTBR domain and the particular conformation depends on the tauopathy (Fitzpatrick et al., 2017; Falcon et al., 2018, 2019; Zhang et al., 2020). These findings strongly suggest that MTBR tau is critical for tau aggregation. However, these studies used postmortem brain tissue. Little is known about the pathophysiology of corresponding extracellular MTBR-containing tau species in biological samples such as CSF and blood, which may serve as a surrogate biomarker of brain tau aggregates in living humans.

CSF is routinely obtained from study participants via lumbar puncture during clinical visits. Previous CSF tau biomarker studies suggested that MTBR tau was missing in CSF and focused on N-terminal and mid-domain regions (Meredith et al., 2013; Sato et al., 2018). Species composed of the N-terminus to mid-domain appear to be actively secreted from neurons into the extracellular space after truncation between the mid- and the MTBR-domain (Sato et al., 2018). Detection of MTBR tau species were reported (Barthélemy et al., 2016b, a) but have not been characterized in relationship to disease. Recently, a tau species containing a cleavage at residue 368 (tau368) within the repeat region 4 (R4) was identified in CSF (Blennow et al., 2020). It is unclear, however, whether tau368 reflects the overall pool of MTBR tau species given the variations in regions, truncations and conformational structures not captured by antibodies.

Advances in high resolution mass spectrometry techniques have created new methodologies to measure the abundance of proteins in biological samples. In spite of advances in instrumentation and data analysis software, sample preparation is still an immense challenge. The choice of sample preparation method affects the observed metabolite profile and data quality, and can ultimately affect reported results. This is particularly true for proteins and peptides in low abundance in biological samples. Peptides that fall under this umbrella include many proteolytic fragments of full length proteins, which are differentially produced in various disease processes.

Accordingly, there remains a need in the art for improved sample processing methods in order to quantify low abundance, MTBR tau species in biological fluid.

### SUMMARY

Among the various aspects of the present disclosure are provided methods to process a previously obtained biological sample in order to measure the relative or absolute concentration of tau by mass spectrometry.

One aspect of the present disclosure encompasses a method for measuring tau in a biological sample, the method comprising (a) providing a biological sample selected from a blood sample or a CSF sample; (b) removing proteins from the biological sample by protein precipitation and separation of the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography-mass spectrometry with the sample comprising proteolytic peptides of tau to detect and measure the concentration of at least one proteolytic peptide of tau.

Another aspect of the present disclosure encompasses a method for measuring tau in a biological sample, the method comprising (a) decreasing in a biological sample by affinity depletion N-terminal tau, mid-domain tau, or N-terminal tau and mid-domain tau, wherein the biological sample is a blood sample or a CSF sample; (b) removing additional proteins from the biological sample by protein precipitation and separation of the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography-mass spectrometry with the sample comprising tau peptides to detect and measure the concentration at least one proteolytic peptide of tau.

Another aspect of the present disclosure encompasses a method for measuring tau in a biological sample, the method comprising (a) decreasing in a biological sample by affinity depletion N-terminal tau, mid-domain tau, or N-terminal tau and mid-domain tau, wherein the biological sample is a blood sample or a CSF sample; (b) affinity purifying MTBR tau; (c) cleaving the purified MTBR tau with a protease and then optionally desalting the resultant cleavage product by solid phase extraction to obtain a sample comprising proteolytic peptides of MTBR tau; and (d) performing liquid chromatography-mass spectrometry with the sample comprising proteolytic peptides of MTBR tau to detect and measure the concentration at least one proteolytic peptide of MTBR tau.

Another aspect of the present disclosure encompasses a method for measuring tau in a biological sample, the method comprising (a) decreasing in a biological sample by affinity depletion N-terminal tau, mid-domain tau, or N-terminal tau and mid-domain tau, wherein the biological sample is a blood sample or a CSF sample, wherein affinity depletion comprises contacting the biological sample with an epitope binding agent that specifically binds to an epitope within amino acids 1 to 221 (inclusive), preferably within amino acids 50 to 221 (inclusive), or more preferably within amino acids 104 to 221 (inclusive) of tau-441 (or within similarly defined regions for other full-length isoforms); (b) affinity purifying MTBR tau, wherein affinity purification comprises contacting the product of step (a) with an epitope binding agent that specifically binds to an epitope that is C-terminal to the epitope recognized by the epitope binding agent of step (a); (c) cleaving the purified MTBR tau with a protease and then optionally desalting the resultant cleavage product by solid phase extraction to obtain a sample comprising proteolytic peptides of MTBR tau; and (d) performing liquid chromatography-mass spectrometry with the sample comprising proteolytic peptides of MTBR tau to detect and measure the concentration at least one proteolytic peptide of MTBR tau. In some embodiments, the epitope binding agent of step (b) specifically binds to an epitope within amino acids 221 to 441 (inclusive) of tau-441 (or within similarly defined regions for other full-length isoforms). In some embodiments, the epitope binding agent of step (b) specifically binds to an epitope within amino acids 235 to 441 (inclusive) of tau-441 (or within similarly defined regions for other full-length isoforms). In some embodiments, the epitope binding agent of step (b) specifically binds to an epitope within amino acids 235 to 368 (inclusive) of tau-441 (or within similarly defined regions for other full-length isoforms). In some embodiments, the epitope binding agent of step (b) specifically binds to an epitope within amino acids 244 to 368 (inclusive) of tau-441 (or within similarly defined regions for other full-length isoforms). In some embodiments, the epitope binding agent of step (b) specifically binds to an epitope within amino acids 244 to 299 (inclusive) of tau-441 (or within similarly defined regions for other full-length isoforms).

Prior to use in the methods disclosed herein, the biological sample may have been modified by the removal of cell debris, the addition of components (e.g., protease inhibitors, isotope labeled internal standards, detergent(s), chaotropic agent(s), etc.), and/or depletion of analytes (e.g., Aβ peptides, N-terminal tau, mid-domain tau, etc.).

Methods disclosed herein are particularly suited for measuring MTBR tau. In specific embodiments of the above, methods of the present disclosure may be used to measure the concentration of one, or more than one, tryptic peptide of tau including but not limited to IGST (SEQ ID NO: 2), VQII (SEQ ID NO: 4), LQTA (SEQ ID NO: 3), LDLS (SEQ ID NO: 5), HVPG (SEQ ID NO: 6), IGSL (SEQ ID NO: 7), and VQIV (SEQ ID NO: 9). In some examples, it may be desirable to measure the concentration of two or more tryptic peptides of tau and then calculate a ratio for the two values. As disclosed herein, ratios of HVPG (SEQ ID NO: 6) to IGSL (SEQ ID NO: 7), LQTA (SEQ ID NO: 3) to IGSL (SEQ ID NO: 7), IGST (SEQ ID NO: 2) to IGSL (SEQ ID NO: 7), VQII (SEQ ID NO: 4) to IGSL (SEQ ID NO: 7), LDLS (SEQ ID NO: 5) to IGSL (SEQ ID NO: 7), IGST (SEQ ID NO: 2) to HVPG (SEQ ID NO: 6), VQII (SEQ ID NO: 4) to HVPG (SEQ ID NO: 6), LDLS (SEQ ID NO: 5) to HVPG (SEQ ID NO: 6), and VQIV (SEQ ID NO: 7) to LDLS (SEQ ID NO: 5) may provide clinically meaningful information to diagnose tauopathies and guide treatment decisions. In still further examples, it may be desirable to determine the presence/absence of one or more additional protein and/or measure the concentration of one or more additional protein in the biological sample.

Another aspect of the present disclosure provides a method for measuring tauopathy-related pathology in a subject, the method comprising quantifying one or more mid-domain-independent MTBR tau species in a biological sample obtained from a subject, such as a blood sample or a CSF sample, wherein the amount of the quantified mid-domain-independent MTRB-tau species, or their ratios, is a representation of tauopathy-related pathology in the brain of the subject. The tauopathy may be a 3R-tauopathy, a mixed 3R/4R-tauopathy, or a 4R-tauopathy. The disease-related pathology may be tau deposition, tau post-translational modification, amyloid plaques in the brain and/or arteries of the brain, or other pathological feature known in the art. The subject may or may not have clinical symptoms of the tauopathy.

Another aspect of the present disclosure provides a method for diagnosing a tauopathy in a subject, the method comprising quantifying one or more mid-domain-independent MTBR tau species in a biological sample obtained from a subject, such as a blood sample or a CSF sample, and diagnosing a tauopathy when the quantified mid-domain-independent MTBR tau species differs/differ by about 1.5σ or more, where a is the standard deviation defined by the normal distribution measured in a control population that does not have clinical signs or symptoms of a tauopathy and is amyloid negative as measured by PET imaging and/or Aβ42/40 measurement in CSF. The tauopathy may be a 3R-tauopathy, a mixed 3R/4R-tauopathy, or a 4R-tauopathy. The subject may or may not have clinical symptoms of disease.

Another aspect of the present disclosure provides a method for measuring disease stability in a subject, the method comprising quantifying one or more mid-domain-independent MTBR tau species in a first biological sample obtained from a subject and then in a second biological sample obtained from the same subject, wherein the second biological sample was obtained after the first biological sample (e.g., after days, weeks, months, or years), and calculating the difference between the quantified MTBR tau species between the samples, wherein a statistically significant increase in the quantified MTBR tau species in the second sample indicates disease progression, a statistically significant decrease in the quantified MTBR tau species in the second sample indicates disease improvement, and no change indicates stable disease. The subject may or may not have clinical symptoms of disease.

Another aspect of the present disclosure provides a method for treating a subject with a tauopathy, the method comprising quantifying one or more mid-domain-independent MTBR tau species in a biological sample obtained from a subject, such as a blood sample or a CSF sample; and providing a treatment to the subject to improve a measurement of disease-related pathology and/or a clinical symptom, wherein the subject has a quantified MTBR tau species that differs by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF. The tauopathy may be a 3R-tauopathy, a mixed 3R/4R-tauopathy, or a 4R-tauopathy. The measurement of disease-related pathology may be tau deposition as measured by the amount of MTBR tau species and/or PET imaging, tau post-translational modification as measured by mass spectrometry or other suitable method, amyloid plaques in the brain or arteries of the brain as measured by PET imaging, amyloid plaques as measured by Aβ42/40 in CSF, or other pathological features known in the art. The clinical symptom may be dementia, as measured by a clinically validated instrument (e.g., MMSE, CDR-SB, etc.) or other clinical symptoms known in the art for 3R-, 3R/4R- and 4R-tauopathies.

These and other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1 is a schematic of the longest human tau isoform (2N4R). The N-terminus (N term), mid domain, MTBR, and C-terminus (C term) are identified for this isoform and will vary in a predictable way for other tau isoforms (e.g., 2N3R, 1NR4, 1N3R, 0N4R, and 0N3R).

FIG. 2A is a schematic illustrating several methods of the present disclosure. The method detailed within the blue box (right) is one method. The combination of the red box (left) and the blue box (right) is another method.

FIG. 2B is a schematic illustrating several methods of the present disclosure. The method detailed within the blue box (right) is one method. The combination of the red box (left) and the blue box (right) is another method.

FIG. 3A is a graph comparing the effect of three sample processing methods on the ability to quantify tau peptides from a single test sample of CSF. The test sample of CSF was not from a single individual, and disease status associated with the CSF is not available. Tau-441 peptides are identified on the x-axis and 14N/15N ratio is on the y-axis. The relative locations of the epitope recognized by the antibodies HJ8.5 and Tau1 (each depicted as a “Y”) are shown. In samples processed by the IP method (green triangles), tryptic peptides of tau from the MTBR region may be detected but have a much lower signal than tryptic peptides of tau from N-terminus to mid-domain and not quantifiable in human CSF from chronic neurodegenerative diseases including AD and healthy volunteer. In contrast, these peptides are readily detected in samples processed by the CX method (blue circle) or PostIP-CX method (red square).

FIG. 3B is an illustration depicting how sample processing may affect the population of tau proteins detected by downstream methods. In the IP method (bounded by the dashed green line), tau species with N-terminal and mid-domain epitopes recognized by antibodies (exemplified by HJ8.5 and Tau1, respectively) are immunoprecipitated. In the PostIP-CX method (bounded by the dashed red line), the tau species present after immunoprecipitation and precipitation do not have epitopes recognized by the antibodies used in the immunoprecipitation (exemplified by the “MTBR-C” illustration) or the epitopes are not accessible (exemplified by the illustrations of tau in non-linear conformations). The CX method (bounded by the dashed blue line), which occurs without prior immunoprecipitation, produces a sample with the tau species resulting from the IP method and the PostIP-CX method.

FIG. 4 is a graph of pT217% (x-axis) vs. Aβ42/40 concentration (y-axis) measured in LOAD100 and LOAD60 CSF samples. The dashed, horizontal line demarcates amyloid status defined by CSF Aβ42/40 concentration (CSF Aβ42/40>0.1389=amyloid positive, and CSF Aβ42/40<0.1389=amyloid negative). As shown, p217% correlates extremely well with amyloid status defined by this cut-off.

FIG. 5 graphically depicts the amount of two tryptic peptides of tau, TPPS and HVPG, quantified by mass spectrometry in CSF samples processed by the IP method described in Example 1 (IP_TPPS, left graph) or by the PostIP-CX method described in Examples 1 and 2 (PostIP_HVPG, right graph). The CSF samples are identified by CDR score and amyloid status. The graphic between the two graphs depicts the relative location of the tryptic peptides in tau-441. NS—not significant.

FIG. 6A graphically depicts the amount of the tryptic peptide of tau, HVPG, vs. Aβ42/40 in CSF samples processed by the PostIP-CX method described in Examples 1 and 2. The CSF samples are identified by amyloid status—amyloid positive (red) or amyloid negative (blue). The data show that measurement of HVPG in CSF samples processed by the PostIP-CX method described in Examples 1 and 2 recapitulates amyloid status in brain, as evidenced by the tight correlation with amyloid status in terms of Aβ42/40.

FIG. 6B graphically depicts the amount of the tryptic peptide of tau, HVPG, vs. pT217% in CSF samples processed by the PostIP-CX method described in Examples 1 and 2. The CSF samples are identified by amyloid status—amyloid positive (red) or amyloid negative (blue). The data show that measurement of HVPG in CSF samples processed by the PostIP-CX method described in Examples 1 and 2 recapitulates amyloid status in brain, as evidenced by the tight correlation with amyloid status in terms of pT217%.

FIG. 7A, FIG. 7B, and FIG. 7C graphically depict the amount of three tryptic peptides of tau, LQTA (FIG. 7A), HVPG (FIG. 7B), and IGSL (FIG. 7C), in CSF samples processed by the PostIP-CX method described in Examples 1 and 2. The CSF samples are grouped by CDR score and amyloid status. The data show LQTA increased in amyloid positive subjects as compared to amyloid negative subjects, even in symptomatic stages; HVPG increased in amyloid positive subjects as compared to amyloid negative subjects, especially in the asymptomatic stage; and IGSL increased in amyloid positive subjects as compared to amyloid negative subjects and decreased after the symptomatic stage.

FIG. 8A, FIG. 8B, and FIG. 8C graphically depict the amount of three tryptic peptides of tau, LQTA (FIG. 8A), HVPG (FIG. 8B), and IGSL (FIG. 8C), respectively, in CSF samples processed by the PostIP-CX method described in Examples 1 and 2. Longitudinal samples from individual patients are shown with amyloid status indicated by symbols (CDR0=black circle, CDR0.5=blue triangle, CDR1=red square, CDR2=purple reverse-triangle). Paired t-test was used for 1^{st} and 2^{nd} visit results from individual participants. The amyloid positive group showed significant changes in direction for each patient. Also notable are the changes observed for participant A (denoted by the bolded red line), whose tau-PET signal was high (>2 SUVR) and whose CDR score changed from CDR1 to CDR2. For this patient, LQTA was increased (FIG. 8A), HVPG was decreased (FIG. 8B), and IGSL was decreased (FIG. 8C) due to the progression of tau pathology.

FIG. 9A, FIG. 9B, and FIG. 9C graphically depict the amount of three tryptic peptides of tau in CSF samples processed by the PostIP-CX method described in Examples 1 and 2 vs. CDR-SB score for the subject from whom the sample was obtained. The three tryptic peptides of tau are LQTA, HVPG, and IGSL, respectively. Amyloid positive subjects are blue circles; amyloid negative subjects are red squares. As shown by the accompanying statistical analyses, only LQTA is significantly correlated with CDR-SB.

FIG. 10A, FIG. 10B, and FIG. 10C graphically depicts the amount of three tryptic peptides of tau in CSF samples processed by the PostIP-CX method described in Examples 1 and 2 vs. mini-mental state examination (MMSE) score for the subject from whom the sample was obtained. The three tryptic peptides of tau are LQTA, HVPG, and IGSL, respectively. Amyloid positive subjects are blue circles; amyloid negative subjects are red squares. As shown by the accompanying statistical analyses, only LQTA is significantly correlated with MMSE.

FIG. 11A, FIG. 11B, and FIG. 11C graphically depict the amount of three tryptic peptides of tau in CSF samples processed by the PostIP-CX method described in Examples 1 and 2 vs. Tau-PET score for the subject from whom the sample was obtained. The three tryptic peptides of tau are LQTA, HVPG, and IGSL, respectively. Amyloid positive subjects are blue circles; amyloid negative subjects are red squares. As shown by the accompanying statistical analyses, only LQTA is significantly correlated with Tau-PET. The other tryptic peptides of MTBR tau did not.

FIG. 12A and FIG. 12B graphically depict the amount of the tryptic peptide of tau, LQTA, in CSF samples processed by the PostIP-CX method described in Examples 1 and 2 vs. CDR-SB (FIG. 12A) and MMSE (FIG. 12B). The data shown LQTA showed a significant correlation with cognitive function, as evaluated by two different measures of cognitive impairment.

FIG. 13A graphically depicts the amount of the tryptic peptide of tau, LQTA, in samples processed by the IP method (x-axis) and the PostIP-CX method (y-axis). Amyloid positive subjects are identified with red symbols; amyloid negative subjects are identified with blue symbols. As shown by the accompanying statistical analyses, only “MTBR-related LQTA” (measured in samples processed by the PostIP-CX method) showed more increase in amyloid positive group than in the amyloid negative group.

FIG. 13B and FIG. 13C graphically depict the amount of the tryptic peptide of tau, LQTA, in CSF samples processed by the PostIP-CX method or IP method, respectively, as described in Examples 1 and 2. The CSF samples are grouped by CDR score and amyloid status. The data show the LQTA-specific characteristic (i.e., linear increase after symptomatic stage) was observed for only “MTBR-related LTQA”. Data are represented as the individual results (plots) and the mean (bar). Significance in statistical test: ****p<0.001, ***p<0.001, **p<0.01, *p<0.05. NS=not significant. Statistical differences were assessed with one-way ANOVA with multiple comparisons correction using Benjamini-Hochberg false discovery rate (FDR) method with FDR set at 5%.

FIG. 14 is a graph showing a receiver operator curve (ROC) comparing the sensitivity and specificity of the tryptic peptide of tau, LQTA, measured by mass spectrometry following the IP method (blue (bottom) line) or the PostIP-CX method (red (top) line), for determining amyloid status. The curves show that PostIP-LQTA (MTBR-related LQTA) clearly discriminates amyloid status better than IP-LQTA.

FIG. 15A and FIG. 15B show a ratio of IGSL to HVPG boosts the discrimination power. FIG. 15A graphically depicts the amount of the tryptic peptides of tau, IGSL and LQTA expressed as a ratio (IGSL/LQTA), in CSF samples processed by the PostIP-CX method, described in Examples 1 and 2. The CSF samples are grouped by CDR score and amyloid status. FIG. 15B graphically shows the relationship between the IGSL/LQTA ratio and pT205%. pT205% was measured as previously described (Barthélemy, N. R., Li, Y, Joseph-Mathurin, N. et al. Nat Med 26, 398-407 (2020)). IGSL/LQTA shows a very tight correlation with pT205, which is modulated at close to AD onset.

FIG. 15C and FIG. 15D show a ratio of IGSL to HVPG boosts the discrimination power. FIG. 15C graphically depicts the amount of the tryptic peptides of tau, IGSL and HVPG expressed as a ratio (IGSL/HVPG), in CSF samples processed by the PostIP-CX method, described in Examples 1 and 2. The CSF samples are grouped by CDR score and amyloid status. FIG. 15D graphically shows the relationship between the IGSL/LQTA ratio and pT217%. IGSL/HVPG shows a very tight correlation with pT217, which recapitulates amyloid status.

FIG. 16 is an illustration showing tau pathology evolves through distinct phases in Alzheimer's disease. Measuring four different soluble tau species and insoluble tau in a group of participants with deterministic Alzheimer disease mutations we show over the course of about 40 years (x-axis) tau related changes unfold (y-axis) and differ based on the stage of disease and other measurable biomarkers. Starting with the development of fibrillar amyloid pathology phosphorylation at position 217 (purple) and 181 (blue) begins to increase. With the increase in neuronal dysfunction (based metabolic changes) phosphorylation at position 205 (green) begins to increase along with soluble tau (orange). Lastly, with the onset of neurodegeneration (based on brain atrophy and cognitive decline) tau PET tangles (red) begin to develop while phosphorylation of 217 and 181 begins to decrease. Together, this highlights the dynamic and diverging patterns of soluble and aggregated tau over the course of the disease and close relationship with amyloid pathology.

FIG. 17 is an illustration of a theoretical model showing how accessibility of different regions of MTBR tau to cleavage may vary during Alzheimer's disease (AD) progression, and why MTBR tau species comprising the amino acid sequence of SEQ ID NO: 3 (LQTAPVPMPDLK) is a good surrogate for tau-pathology all across AD stages. In preclinical AD stages, brain tau aggregates are immature, allowing proteases greater access. MTBR tau species comprising MTBR tau-243, MTBR tau-299, and MTBR tau-354 are secreted into CSF. However, as tau aggregates mature with disease progression and form an increasingly rigid core, protease access to MTBR tau-354 and then MTBR tau-299 decreases, and MTBR-species comprising MTBR tau-354 and then MTBR tau-299 stabilize in the CSF. MTBR tau-243, however, remains exposed, enabling protease digestion and release into CSF at all disease stages. The imbalance for these three species in CSF is observed as a reflection of brain tau aggregate formation. Note: in this illustration, differences in size between MTBR tau species is not depicted.

FIG. 18A is a schematic of tryptic peptides from tau (grey bars) that were quantified in Example 3, and further discussed in FIG. 18B and FIG. 18C.

FIG. 18B and FIG. 18C are graphs showing brain MTBR tau species comprising MTBR tau-243, 299 and 354 are enriched in aggregated Alzheimer's disease brain insoluble extracts compared to control brain extracts, confirming that MTBR tau is specifically deposited in Alzheimer's disease brain. The graphs show the enrichment profile of tau peptides from (FIG. 18B) control and Alzheimer's disease brains (n=2 with six-eight brain regions samples/group in discovery cohort) and (FIG. 18C) from control (amyloid-negative, n=8), very mild to moderate Alzheimer's disease (AD) (amyloid-positive, CDR=0.5−2, n=5), and severe AD brains (amyloid-positive, CDR=3, n=7) (total n=20 in validation cohort). The relative abundance of tau peptides was quantified relative to the mid-domain (residue 181-190) peptide for internal normalization. The species containing the upstream region of microtubule binding region (MTBR) domain (residue 243-254, MTBR tau-243) and repeat region 2 (R2) to R3 and R4 (residues 299-317, MTBR tau-299 and 354-369, MTBR tau-354, respectively) were highly enriched in the insoluble fraction from Alzheimer's disease brains compared to controls and were specifically enriched by clinical stage of disease progression as measured by the CDR. MTBR tau-299 and MTBR tau-354 are located inside the filament core, whereas MTBR tau-243 is located outside the core of Alzheimer's disease aggregates (Fitzpatrick et al., 2017). Of note, residue 195-209 was decreased in Alzheimer's disease brains, potentially due to a high degree of phosphorylation. Data are represented as box-and-whisker plots with Tukey method describing median, interquartile interval, minimum, maximum, and individual points for outliers. Significance in statistical test: ****p<0.001, ***p<0.001, **p<0.01, *p<0.05.

FIG. 19A is a schematic of tryptic peptides from tau (grey bars) that were quantified in Example 3, and further discussed in FIG. 19B and FIG. 19C, as well as the general binding site of the antibodies HJ8.5 and Tau1.

FIG. 19B is a graph showing the tau profile in control human CSF. Tau peptides in control human CSF from a cross-sectional cohort of amyloid-negative and CDR=0 patients (n=30) were quantified by Tau1/HJ8.5 immunoprecipitation focusing on N-terminal to mid-domain tau. To quantify the species containing the microtubule binding region (MTBR) and C-terminal region, post-immunoprecipitated CSF samples were chemically extracted and analyzed sequentially. Using the Tau1/HJ8.5 immunoprecipitation method (blue circle), peptide recovery dramatically decreased after reside 222; therefore, only N-terminal to mid-domain tau (residues 6-23 to 243-254) peptides were quantified by this method (Sato et al., 2018). In contrast, the chemical extraction method of post-immunoprecipitated CSF (red square) enabled quantification of whole regions of tau including the MTBR to C-terminal regions at concentrations between 0.4-7 ng/mL. Data are represented as means.

FIG. 20A, FIG. 20B, and FIG. 20C are graphs showing the amount of mid-domain-independent MTBR tau-243 (FIG. 20A), mid-domain-independent MTBR tau-299 (FIG. 20B), and mid-domain-independent MTBR tau-354 (FIG. 20C) in PostIP-CX CSF from the cross-sectional cohort. Mid-domain-independent MTBR tau-243, mid-domain-independent MTBR tau-299 and mid-domain-independent MTBR tau-354 show different profiles to amyloid plaques and clinical dementia stage. Amyloid-negative CDR=0 (control, n=30), amyloid-positive CDR=0 (preclinical AD, n=18), amyloid-positive CDR=0.5 (very mild AD, n=28), amyloid-positive CDR≥1 (mild-moderate AD, n=12), and amyloid-negative CDR≥0.5 (non-AD cognitive impairment, n=12). Mid-domain-independent MTBR tau-243 showed a continuous increase with Alzheimer's disease progression through all clinical stages. Mid-domain-independent MTBR tau-299 and mid-domain-independent MTBR tau-354 concentrations similarly increased until the very mild Alzheimer's disease stage (amyloid-positive and CDR=0.5), but then either saturated (MTBR tau-299) or decreased (MTBR tau-354) at CDR≥1. The p-values in red or blue fonts indicate a significant increase or decrease, respectively. Data are represented as the individual results (plots) and the mean (bar). Significance in statistical test: ****p<0.001, ***p<0.001, **p<0.01, *p<0.05. NS=not significant.

FIG. 21A, FIG. 21B, and FIG. 21C are graphs showing longitudinal rates of changes (ng/mL/year) in (FIG. 21A) mid-domain-independent MTBR tau-243, (FIG. 21B) mid-domain-independent MTBR tau-299, and (FIG. 21C) mid-domain-independent MTBR tau-354 in CSF in amyloid-negative (−) or positive (+) patients are shown (total n=28 from longitudinal cohort). Amyloid-positive groups are further divided to various CDR changes including CDR=0−0 (n=7), CDR=0−0.5 (n=2), CDR=0−1 (n=1), CDR=0.5−0.5 (n=2), CDR=0.5−1 (n=1), and CDR=1−2 (n=1, participant A). While the participants in the amyloid-negative group did not show significant longitudinal changes (as mean-values are close to zero), most participants in the amyloid-positive group showed increases in MTBR tau concentrations longitudinally. Notably, participant A with the greatest cognitive change after Alzheimer's disease clinical onset (CDR=1 to 2) demonstrated only CSF MTBR tau-243 increased with mild AD (CDR=1) to moderate AD (CDR=2) progression, while MTBR tau-299 and 354 decreased. These data show CSF mid-domain-independent MTBR tau species longitudinally increase with advancing Alzheimer's disease clinical stages.

FIG. 22A, FIG. 22B, and FIG. 22C are graphs showing (x-axis) tau PET (AV-1451) SUVR and (y-axis) mid-domain-independent (FIG. 22A) MTBR tau-243, (FIG. 22B) MTBR tau-299, and (FIG. 22C) MTBR tau-354 concentrations in PostIP-CX CSF (control n=15 and Alzheimer's disease (AD) n=20 from tau PET cohort). Open circle: control, filled squares: AD. Mid-domain-independent MTBR tau-243 showed the most significant correlation with tau PET SUVR (r=0.7588, p<0.0001). These data show CSF mid-domain-independent MTBR tau species comprising SEQ ID NO: 3 (LQTAPVPMPDLK) is highly correlated with tau PET SUVR measure of tau tangles, while other mid-domain-independent MTBR tau regions have lower correlations with tau tangles.

FIG. 23A and FIG. 23B graphically depict that brain MTBR tau-243, MTBR tau-299, and MTBR tau-354 are not enriched in Alzheimer's disease brain soluble extracts compared to control brain extracts. FIG. 23A shows the enrichment profile of tau peptides from control and Alzheimer's disease brains (n=2 with eight-ten brain regions samples/group in discovery cohort) and FIG. 23B shows the enrichment profile of tau peptides from control (amyloid-negative, n=8), very mild to moderate Alzheimer's disease (AD) (amyloid-positive, CDR=0.5−2, n=5), and severe AD brains (amyloid-positive, CDR=3, n=7) in the validation cohort (total n=20). Relative peptide abundance of tau peptides was quantified relative to mid-domain (residue 181-190) peptide for internal normalization. No changes were observed for tau species containing microtubule binding region (MTBR) domain in soluble tau species, in contrast to the insoluble MTBR tau species which were increased in Alzheimer's disease brains (FIG. 18). Data are represented as box-and-whisker plots with Tukey method describing median, interquartile interval, minimum, maximum, and individual points for outlier. Significance in statistical test: **p<0.01, *p<0.05.

FIG. 24 is a graph showing that MTBR tau-354 correlates with tau368 in brain insoluble extracts, suggesting each species is not differentiated by progression of tau pathology. Mass spectrometry analysis of MTBR tau-354 and tau368 species in brain insoluble extracts from control and Alzheimer's disease patients was conducted using discovery cohort samples (total 23 brain samples, six brain regions from Alzheimer's disease #1 participant, eight brain regions from Alzheimer's disease #2 participant, five brain regions from Control #1 participant, four brain regions from Control #2 participant). MTBR tau-354 (residue 354-369) and its truncated form, tau368 (residue 354-368), exhibited a tight correlation in brain insoluble extracts (Spearman r=0.9783).

FIG. 25A, FIG. 25B, FIG. 25C, FIG. 25D, FIG. 25E and FIG. 25F are graphs showing the quantification of tryptic peptides of MTBR tau in a human CSF sample after processing by the PostIP-CX method followed by mass spectrometry (MS) analysis. Extracted MS chromatograms of mid-domain-independent MTBR tau-243 (FIG. 25A, FIG. 25D), mid-domain-independent MTBR tau-299 (FIG. 25B, FIG. 25E), and mid-domain-independent MTBR tau-354 (FIG. 25C, FIG. 25F) are shown. The human CSF was obtained from an amyloid positive and CDR=0.5 very mild Alzheimer's disease participant from the cross-sectional cohort. FIG. 25A, FIG. 25B, and FIG. 25C show the peaks from endogenous tryptic peptides, while FIG. 25D, FIG. 25E, and FIG. 25F show the peaks from an internal standard (¹⁵N-labeled tau). X-axis and Y-axis indicate the retention time and MS intensity of each peak, respectively.

FIG. 26A, FIG. 26B, FIG. 26C, FIG. 26D, FIG. 26E, FIG. 26F, FIG. 26G, FIG. 26H, FIG. 26I, FIG. 26J, and FIG. 26K are graphs showing the concentration of tryptic peptides of N-terminal tau and mid-domain tau in human CSF after sample processing by the IP method followed by MS analysis. N-terminal and mid-domain CSF tau species distinguish very early dementia from normal, but do not correlate with dementia stage. Tau species, residues (FIG. 26A) 6-23, (FIG. 26B) 25-44, (FIG. 26C) 45-67, (FIG. 26D) 68-87, (FIG. 26E) 88-126, (FIG. 26F) 151-155, (FIG. 26G) 181-190, (FIG. 26H) 195-209, (FIG. 26I) 212-221, (FIG. 26J) 226-230, and (FIG. 26K) 243-254 concentrations in CSF from groups within the cross-sectional cohort (residue numbering based on tau-441). Amyloid-negative CDR=0 (control, n=29), amyloid-positive CDR=0 (preclinical AD, n=18), amyloid-positive CDR=0.5 (very mild AD, n=27), amyloid-positive CDR≥1 (mild-moderate AD, n=12), and amyloid-negative CDR≥0.5 (non-AD clinical impairment, n=12). These tau species were isolated using the Tau1/HJ8.5 immunoprecipitation method (IP method). The p-values in red font indicate a significance. Data are represented as the individual results (plots) and the mean (bar). Significance in statistical test: ****p<0.001, ***p<0.001, **p<0.01, *p<0.05. NS=not significant.

FIG. 27A, FIG. 27B, FIG. 27C, FIG. 27D, FIG. 27E, FIG. 27F, FIG. 27G, and FIG. 27H are graphs showing the concentration of tryptic peptides of MTBR tau in human CSF after sample processing by the PostIP-CX method followed by MS analysis. Unique Alzheimer's disease amyloid and clinical staging patterns are specific to mid-domain-independent MTBR tau species in CSF. Only mid-domain-independent MTBR tau-243 distinguishes more advanced clinical stages. Tau species, residues (FIG. 27A) 243-254 (MTBR tau-243), (FIG. 27B) 260-267, (FIG. 27C) 275-280, (FIG. 27D) 282-290, (FIG. 27E) 299-317 (MTBR tau-299), (FIG. 27F) 354-369 (MTBR tau-354), (FIG. 27G) 386-395, and (FIG. 27H) 396-406 concentrations in CSF obtained using chemical extraction from post-immunoprecipitated samples from participants within the cross-sectional cohort (residue numbering based on tau-441). Amyloid-negative CDR=0 (control, n=30), amyloid-positive CDR=0 (preclinical AD, n=18), amyloid-positive CDR=0.5 (very mild AD, n=28), amyloid-positive CDR≥1 (mild-moderate AD, n=12), and amyloid-negative CDR≥0.5 (non-AD clinical impairment, n=12). The p-values in red or blue fonts indicate a significant increase or decrease, respectively. Data are represented as the individual results (plots) and the mean (bar). Significance in statistical test: ****p<0.001, ***p<0.001, **p<0.01, *p<0.05. NS=not significant.

FIG. 28A, FIG. 28B, FIG. 28C, FIG. 28D, FIG. 28E, FIG. 28F, FIG. 28G, FIG. 28H, FIG. 28I, and FIG. 28J are graphs showing the concentration of tryptic peptides of N-terminal tau and mid-domain tau in human CSF after sample processing by the PostIP-CX method followed by MS analysis. N-terminal and mid-domain CSF tau species do not correlate with dementia stage regardless of the purification method (see also FIG. 26). Tau species, residues (FIG. 28A) 6-23, (FIG. 28B) 25-44, (FIG. 28C) 45-67, (FIG. 28D) 68-87, (FIG. 28E) 88-126, (FIG. 28F) 151-155, (FIG. 28G) 181-190, (FIG. 28H) 195-209, (FIG. 28I) 212-221, and (FIG. 28J) 226-230, concentrations in CSF from groups within the cross-sectional cohort. Amyloid-negative CDR=0 (control, n=29), amyloid-positive CDR=0 (preclinical AD, n=18), amyloid-positive CDR=0.5 (very mild AD, n=27), amyloid-positive CDR≥1 (mild-moderate AD, n=12), and amyloid-negative CDR≥0.5 (non-AD clinical impairment, n=12). These tau species were isolated using the Tau1/HJ8.5 immunoprecipitation method followed by chemical extraction of the post-immunoprecipitated CSF (PostIP-CX). The sums of concentrations from the immunoprecipitation method and chemical extraction method for post-immunoprecipitated CSF are shown for N-terminal to mid domain tau species (as total concentrations). The p-values in red font indicate statistical significance. Data are represented as the individual results (plots) and the mean (bar). Significance in statistical test: ****p<0.001, ***p<0.001, **p<0.01, *p<0.05. NS=not significant.

FIG. 28K is a graph showing the total concentration of tau species containing residues 243-254 (sum concentration from the IP method and the PostIP-CX method).

FIG. 29 graphically shows that mid-domain-independent MTBR tau-354 correlates with mid-domain-independent tau368 in CSF. CSF was processed by the PostIP-CX method as generally described in Example 3. Mid-domain-independent MTBR tau-354 (residue 354-369) and its truncated form, tau368 (residue 354-368), display a tight correlation (Spearman r=0.8382). Results were obtained from cross-sectional cohort samples (n=100 including all clinical stages).

FIG. 30A, FIG. 30B, and FIG. 30C are graphs showing that CSF mid-domain-independent MTBR tau-243 highly correlates with Clinical dementia rating-sum of boxes (CDR-SB), while mid-domain-independent MTBR tau-299 and mid-domain-independent MTBR tau-354 do not. CDR-SB correlations with (FIG. 30A) mid-domain-independent MTBR tau-243, (FIG. 30B) mid-domain-independent MTBR tau-299, and (FIG. 30C) mid-domain-independent MTBR tau-354 concentrations in CSF. Mid-domain-independent MTBR tau-243 in CSF from amyloid positive groups showed a high correlation with CDR-SB (r=0.5562, p<0.0001). Results were obtained from cross-sectional cohort samples (amyloid-negative n=42 and amyloid-positive n=58).

FIG. 31A, FIG. 31B, and FIG. 31C are graphs showing that CSF mid-domain-independent MTBR tau-243 highly correlates with mini-mental state exam (MMSE) more than mid-domain-independent MTBR tau-299 and mid-domain-independent MTBR tau-354. MMSE) correlations with (FIG. 31A) mid-domain-independent MTBR tau-243, (FIG. 31B) mid-domain-independent MTBR tau-299, and (FIG. 31C) mid-domain-independent MTBR tau-354 concentrations in CSF. Mid-domain-independent MTBR tau-243 in CSF from amyloid positive groups showed a high correlation with MMSE (r=0.5433, p<0.0001). Results were obtained from cross-sectional cohort samples (amyloid-negative n=42 and amyloid-positive n=58).

FIG. 32A, FIG. 32B, and FIG. 32C are graphs showing CSF mid-domain-independent MTBR tau-243, mid-domain-independent MTBR tau-299, mid-domain-independent MTBR tau-354 longitudinally increase with advancing Alzheimer's disease clinical stages. Longitudinal changes in mid-domain-independent (FIG. 32A) MTBR tau-243, (FIG. 32B) MTBR tau-299, and (FIG. 32C) MTBR tau-354 tau concentrations in CSF in amyloid negative (−) or positive (+) patients are shown. Black circle: CDR=0, Blue triangle: CDR=0.5, Red square: CDR=1, Purple reverse-triangle: CDR=2. The participants with stable (or decreasing) CDR trajectory are represented by a dotted line. The participants with increasing CDR between the 1st and 2nd visits are represented by a solid line. The bolded red line in the amyloid-positive group shows the longitudinal trajectory of a specific participant (participant A) with the greatest cognitive change after Alzheimer's disease onset (CDR=1 to 2), indicating CSF MTBR tau-243 increased even in mild AD (CDR=1) to moderate AD (CDR=2). Statistical significance was evaluated by paired t-test for 1^{st} and 2^{nd} visits (amyloid-negative n=14 and amyloid-positive n=14). **p<0.01. NS=not significant.

FIG. 33 is a schematic illustrating a method of the present disclosure.

FIG. 34A, FIG. 34B, and FIG. 34C are graphs depicting the amount of the tryptic peptides LQTA (FIG. 34A), HVPG (FIG. 34B) and IGSL (FIG. 34C) measured in CSF samples processed by the PostIP-CX method (top) vs. the PostIP-CX method (bottom).

FIG. 35A graphically shows the amount of the tryptic peptides LQTA (left), IGST (middle) and VQII (right) measured in samples processed by the PostIP-IP method (y-axis) vs. the PostIP-CX method (x-axis). The illustration above the graphs shows the relative location of each tryptic peptide in tau-441. Both axes show absolute concentrations (ng/mL).

FIG. 35B graphically shows the amount of the tryptic peptides LDLS (left), HVPG (middle) and IGSL (right) measured in samples processed by the PostIP-IP method (y-axis) vs. the PostIP-CX method (x-axis). The illustration above the graphs shows the relative location of each tryptic peptide in tau-441. Both axes show absolute concentrations (ng/mL).

FIG. 36A graphically shows the amount of the tryptic peptides LQTA (left), IGST (middle) and VQII (right) measured in samples processed by the PostIP-IP method (y-axis) vs. the PostIP-CX method (x-axis). The illustration above the graphs shows the relative location of each tryptic peptide in tau-441. Both axes show absolute concentrations (ng/mL). Samples obtained from control subjects are blue circles; Samples obtained from amyloid positive subjects without cognitive impairment (CDR<0.5) are red squares; and samples obtained from amyloid positive subjects with cognitive impairment (CDR>0.5) are black triangles.

FIG. 36B graphically shows the amount of the tryptic peptides LDLS (left), HVPG (middle) and IGSL (right) measured in samples processed by the PostIP-IP method (y-axis) vs. the PostIP-CX method (x-axis). The illustration above the graphs shows the relative location of each tryptic peptide in tau-441. Both axes show absolute concentrations (ng/mL). Samples obtained from control subjects are blue circles; Samples obtained from amyloid positive subjects without cognitive impairment (CDR<0.5) are red squares; and samples obtained from amyloid positive subjects with cognitive impairment (CDR>0.5) are black triangles.

FIG. 37A is an illustration of the various full-length tau isoforms. The relative locations of several tryptic peptides of tau are indicated (e.g., LQTA, IGST, VQII, LDLS, HVPG, IGSL, VQIV). Each “Y” represents an antibody that specifically binds within the N-terminus (left) and mid-domain (middle) and MTBR (right) regions. The main cleavage site of tau, which is at amino acid 224 of tau-441, is depicted as a dashed line.

FIG. 37B graphically shows the amount of the tryptic peptides VQIV and LDLS, expressed as a ratio, in samples obtained from control subjects (left, blue circles) and subjects with non-AD tauopathies (right, green circles), as determined by LC-MS following sample processing by the IP method described in Example 1. ns not significant; Tukey's multiple comparisons tests.

FIG. 37C graphically shows the amount of the tryptic peptides VQIV and LDLS, expressed as a ratio, in samples obtained from control subjects (left, blue circles) and subjects with non-AD tauopathies (right, green circles), as determined by LC-MS following sample processing by the PostIP-IP method described in Example 4.

FIG. 38 graphically shows the amount of the tryptic peptides VQIV (x-axis) and LDLS (y-axis) in samples obtained from control subjects (blue circles) and subjects with non-AD tauopathies (green triangles) as determined by LC-MS following sample processing by the PostIP-IP method described in Example 4. ****p<0.0001; Tukey's multiple comparison test.

FIG. 39 graphically shows the amount of the tryptic peptides VQIV and LDLS, expressed as a ratio, in samples obtained from control subjects (left, blue circles), subjects with AD (middle, red circles), and subjects with non-AD tauopathies (right, green circles), as determined by LC-MS following sample processing by the PostIP-IP method described in Example 4. ns not significant; ****p<0.0001; Tukey's multiple comparison test.

FIG. 40A, FIG. 40B, FIG. 40C, FIG. 40D, FIG. 40E, and FIG. 40F graphically show comparisons of the amounts of the tryptic peptides of tau in samples obtained from control subjects (blue circles), subjects with AD (red squares), and subjects with non-AD tauopathies (green triangles), as determined by LC-MS following sample processing by the PostIP-IP method described in Example 4. The tryptic peptides of tau are IGST (x-axis) and VQII (y-axis) in FIG. 40A, LDLS (x-axis) and VQII (y-axis) in FIG. 40B, IGSL (x-axis) and HVPG (y-axis) in FIG. 40C, IGST (x-axis) and HPVG (y-axis) in FIG. 40E, VQII (x-axis) and HPVG (y-axis) in FIG. 40E, and IGST (x-axis) and HPVG (y-axis) in FIG. 40F. Both axes show absolute concentrations (ng/mL).

FIG. 41 graphically shows comparisons of the amounts of the tryptic peptides of tau IGST vs. HVPG (top left), VQII vs. HVPG (top right), and LDLS vs. HVPG (bottom) in samples obtained from subjects with non-AD tauopathies, as determined by LC-MS following sample processing by the PostIP-IP method described in Example 4. The key at the right identifies the non-AD tauopathy diagnosis for each subject. Both axes show absolute concentrations (ng/mL).

FIG. 42A, FIG. 42B, and FIG. 42C graphically show comparisons of the amounts of the tryptic peptides of tau IGST vs. IGSL (FIG. 42A), VQII vs. IGSL (FIG. 42B), and LDLS vs. IGSL (FIG. 42C) in samples obtained from control subjects (blue circles), subjects with AD (red squares), and subjects with non-AD tauopathies (green triangles), as determined by LC-MS following sample processing by the PostIP-IP method described in Example 4. Both axes show absolute concentrations (ng/mL).

FIG. 43A is an illustration of an MTBR region of tau. The relative positions of the tryptic peptides IGST, VQII, LDLS, HVPG, and IGSL is shown, as is the relative position of the epitope that antibody 77G7 specifically binds.

FIG. 43B, FIG. 43C, FIG. 43D, and FIG. 43E graphically show the ratio of the tryptic peptides of tau IGSL/IGST (FIG. 43B), IGSL/VQII (FIG. 43C), IGSL/LDLS (FIG. 43D), and IGSL/HVPG (FIG. 43E) in samples obtained from non-AD subjects (left bar), subjects with AD (right bar), as determined by LC-MS following sample processing by the PostIP-IP method described in Example 4. For the non-AD subjects, blue indicates subjects with PSP, green indicates subjects with FTD, red indicates subjects with CBD, and purple indicates subjects with PSP-CBD continuous. Statistical significance was determined by unpaired t-test with Welch's correction.

FIG. 44A, FIG. 44B, FIG. 44C, FIG. 44D, and FIG. 44E graphically show comparisons of the amounts of the tryptic peptides of tau IGSL vs. IGST (FIG. 44A), IGSL vs. VQII (FIG. 44B), IGSL vs. LDLS (FIG. 44C), VQII vs. IGST (FIG. 44D), VQII vs. LDLS (FIG. 44E), IGSL vs. HVPG (FIG. 43E) in samples obtained from non-AD subjects (blue), and subjects with AD (red), as determined by LC-MS following sample processing by the PostIP-IP method described in Example 4. In FIG. 44A, FIG. 44B, and FIG. 44C, non-AD subjects showed the scattered plots, whereas in FIG. 44D, FIG. 44E, and FIG. 44F AD and non-AD showed identical correlations.

FIG. 45 shows the relationship among various tryptic peptides of tau as measured in CSF from subjects with AD and non-AD tauopathies. The data highlighted in the box suggest a differentiation point for discriminating AD and non-AD tauopathies.

FIG. 46 is an illustration depicting a hypothesis for how CSF tau discriminates non-AD tauopathies. As depicted, in CSF, non-AD tauopathies contain (1) less R1-R2 and (2) more R3-R4 than AD, as a reflection of brain tau deposition.

FIG. 47 graphically shows the amount of various tryptic peptide of brain insoluble tau.

FIG. 48A, FIG. 48B, FIG. 48C, and FIG. 48D graphically show the ratio of the tryptic peptides of tau IGSL/IGST (FIG. 48A), IGSL/VQII (FIG. 48B), IGSL/LDLS (FIG. 48C), and IGSL/HVPG (FIG. 48D) in samples obtained from control subjects (left bar), subjects with AD (middle bar), and non-AD subjects (right bar), as determined by LC-MS following sample processing by the PostIP-IP method described in Example 4. For the non-AD subjects, black triangles indicate subjects with genetically confirmed P301 L FTLD (4R-tauopathy) and black squares indicate subjects with genetically confirmed R406W FTLD (3R/4R mix).

FIG. 49A, FIG. 49B, FIG. 49C, FIG. 49D, FIG. 49E, and FIG. 49F graphically show comparisons of the amounts of the tryptic peptides of tau IGSL vs. IGST (FIG. 49A), IGSL vs. VQII (FIG. 49B), IGSL vs. LDLS (FIG. 49C), VQII vs. IGST (FIG. 49D), VQII vs. LDLS (FIG. 49E), IGSL vs. HVPG (FIG. 43F) in CSF samples obtained from control subjects (blue), subjects with AD (red), and non-AD subjects (green) as determined by LC-MS following sample processing by the PostIP-IP method described in Example 4. In FIG. 49A, FIG. 49B, and FIG. 49C, non-AD subjects showed the scattered plots, whereas in FIG. 49D, FIG. 49E, and FIG. 49F control, AD and non-AD showed identical correlations.

### DETAILED DESCRIPTION

MTBR tau exists as a plurality of peptides in blood and CSF. Detection and quantification of MTBR tau in these biological samples has been hampered due to the very low abundance of these polypeptides. The methods disclosed herein employ unique combinations of processing steps that transform a biological sample into a sample suitable for quantifying MTBR tau, as well as other tau species. For instance, in some methods of the present disclosure, the processing steps deplete certain proteins while enriching for a plurality of tau proteins. In other methods of the present disclosure, the processing steps deplete certain proteins while enriching for a plurality of MTBR tau proteins. Certain methods disclosed herein are particularly suited for quantifying mid-domain-independent MTBR tau species. Also described herein are uses of mid-domain-independent MTBR tau species to measure clinical signs and symptoms of tauopathies, diagnose tauopathies, and direct treatment of tauopathies. These and other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

An antibody, as used herein, refers to a complete antibody as understood in the art, i.e., consisting of two heavy chains and two light chains, and also to any antibody-like molecule that has an antigen binding region, including, but not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11C]PIB-PET imaging with mean cortical binding potential (MCBP) score >0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patent Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer's disease), based on qualitative or quantitative test results.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due to alternative splicing of mRNA encoding the protein, post-translational modification of the protein, proteolytic processing of the protein, genetic variations and somatic recombination. The terms “isoform” and “variant” are used interchangeably.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, non-human primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (0N). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, 1N3R, 1N4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

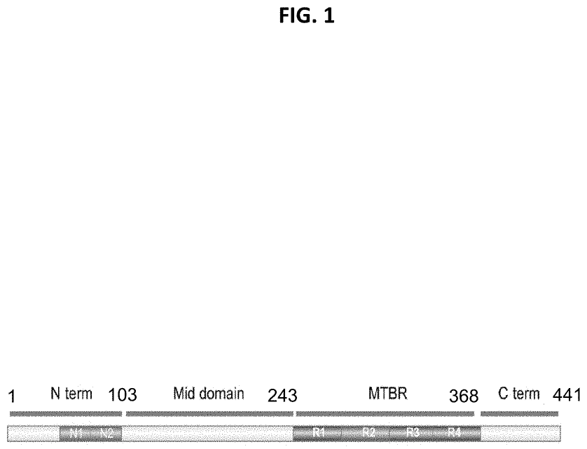

The term “tau-441,” as used herein, refers to the longest human tau isoform (2N4R), which is 441 amino acids in length. The amino acid sequence of tau-441 is provided as SEQ ID NO: 1. The N-terminus (N term), mid-domain, MTBR, and C-terminus (C term) are identified in FIG. 1 for this isoform. These regions will vary in a predictable way for other tau isoforms (e.g., 2N3R, 1NR4, 1N3R, 0N4R, and 0N3R). Accordingly, when amino acid positions are identified relative to tau-441, a skilled artisan will be able to determine the corresponding amino acid position for the other isoforms.

The term “N-terminal tau,” as used herein, refers to a tau protein, or a plurality of tau proteins, that comprise(s) two or more amino acids of the N-terminus of tau (e.g., amino acids 1-103 of tau-441, etc.).

The term “mid-domain tau,” as used herein, refers to a tau protein, or a plurality of tau proteins, that comprise(s) two or more amino acids of the mid-domain of tau (e.g., amino acids 104-243 of tau-441, etc.).

The term “MTBR tau,” as used herein, refers to a tau protein, or a plurality of tau proteins, that comprise(s) two or more amino acids of the microtubule binding region (MTBR) of tau (e.g., amino acids 244-368 of tau-441, etc.).

The term “C-terminal tau,” as used herein, refers to a tau protein, or a plurality of tau proteins, that comprise(s) two or more amino acids of the C-terminus of tau (e.g., amino acids 369-441 of tau-441, etc.).

A “proteolytic peptide of tau” refers to a peptide fragment of a tau protein produced by in vitro proteolytic cleavage. A “tryptic peptide of tau” refers to a peptide fragment of a tau protein produced by in vitro cleavage with trypsin. Tryptic peptides of tau may be referred to herein by their first four amino acids. For instance, “LQTA” refers to the tryptic peptide LQTAPVPMPDLK (SEQ ID NO: 3). Non-limiting examples of other tryptic peptides identified by their first four amino acids include IGST (SEQ ID NO: 2), VQII (SEQ ID NO: 4), LDLS (SEQ ID NO: 5), HVPG (SEQ ID NO: 6), IGSL (SEQ ID NO: 7), VQIV (SEQ ID NO: 9), and TPPS (SEQ ID NO: 10).

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration (CBD), argyrophilic grain disease (AGD), Frontotemporal lobar degeneration (FTLD), Alzheimer's disease (AD), and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick's disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer's disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles. Methods for detecting and quantifying tau aggregates in the brain are known in the art (e.g., tau PET using tau-specific ligands such as [18F]THK5317, [18F]THK5351, [18F]AV1451, [11C]PBB3, [18F]MK-6240, [18F]RO-948, [18F]PI-2620, [18F]GTP1, [18F]PM-PBB3, and [18F]JNJ64349311, [18F]JNJ-067), etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthioninium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1 D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

### II. Methods for Measuring Tau

The present disclosure provides methods for measuring tau in a biological sample by mass spectrometry. Generally speaking, methods of the present disclosure for measuring tau in a biological sample comprise providing a biological sample, processing the biological sample by depleting one or more protein and then purifying tau, cleaving the purified tau with a protease and then optionally desalting the resultant cleavage product by solid phase extraction to obtain a sample comprising proteolytic peptides of tau, and performing liquid chromatography-mass spectrometry with the sample comprising proteolytic peptides of tau to detect and measure the concentration (relative or absolute) of at least one proteolytic peptide of tau. Thus, in practice, the disclosed methods use at least one proteolytic peptide of tau to detect and measure the amount of tau present in the biological sample.

In one example, a method of the present disclosure comprises (a) providing a biological sample selected from a blood sample or a CSF sample; (b) removing proteins from the biological sample by protein precipitation and separating the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography-mass spectrometry with the sample comprising proteolytic peptides of tau to detect and measure the concentration of at least one proteolytic peptide of tau.

In another example, a method of the present disclosure comprises (a) decreasing in a biological sample by affinity depletion N-terminal tau, mid-domain tau, or N-terminal tau and mid-domain tau, wherein the biological sample is a blood sample or a CSF sample; (b) enriching tau that remains after affinity depletion, which may be referred to as N-terminal-independent tau and/or mid-domain-independent tau, by a method that comprises (i) removing additional proteins from the biological sample by protein precipitation and separation of the precipitated proteins to obtain a supernatant, and then purifying tau from the supernatant by solid phase extraction, or (ii) affinity purifying MTBR tau, thereby producing by either (i) or (ii) enriched tau; (c) cleaving the enriched tau with a protease and then optionally desalting the resultant cleavage product by solid phase extraction to obtain a sample comprising proteolytic peptides of tau; and (d) performing liquid chromatography-mass spectrometry (LC/MS) with the sample comprising proteolytic peptides of tau to detect and measure the concentration of at least one proteolytic peptide of tau.

In another example, a method of present disclosure comprises (a) decreasing in a biological sample by affinity depletion N-terminal tau, mid-domain tau, or N-terminal tau and mid-domain tau, wherein the biological sample is a blood sample or a CSF sample; (b) removing additional proteins from the biological sample by protein precipitation and separation of the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography 13 mass spectrometry with the sample comprising proteolytic peptides of tau to detect and measure the concentration at least one proteolytic peptide of tau.

In another example, a method of the present disclosure comprises (a) decreasing in a biological sample by affinity depletion N-terminal tau, mid-domain tau, or N-terminal tau and mid-domain tau, wherein the biological sample is a blood sample or a CSF sample; (b) affinity purifying MTBR tau; (c) cleaving the purified MTBR tau with a protease and then optionally desalting the resultant cleavage product by solid phase extraction to obtain a sample comprising proteolytic peptides of MTBR tau; and (d) performing liquid chromatography-mass spectrometry with the sample comprising proteolytic peptides of MTBR tau to detect and measure the concentration at least one proteolytic peptide of MTBR tau.

In another example, a method of the present disclosure comprises (a) affinity purifying MTBR tau from a biological sample, wherein the biological sample is a blood sample or a CSF sample; (b) cleaving the purified MTBR tau with a protease and then optionally desalting the resultant cleavage product by solid phase extraction to obtain a sample comprising proteolytic peptides of MTBR tau; and (c) performing liquid chromatography-mass spectrometry with the sample comprising proteolytic peptides of MTBR tau to detect and measure the concentration at least one proteolytic peptide of MTBR tau.

The present disclosure further contemplates in each of the above methods determining the presence/absence of one or more protein in the biological sample and/or measuring the concentration of one or more additional protein in the biological sample. In some embodiments, the one or more protein may be a protein depleted from the biological sample prior to purification of tau. For instance, in certain embodiments, N-terminal tau and/or mid-domain tau species may be identified and/or quantified separately from tau species (e.g., MTBR tau, C-terminal tau) quantified by the methods disclosed herein. Alternatively, or in addition, Aβ, ApoE, or any other protein of interest may be identified and/or quantified either by processing a portion of the biological sample in parallel, by depleting the protein of interest from the biological sample prior to utilization in the methods disclosed herein, or by depleting the protein of interest from the biological sample during the sample processing steps disclosed herein.

The biological sample, suitable internal standards, and the steps of depleting one or more protein, purifying tau, cleaving purified tau with a protease, and mass spectrometry are described in more detail below.

Biological Sample

Suitable biological samples include a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing a neurodegenerative disease, a subject with signs and/or symptoms of a neurodegenerative disease, or a subject diagnosed with a neurodegenerative disease. In further embodiments, the neurodegenerative disease may be a tauopathy. In specific examples, the tauopathy may be Alzheimer's disease (AD), progressive supranuclear palsy (PSP), corticobasal degeneration (CBD), or frontotemporal lobar degeneration (FTLD). In other embodiments, the subject is a laboratory animal. In a further embodiment, the subject is a laboratory animal genetically engineered to express human tau and optionally one or more additional human protein (e.g., human Aβ, human ApoE, etc.).

CSF may have been obtained by lumbar puncture with or without an indwelling CSF catheter. Multiple blood or CSF samples contemporaneously collected from the subject may be pooled. Blood may have been collected by veni-puncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to deplete other analytes (e.g. proteins peptides, metabolites).

The size of the sample used can and will vary depending upon the sample type, the health status of the subject from whom the sample was obtained, and the analytes to be analyzed (in addition to tau). CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

(b) Isotope-Labeled, Internal Tau Standard

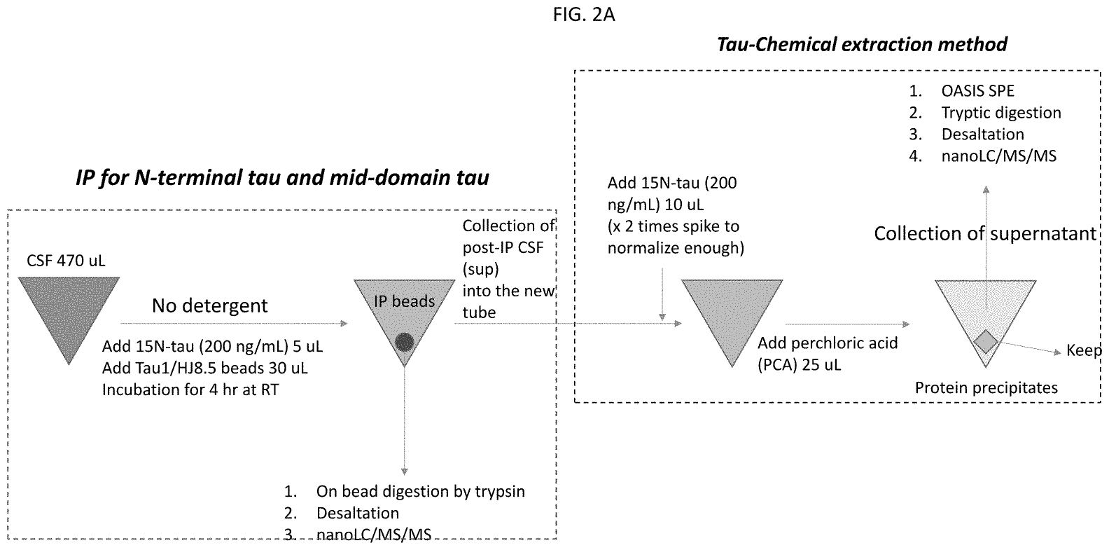

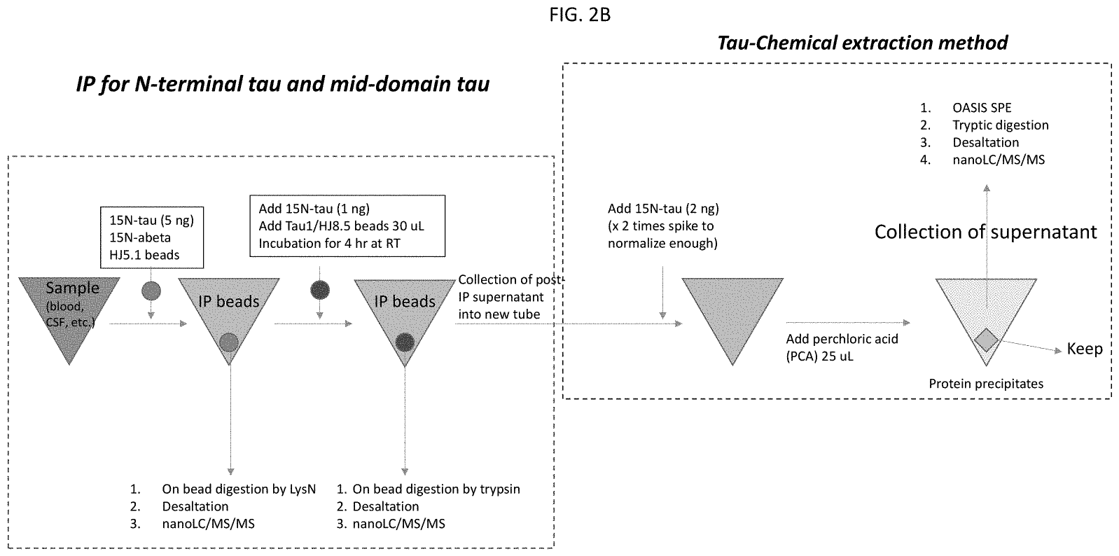

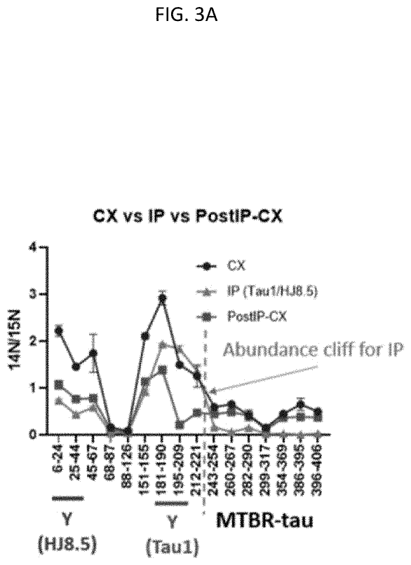

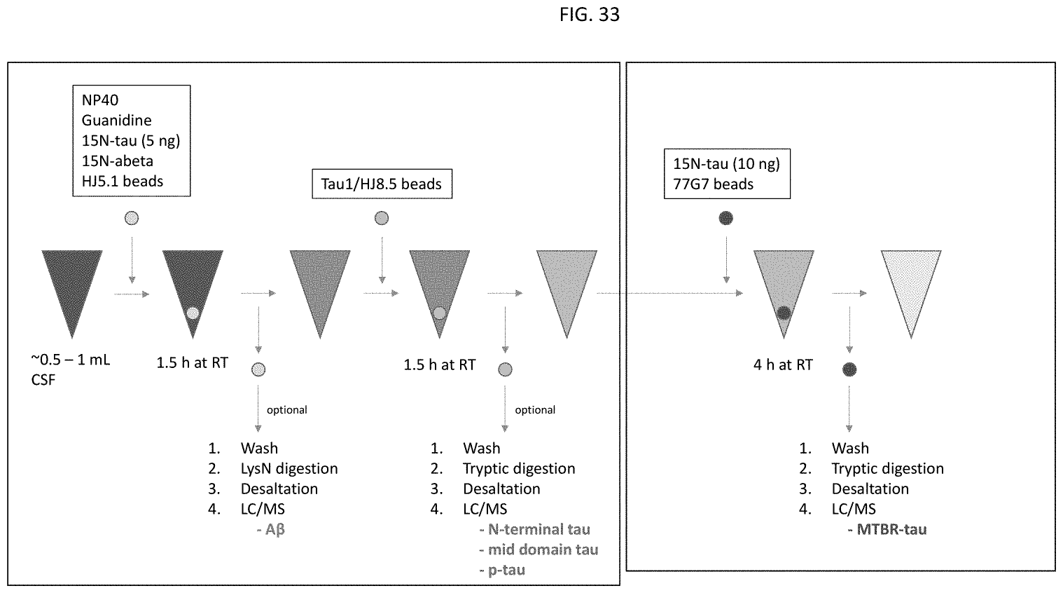

Isotope-labeled tau may be used as an internal standard to account for variability throughout sample processing and optionally to calculate an absolute concentration. Generally, an isotope-labeled, internal tau standard is added before significant sample processing, and it can be added more than once if needed. See, for instance, the methods depicted in FIG. 2 and FIG. 33.

Multiple isotope-labeled internal tau standards are described herein. All have a heavy isotope label incorporated into at least one amino acid residue. One or more full-length isoforms may be used. Alternatively, or in addition, tau isoforms with post-translational modifications and/or peptide fragments of tau may also be used, as is known in the art. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous tau analyte signals. As shown herein, suitable heavy isotope labels include, but are not limited to ²H, ¹³C, and ¹⁵N. Typically, about 1-10 ng of internal standard is usually sufficient.

(c) Depleting One or More Protein

Methods of the present disclosure comprise a step wherein one or more protein is depleted from a sample. The term “deplete” means to diminish in quantity or number. Accordingly, a sample depleted of a protein may have any amount of the protein that is measurably less than the amount in the original sample, including no amount of the protein.

Protein(s) may be depleted from a sample by a method that specifically targets one or more protein, for example by affinity depletion, solid phase extraction, or other method known in the art. Targeted depletion of a protein, or multiple proteins, may be used in situations where downstream analysis of that protein is desired (e.g., identification, quantification, analysis of post-translation modifications, etc.). For instance, Aβ peptides may be identified and quantified by methods known in the art following affinity depletion of Aβ with a suitable epitope-binding agent. As another non-limiting example, apolipoprotein E (ApoE) status may be determined by methods known in the art following affinity depletion of ApoE and identification of the ApoE isoform. Targeted depletion may also be used to isolate other proteins for subsequent analysis including, but not limited to, apolipoprotein J, synuclein, soluble amyloid precursor protein, alpha-2 macroglobulin, S100B, myelin basic protein, an interleukin, TNF, TREM-2, TDP-43, YKL-40, VILIP-1, NFL, prion protein, pNFH, and DJ-1. Targeted depletion of certain tau proteins is also used herein to enrich for other tau proteins and/or eliminate proteins that cofound the mass spectrometry analysis. For instance, in certain embodiments of the present disclosure, N-terminal tau proteins and/or mid-domain tau proteins are depleted from a sample prior to further sample processing for analysis by mass spectrometry. Downstream analysis of the depleted tau proteins may or may not occur, but both options are contemplated by the methods of the present disclosure.

In some embodiments, targeted depletion may occur by affinity depletion. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. The molecule may also be a polymer or other material that selectively absorbs a protein of interest. As a non-limiting example, polyhydroxymethylene substituted by fat oxethylized alcohol (e.g., PHM-L LIPOSORB, Sigma Aldrich) may be used to selectively absorb lipoproteins (including ApoE) from serum. Two or more affinity depletion agents may be combined to sequentially or simultaneously deplete multiple proteins.

In some embodiments, a method of the present disclosure comprises affinity depleting one or more protein from a sample using at least one epitope-binding agent that specifically binds to an epitope within amino acids 1 to 243 of tau-441, inclusive (or within a similarly defined region for 0N or 1N isoforms). In various embodiments, one, two, three or more epitope-binding agents may be used. When two or more epitope-binding agents are used, they may be used sequentially or simultaneously.

In some embodiments, a method of the present disclosure comprises affinity depleting one or more protein from a sample using an epitope-binding agent that specifically binds to an epitope within the N-terminus of tau (e.g., amino acids 1 to 103 of tau-441, inclusive), and an epitope-binding agent that specifically binds to an epitope within the mid-domain of tau (e.g., amino acids 104 to 243 of tau-441, inclusive). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive, and an epitope-binding agent that specifically binds to an epitope within amino acids 104 to 243 of tau-441, inclusive (or within similarly defined regions for 0N or 1N isoforms). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or 1N isoforms); an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or 1N isoforms); an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or 1N isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or 1N isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In each of the above embodiments, the epitope binding agent may comprise an antibody or an aptamer. In some embodiments, the epitope-binding agent that specifically binds to amyloid beta is HJ5.1, or is an epitope-binding agent that binds the same epitope as HJ5.1 and/or competitively inhibits HJ5.1. In some embodiments, the epitope-binding agent that specifically binds to that specifically binds to an epitope within amino acids 1 to 103 of tau-441, inclusive, is HJ8.5, or is an epitope-binding agent that binds the same epitope as HJ8.5 and/or competitively inhibits HJ8.5. In some embodiments, the epitope-binding agent that specifically binds to that specifically binds to an epitope within amino acids 104 to 221 of tau-441, inclusive, is Tau1, or is an epitope-binding agent that binds the same epitope as Tau1 and/or competitively inhibits Tau1. Methods for identifying epitopes to which an antibody specifically binds, and assays to evaluate competitive inhibition between two antibodies, are known in the art.

Alternatively, protein(s) may be depleted from a sample by a more general method, for example by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides (e.g., tau, Aβ, etc.).

In some embodiments, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include perchloric acid, trichloroacetic acid, acetonitrile, methanol, and the like. In an exemplary embodiment, proteins are depleted from a sample by acid precipitation. In a further embodiment, proteins are depleted from a sample by acid precipitation using perchloric acid.

As a non-limiting example, proteins may be depleted from a sample by acid precipitation using perchloric acid. As used herein, “perchloric acid” refers to 70% perchloric acid unless otherwise indicated. In some embodiments, perchloric acid is added to a final concentration of about 1% v/v to about 15% v/v. In other embodiments, perchloric acid is added to a final concentration of about 1% v/v to about 10% v/v. In other embodiments, perchloric acid is added to a final concentration of about 1% v/v to about 5% v/v. In other embodiments, perchloric acid is added to a final concentration of about 3% v/v to about 15% v/v. In other embodiments, perchloric acid is added to a final concentration of about 3% v/v to about 10% v/v. In other embodiments, perchloric acid is added to a final concentration of about 3% v/v to about 5% v/v. In other embodiments, perchloric acid is added to a final concentration of 3.5% v/v to about 15% v/v, 3.5% v/v to about 10% v/v, or 3.5% v/v to about 5% v/v. In other embodiments, perchloric acid is added to a final concentration of about 3.5% v/v. Following addition of the perchloric acid, the sample is mixed well (e.g., by a vortex mixer) and held at a cold temperature, typically for about 10 minutes or longer, to facilitate precipitation. For example, samples may be held for about 10 minutes to about 60 minutes, about 20 minutes to about 60 minutes, or about 30 minutes to about 60 minutes. In other example, samples may be held for about 15 minutes to about 45 minutes, or about 30 minutes to about 45 minutes. In other examples, samples may be held for about 15 minutes to about 30 minutes, or about 20 minutes to about 40 minutes. In other examples, samples are held for about 30 minutes. The sample is then centrifuged at a cold temperature to pellet the precipitated protein, and the supernatant (i.e., the acid soluble fraction), comprising soluble tau, is transferred to a fresh vessel. As used in the above context, a “cold temperature” refers to a temperature of 10° C. or less. For instance, a cold temperature may be about 1° C., about 2° C., about 3° C., about 4° C., about 5° C., about 6° C., about 7° C., about 8° C., about 9° C., or about 10° C. In some embodiments, a narrower temperature range may be preferred, for example, about 3° C. to about 5° C., or even about 4° C. In certain embodiments, a cold temperature may be achieved by placing a sample on ice.

Two or more methods from one or both of the above approaches may be combined to sequentially or simultaneously deplete multiple proteins. For instance, one or more proteins may be selectively depleted (targeted depletion) followed by depletion of high abundance/molecular weight proteins. Alternatively, high abundance/molecular weight proteins may be first depleted followed by targeted depletion of one or more proteins. In still another alternative, high abundance/molecular weight proteins may be first depleted followed by a first round of targeted depletion of one or more proteins and then a second round of targeted depletion of one or more different protein(s) than targeted in the first round. Other iterations will be readily apparent to a skilled artisan.

(d) Purifying Tau

Another step of the methods disclosed herein comprises purifying tau, in particular MTBR tau. In some examples, the MTBR tau is N-terminal-independent and/or mid-domain-independent MTBR tau. The purified tau may be partially purified or completely purified.

In some embodiments, a method of the present disclosure comprises purifying tau by solid phase extraction. Purifying tau by solid phase extraction comprises contacting a sample comprising tau with a solid phase comprising a sorbent that adsorbs tau, one or more wash steps, and elution of tau from the sorbent. Suitable sorbents include reversed-phase sorbents. Suitable reversed phase sorbents are known in the art and include, but are not limited to alkyl-bonded silicas, aryl-bonded silicas, styrene/divinylbenzene materials, N-vinylpyrrolidone/divinylbenzene materials. In an exemplary embodiment, the reversed phase material is a polymer comprising N-vinylpyrrolidone and divinylbenzene or a polymer comprising styrene and divinylbenzene. In an exemplary embodiment, a sorbent is Oasis HLB (Waters). Prior to contact with the supernatant comprising tau, the sorbent is typically preconditioned per manufacturer's instructions or as is known in the art (e.g., with a water miscible organic solvent and then the buffer comprising the mobile phase). In addition, the supernatant may be optionally acidified, as some reversed-phase materials retain ionized analytes more strongly than others. The use of volatile components in the mobile phases and for elution is preferred, as they facilitate sample drying. In exemplary embodiments, a wash step may comprise the use of a liquid phase comprising about 0.05% v/v trifluoroacetic acid (TFA) to about 1% v/v TFA, or an equivalent thereof. In some examples, the wash may be with a liquid phase comprising about 0.05% v/v to about 0.5% v/v TFA or about 0.05% v/v to about 0.1% v/v TFA. In some examples, the wash may be with a liquid phase comprising about 0.1% v/v to about 1.0% v/v TFA or about 0.1% v/v to about 0.5% v/v TFA. Bound tau is then eluted with a liquid phase comprising about 20% v/v to about 50% v/v acetonitrile (ACN), or an equivalent thereof. In some examples, tau is may be eluted with a liquid phase comprising about 20% v/v to about 40% v/v ACN, or about 20% v/v to about 30% v/v ACN. In some examples, tau is may be eluted with a liquid phase comprising about 30% v/v to about 50% v/v ACN, or about 30% v/v to about 40% v/v ACN. The eluate may be dried by methods known in the art (e.g., vacuum drying (e.g., speed-vac), lyophilization, evaporation under a nitrogen stream, etc.).

In some embodiments, a method of the present disclosure comprises purifying MTBR tau by affinity purification. Affinity purification refers to methods that enrich for a protein of interest by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. Purifying MTBR tau by affinity purification comprises contacting a sample comprising tau with a suitable immobilized ligand, one or more wash steps, and elution of MTBR tau from the immobilized ligand.

In some embodiments, a method of the present disclosure comprises purifying MTBR tau by affinity purification using at least one epitope-binding agent that specifically binds to an epitope within amino acids 235 to 368 of tau-441, inclusive, or within amino acids 244 to 368 of tau-441, inclusive (or within similarly defined regions for other full-length isoforms). In various embodiments, one, two, three or more epitope-binding agents may be used. When two or more epitope-binding agents are used, they may be used sequentially or simultaneously. Non-limiting examples of suitable epitope-binding agents include antibodies 77G7, RD3, RD4, UCB1017, and PT76 described in Vandermeeren et al., J Alzheimers Dis, 2018, 65:265-281, and antibodies E2814 and 7G6 described in Roberts et al., Acta Neuropathol Commun, 2020, 8: 13, as well as other epitope-binding agents that specifically bind the same epitopes as those antibodies. In further embodiments, a method of the present disclosure comprises purifying MTBR tau by affinity purification using an epitope-binding agent that specifically binds to an epitope within R1 of MTBR tau, an epitope-binding agent that specifically binds to an epitope within R2 of MTBR tau, an epitope-binding agent that specifically binds to an epitope within R3 of MTBR tau, an epitope-binding agent that specifically binds to an epitope within R4 of MTBR tau, an epitope-binding agent that specifically binds to an epitope unique to 3R tau, an epitope-binding agent that specifically binds to an epitope unique to 4R tau, an epitope-binding agent that specifically binds to an epitope spanning R1 and R2 of MTBR tau, an epitope-binding agent that specifically binds to an epitope spanning R2 and R3 of MTBR tau, an epitope-binding agent that specifically binds to an epitope spanning R3 and R4 of MTBR tau, or any combination thereof. In a specific example, a method of the present disclosure comprises purifying MTBR tau by affinity purification using an epitope-binding agent that specifically binds to an epitope comprising amino acids 316 to 355 of tau-441 (or the same region for the other full length isoforms). In various embodiments, one, two, three or more epitope-binding agents may be used. When two or more epitope-binding agents are used, they may be used sequentially or simultaneously.

In each of the above embodiments, the epitope-binding agent may comprise an antibody or an aptamer. In some embodiments, an epitope-binding agent that specifically binds to an epitope within R3 and R4 of MTBR tau is 77G7, or is an epitope-binding agent that binds the same epitope as 77G7 and/or competitively inhibits 77G7 (BioLegend). In some embodiments, an epitope-binding agent that specifically binds to an epitope unique to 3R tau is RD3 (de Silva et al., Neuropathology and Applied Neurobiology, 2003, 29: 288-302), or is an epitope-binding agent that binds the same epitope as RD3 and/or competitively inhibits RD3. In some embodiments, an epitope-binding agent that specifically binds to an epitope unique to 4R tau is RD4 (de Silva et al., Neuropathology and Applied Neurobiology, 2003, 29: 288-302), or is an epitope-binding agent that binds the same epitope as RD4 and/or competitively inhibits RD4.

(e) Cleaving Purified Tau with a Protease

Another step of the methods disclosed herein comprises cleaving purified tau with a protease. Cleaving purified tau with a protease comprises contacting a sample comprising purified tau with a protease under conditions suitable to digest tau. When affinity purification is used, digestion may occur after eluting tau from the immobilized ligand or while tau is bound. Suitable proteases include but are not limited to trypsin, Lys-N, Lys-C, and Arg-N. In a preferred embodiment, the protease is trypsin. The resultant cleavage product is a composition comprising proteolytic peptides of tau. When the protease is trypsin, the resultant cleavage product comprises tryptic peptides of tau. Following proteolytic cleavage, the resultant cleavage product is typically desalted by solid phase extraction.
(f) LC-MS

Another step of the methods disclosed herein comprises performing liquid chromatography-mass spectrometry (LC-MS) with a sample comprising proteolytic peptides of tau to detect and measure the concentration of at least one proteolytic peptide of tau. Thus, in practice, the disclosed methods use one or more proteolytic peptide of tau to detect and measure the amount of tau protein present in the biological sample.

In embodiments where trypsin is the protease, proteolytic peptides of tau that indicate the presence of MTBR tau include but are not limited to the peptides listed in Table A. When using an alternative enzyme for digestion, the resulting proteolytic peptides may differ slightly but can be readily determined by a person of ordinary skill in the art. Without wishing to be bound by theory, it is believed that a variation in the amount of a tryptic peptide between two biological samples of the same type reflects a difference in the MTBR tau species that make up those biological samples. As disclosed herein, the amounts of certain proteolytic peptides of MTBR tau, as well ratios of certain proteolytic peptides of MTBR tau, may provide clinically meaningful information to guide treatment decisions. Thus, methods that allow for detection and quantification of tryptic peptides of MTBR tau have utility in the diagnosis and treatment of many neurodegenerative diseases.

TABLE A
Tryptic peptides of tau that indicate
the presence of MTBR tau

| Tryptic peptide | | |
|---|---|---|
| name(s) | Amino acid sequence | SEQ ID NO: |
| IGST | IGSTENLK | 2 |
| LQTA | LQTAPVPMPDLK | 3 |
| VQII | VQIINK | 4 |
| LDLS | LDLSNVQSK | 5 |
| HVPG | HVPGGGSVQIVYKPVDLSK | 6 |
| IGSL | IGSLDNITHVPGGGNK | 7 |
| tau368 | IGSLDNITHVPGGGN | 8 |
| VQIV | VQIVYKPVDLSK | 9 |

Proteolytic peptides of tau may be separated by a liquid chromatography system interfaced with a high-resolution mass spectrometer. Suitable LC-MS systems may comprise a <1.0 mm ID column and use a flow rate less than about 100 μl/min. In preferred embodiments, a nanoflow LC-MS system is used (e.g., about 50-100 μm ID column and a flow rate of <1 μL/min, preferably about 100-800 nL/min, more preferably about 200-600 nL/min). In an exemplary embodiment, an LC-MS system may comprise a 0.05 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., AGC setting using an orbitrap) and/or injection time. In an exemplary embodiment, a mass spectrometry protocol outlined in the Examples is used.

### III. Uses of MTBR Tau Measurements

The present disclosure also encompasses the use of measurements of MTBR tau species, in particular mid-domain-independent MTBR tau species, in blood or CSF as biomarkers of pathological features and/or clinical symptoms of tauopathies in order to diagnose, stage, choose treatments appropriate for a given disease stage, and modify a given treatment regimen (e.g., change a dose, switch to a different drug or treatment modality, etc.). The pathological feature may be an aspect of tau pathology (e.g., amount of tau deposition, presence/absence of a post-translational modification, amount of a post-translation modification, etc.). Alternatively, or in addition to tau deposition, a pathological feature may be tau-independent. For instance, amyloid beta (Aβ) deposition in the brain or in arteries of the brain when the tauopathy is Alzheimer's disease. The clinical symptom may be dementia, as measured by a clinically validated instrument (e.g., MMSE, CDR-SB, etc.), or any other clinical symptom associated with the tauopathy. Also contemplated is the use of measurements of MTBR tau species, in particular mid-domain-independent MTBR tau species, in blood or CSF as biomarkers of other pathological features and clinical symptoms known in the art for 3R- and 4R-tauopathies. Advantageously, MTBR tau species, including but not limited to mid-domain-independent MTBR tau species, not only discriminate a disease state from a healthy state, but also discriminate between the various tauopathies.

Accordingly, in one aspect, the present disclosure provides a method for measuring tauopathy-related pathology in a subject, the method comprising quantifying one or more MTBR tau species in a biological sample obtained from a subject, such as a blood sample or a CSF sample, wherein the amount(s) of the quantified MTRB-tau species is/are a representation of tauopathy-related pathology in the brain of the subject. The tauopathy may be a 3R-tauopathy, a mixed 3R/4R-tauopathy, or a 4R-tauopathy. The disease-related pathology may be tau deposition, tau post-translational modification, amyloid plaques in the brain and/or arteries of the brain, or other pathological feature known in the art. The subject may or may not have clinical symptoms of the tauopathy. In preferred embodiments, at least one MTBR tau species quantified is a mid-domain-independent MTBR tau species. In further embodiments, two or more MTBR tau species quantified are mid-domain-independent MTBR tau species. In still further embodiments, each MTBR tau species quantified is a mid-domain-independent MTBR tau species.

In another aspect, the present disclosure provides a method for diagnosing a tauopathy in a subject, the method comprising quantifying one or more MTBR tau species in a biological sample obtained from a subject, such as a blood sample or a CSF sample, and diagnosing a tauopathy when the quantified MTBR tau species is/are about 1.5σ or above, where σ is the standard deviation defined by the normal distribution measured in a control population that does not have clinical signs or symptoms of a tauopathy and is amyloid negative as measured by PET imaging and/or Aβ42/40 measurement in CSF. The tauopathy may be a 3R-tauopathy, a mixed 3R/4R-tauopathy, or a 4R-tauopathy. The subject may or may not have clinical symptoms of disease. In preferred embodiments, at least one MTBR tau species quantified is a mid-domain-independent MTBR tau species. In further embodiments, two or more MTBR tau species quantified are mid-domain-independent MTBR tau species. In still further embodiments, each MTBR tau species quantified is a mid-domain-independent MTBR tau species.

In another aspect, the present disclosure provides a method for measuring tauopathy disease stability in a subject, the method comprising quantifying one or more MTBR tau species in a first biological sample obtained from a subject and then in a second biological sample obtained from the same subject at a later time (e.g., weeks, months or years later), and calculating the difference between the quantified MTBR tau species between the samples, wherein a statistically significant increase in the quantified MTBR tau species in the second sample indicates disease progression, a statistically significant decrease in the quantified MTBR tau species in the second sample indicates disease improvement, and no change indicates stable disease. The tauopathy may be a 3R-tauopathy, a mixed 3R/4R-tauopathy, or a 4R-tauopathy. The subject may or may not have clinical symptoms of disease, and may or may not be receiving a tau therapy. In some examples, a tau therapy is administered one or more times to the subject in the period of time between collection of the first and second biological sample, and the measure of disease stability is an indication of the effectiveness, or lack thereof, of the tau therapy. In preferred embodiments, at least one MTBR tau species quantified is a mid-domain-independent MTBR tau species. In further embodiments, two or more MTBR tau species quantified are mid-domain-independent MTBR tau species. In still further embodiments, each MTBR tau species quantified is a mid-domain-independent MTBR tau species.

In another aspect, the present disclosure provides a method for treating a subject with a tauopathy, the method comprising quantifying one or more MTBR tau species in a biological sample obtained from a subject, such as a blood sample or a CSF sample; and providing a tau therapy to the subject to improve a measurement of disease-related pathology or a clinical symptom, wherein the subject has a quantified MTBR tau species at least 1 standard deviation, preferably at least 1.3 standard deviations, more preferably at least 1.5 standard deviations or even more preferably at least 2 standard deviations, above or below the mean (i.e., differs by 1σ, 1.3σ, 1.5σ, or 1.5σ, respectively, where a is the standard deviation defined by the normal distribution measured in a control population does not have clinical signs or symptoms of a tauopathy and that is amyloid negative as measured by PET imaging and/or Aβ42/40 measurement in CSF. In addition to using a threshold (e.g. at least 1 standard deviation above or below the mean), in some embodiments the extent of change above or below the mean may be used as criteria for treating a subject. The tauopathy may be a 3R-tauopathy, a mixed 3R/4R-tauopathy, or a 4R-tauopathy. The measurement of disease-related pathology may be tau deposition as measured by PET imaging, tau post-translational modification as measured by mass spectrometry or other suitable method, amyloid plaques in the brain or arteries of the brain as measured by PET imaging, amyloid plaques as measured by Aβ42/40 in CSF, or other pathological features known in the art. The clinical symptom may be dementia, as measured by a clinically validated instrument (e.g., MMSE, CDR-SB, etc.) or other clinical symptoms known in the art for 3R- and 4R-tauopathies. In preferred embodiments, at least one MTBR tau species quantified is a mid-domain-independent MTBR tau species. In further embodiments, two or more MTBR tau species quantified are mid-domain-independent MTBR tau species. In still further embodiments, each MTBR tau species quantified is a mid-domain-independent MTBR tau species. Many tau therapies target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation (general antagonism of tau or antagonism of a specific tau isoform), or increase NFT clearance; a variety of therapies are designed to reduce CNS inflammation or brain insulin resistance; etc. However, not all tauopathies share the same pathophysiological changes. Therefore, the efficacy of these various tau therapies can be improved by administering them to subjects that are correctly identified as having a 3R-tauopathy, a mixed 3R/4R-tauopathy, or a 4R-tauopathy.

The term “mid-domain-independent MTBR tau” refers to a plurality of MTBR tau species that lack all or substantially all of the mid-domain region of tau, and therefore also the N-terminus region. These mid-domain-independent MTBR tau species remain after tau species comprising mid-domain tau have been depleted, partially or completely, from a biological sample, preferably from a blood or CSF sample. Suitable biological samples are described in Section II(a), the disclosures of which are incorporated into this section by reference. Depletion of mid-domain tau may occur by targeted depletion of these tau species, for example by affinity depletion using an epitope-binding agent that specifically binds to an epitope within the N-terminus or mid-domain of tau. Multiple epitope-binding agents may also be used—for example, a first epitope-binding agent that specifically binds to an epitope within the N-terminus of tau and a second epitope-binding agent that specifically binds to an epitope within the mid-domain of tau. Further details can be found in Section II(c), the disclosures of which are incorporated into this section by reference. Typically, at least 50% (e.g., 50%, 60%, 70%, 80%, 90% or more) of the targeted protein in the starting material is depleted. In some embodiments, about 70% or more, about 80% or more, or about 90% or more of the targeted protein in the starting material is depleted. After depletion of mid-domain tau from a biological sample, steps can be taken (1) to enrich the remaining tau species, which will include mid-domain-independent MTBR tau, for example by removing others proteins by precipitation and/or purifying tau proteins by solid phase extraction, or (2) to selectively enrich mid-domain-independent MTBR tau species, for example by affinity purification using an epitope-binding agent that specifically binds to an epitope within the MTBR. The term “enrich” means to increase in quantity or number. Further details can be found in Section II, the disclosures of which are incorporated into this section by reference. Preferably, mid-domain-independent MTBR tau species are enriched at least 100-fold over their amount in the CSF. In some examples, mid-domain-independent MTBR tau species may be enriched about 100-fold to about 1000-fold—for instance, about 100-fold, about 200-fold, about 300-fold, about 400-fold, about 500-fold, about 600-fold, about 700-fold, about 800-fold, about 900-fold, about 1000-fold. In some examples, mid-domain-independent MTBR tau species may be enriched about 500-fold to about 1000-fold, or even more. MTBR tau can be quantified in processed CSF or blood samples obtained from a subject, wherein the CSF or blood samples are depleted of mid-domain tau and then enriched for MTBR tau by LC-MS, as described in Section II or the Examples, or by other methods known in the art (e.g., multiplexed assays (such as xMAP technology by Luminex, single molecule protein detection (such as Simoa® bead technology), and the like). In embodiments where mid-domain tau is not depleted from a biological sample, tau is still typically enriched by methods as described above and to the extent described above.

In each of the above aspects, suitable MTBR tau species may include, but are not limited to, MTBR tau species and/or mid-domain-independent MTBR species comprising the amino sequence of SEQ ID NO: 2 (IGSTENLK), SEQ ID NO: 3 (LQTAPVPMPDLK), SEQ ID NO: 4 (VQIINK), SEQ ID NO: 5 (LDLSNVQSK), SEQ ID NO: 6 (HVPGGGSVQIVYKPVDLSK), SEQ ID NO: 8 (IGSLDNITHVPGGGN), SEQ ID NO: 9 (VQIVYKPVDLSK), or combinations thereof. The choice of MTBR species to measure may depend on the intended purpose of the method. For instance, when the tauopathy is a 3R-tauopathy, MTBR tau species comprising SEQ ID NO: 9 (VQIVYKPVDLSK) may be decreased as compared to a mixed 3R/4R-tauopathy or a 4R-tauopathy, while MTBR tau species comprising SEQ ID NO: 2 (IGSTENLK), SEQ ID NO: 4 (VQIINK), SEQ ID NO: 5 (LDLSNVQSK), SEQ ID NO: 6 (HVPGGGSVQIVYKPVDLSK), and/or SEQ ID NO: 8 (IGSLDNITHVPGGGN) may be unchanged or increased as compared to other tauopathies. Conversely, when the tauopathy is a 4R-tauopathy, MTBR species comprising SEQ ID NO: 9 (VQIVYKPVDLSK) may be increased as compared to a mixed 3R/4R-tauopathy or a 3R-tauopathy, while MTBR tau species comprising SEQ ID NO: 2 (IGSTENLK), SEQ ID NO: 4 (VQIINK), SEQ ID NO: 5 (LDLSNVQSK), SEQ ID NO: 6 (HVPGGGSVQIVYKPVDLSK) and/or SEQ ID NO: 8 (IGSLDNITHVPGGGN) may be unchanged or decreased as compared to other tauopathies. As an additional example, 4R tauopathies may be discriminated from AD by quantifying MTBR tau species comprising SEQ ID NO: 2 (IGSTENLK), SEQ ID NO: 4 (VQIINK), SEQ ID NO: 5 (LDLSNVQSK), SEQ ID NO: 6 (HVPGGGSVQIVYKPVDLSK), and/or SEQ ID NO: 8 (IGSLDNITHVPGGGN). The use of mid-domain-independent MTBR tau species can boost the discrimination power, which may be further boosted by using a ratio of two different mid-domain-independent MTBR tau species. For instance, when the tauopathy is a 3R-tauopathy or a mixed 3R/4R-tauopathy, ratios of SEQ ID NO: 3 to SEQ ID NO: 6, SEQ ID NO: 3 to SEQ ID NO: 8, or SEQ ID NO: 6 to SEQ ID NO: 8 may be used. When the tauopathy is a 4R-tauopathy, ratios of SEQ ID NO: 2, 4, 5, or 9 to SEQ ID NO: 6, 7 or 8 may be used. Mathematical operations other than a ratio may also be used.

Exemplary uses of mid-domain-independent MTBR tau-243 can serve to illustrate various aspects discussed above, but such discussions do not limit the scope of the invention. “Mid-domain-independent MTBR tau-243” is described in detail in Example 3. It has the amino acid sequence of SEQ ID NO: 3 (LQTAPVPMPDLK), and is a tryptic peptide of a plurality of mid-domain-independent MTBR tau species that all comprise the amino acid sequence of SEQ ID NO: 3. Measuring the amount of mid-domain-independent MTBR tau-243 is one means by which to measure, in a given sample, the amount of this specific group of mid-domain-independent MTBR tau species. As shown in Examples 2 and 3, increases in the amount of CSF mid-domain-independent MTBR tau-243 recapitulate direct measures of increasing Aβ deposition and tau deposition in the brain associated with Alzheimer's disease (AD). Stated another way, the amount of CSF mid-domain-independent MTBR tau-243 (and therefore the amount of CSF mid-domain-independent MTBR tau comprising SEQ ID NO: 3) is a representation of AD-related pathology (e.g., tau deposition in the brain, Aβ deposition in the brain, etc.). These amounts can therefore be used to measure AD-related pathology, to determine a subject's amyloid status, and to diagnose AD in subjects without clinical symptoms of the disease. The amount of CSF mid-domain-independent MTBR tau-243 also recapitulates changes measured during clinical stages of AD, for example as defined by the results of MMSE or CDR-SB testing. Accordingly, the amount of mid-domain-independent MTBR tau-243 (and therefore the amount of mid-domain-independent MTBR tau comprising SEQ ID NO: 3) can also be used to diagnose and stage AD in subjects across the entire disease spectrum (e.g., pre-clinical to clinical). A utility for diagnosing and staging AD in subjects across the entire disease spectrum was not observed for every tryptic peptide of mid-domain-independent MTBR tau. See, for example, mid-domain-independent MTBR tau-299 and mid-domain-independent MTBR tau-354 data in Example 3. This demonstrates that the group of peptides which make-up “mid-domain-independent MTBR tau comprising SEQ ID NO: 3” can be different than the group of peptides that make-up “mid-domain-independent MTBR tau comprising SEQ ID NO: 6” (though there may be overlap), and supports the use of the abundance of SEQ ID NO: 3 (among others) as a disease-specific biomarker for AD (and potentially other tauopathies independent of the method by which it is measured (e.g., mass spectrometry, ELISA, etc.). After diagnosing and/or staging disease, treatments may then be provided to the subject to decrease, or prevent any further increase, in the amount of mid-domain-independent MTBR tau-243 in CSF and/or to decrease, or prevent any further increase, of another clinical sign or symptom of AD. Choice of treatment may be further guided by knowledge of the specific disease stage that is informed by the amount of mid-domain-independent MTBR tau-243—for instance, therapies designed to prevent Aβ deposition, reverse Aβ deposition, prevent tau deposition, reverse tau deposition, and improve clinical signs of disease would be used in subjects with different, albeit potentially overlapping, amount of mid-domain-independent MTBR tau-243. The Examples further show that while CSF mid-domain-independent MTBR tau-243 is very useful as a biomarker of AD, it is not as useful for non-AD tauopathies. Non-AD tauopathies can be discriminated by quantifying mid-domain-independent MTBR tau species comprising SEQ ID NO: 2 (IGSTENLK), SEQ ID NO: 4 (VQIINK), SEQ ID NO: 5 (LDLSNVQSK), SEQ ID NO: 6 (HVPGGGSVQIVYKPVDLSK) or SEQ ID NO: 8 (IGSLDNITHVPGGGN), and their ratios. Although the Examples demonstrate the above principles with CSF samples, blood samples are contemplated as suitable alternatives.

In a specific embodiment, the present disclosure provides a method for measuring Alzheimer disease (AD)-related pathology in a subject, the method comprising providing a processed CSF or blood sample obtained from a subject, wherein the CSF or blood sample is depleted of mid-domain tau and enriched for MTBR tau; and quantifying, in the processed sample, MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof, wherein the amount of the quantified MTRB-tau species, or their ratios, is a representation of AD-related pathology in a brain of a subject.

In another specific embodiment, the present disclosure provides a method for measuring Alzheimer disease (AD)-related tau deposition in a brain of a subject, the method comprising providing a processed CSF or blood sample obtained from a subject, wherein the processed CSF or blood sample is depleted of mid-domain tau and enriched for MTBR tau; and quantifying, in the processed sample, MTBR tau species comprising the amino sequence SEQ ID NO: 3 (LQTAPVPMPDLK) in the processed CSF or blood sample, wherein the amount of the quantified MTRB-tau species is a representation of AD-related tau deposition in a brain of a subject.

In another specific embodiment, the present disclosure provides a method for measuring Alzheimer disease (AD)-related tau deposition in a brain of a subject, the method comprising providing a processed CSF or blood sample obtained from a subject, wherein the CSF or blood sample is depleted of mid-domain tau and enriched for MTBR tau; and quantifying, in the processed sample, MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof, wherein the amount of the quantified MTRB-tau species, or their ratios, is a representation of AD-related tau deposition in a brain of a subject.

In another specific embodiment, the present disclosure provides a method for determining a subject's amyloid status, the method comprising providing a processed CSF or blood sample obtained from a subject, wherein the CSF or blood sample is depleted of mid-domain tau and enriched for MTBR tau; and quantifying, in the processed sample, MTBR tau species comprising the amino sequence of SEQ ID NO: 6 (HVPGGGSVQIVYKPVDLSK), wherein the amount of the quantified MTRB-tau species is a representation of AD-related amyloid beta deposition in a brain of a subject and predicts amyloid-positivity as determined by PIB-PET, for instance by PiB-PET SUVR as described in Ann Neurol 2016; 80:379-387.

In another specific embodiment, the present disclosure provides a method for diagnosing Alzheimer's disease, the method comprising providing a processed CSF or blood sample obtained from a subject, wherein the CSF or blood sample is depleted of mid-domain tau and enriched for MTBR tau; and quantifying, in the processed sample, MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof; and diagnosing Alzheimer's disease when the quantified MTBR tau species differs by about 1.5σ or more, where σ is the standard deviation defined by the normal distribution measured in a control population does not have clinical signs or symptoms of a tauopathy and that is amyloid negative as measured by PET imaging (for instance by PiB-PET SUVR as described in Ann Neurol 2016; 80:379-387) and/or Aβ42/40 measurement in CSF (for instance, a cutoff value for CSF Aβ42/40 calculated from PiB-PET SUVR (Ann Neurol 2016; 80:379-387) that maximizes sensitivity %+Specificity %).

In another specific embodiment, the present disclosure provides a method for measuring Alzheimer disease (AD) progression in a subject, the method comprising providing a first processed CSF or blood sample and a second processed CSF or blood sample, wherein each processed sample is obtained from a single subject, and each processed sample is depleted of mid-domain tau and enriched for MTBR tau; and for each processed sample, quantifying MTBR tau species comprising the amino sequence of ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof; and calculating the difference between the quantified MTBR tau species in the second sample and the first sample, wherein a statistically significant increase in the quantified MTBR tau species in the second sample indicates progression of the subject's Alzheimer's disease.

In another specific embodiment, the present disclosure provides a method for discriminating a 4R-tauopathy, the method comprising providing a processed CSF or blood sample obtained from a subject, wherein the CSF or blood sample is depleted of mid-domain tau and enriched for MTBR tau; and quantifying, in the processed sample, (i) MTBR tau species comprising the amino sequence of SEQ ID NO: 9 (VQIVYKPVDLSK), and (ii) MTBR tau species comprising the amino sequence of SEQ ID NO: 7 (IGSLDNITHVPGGGNK) or MTBR tau species comprising the amino sequence of SEQ ID NO: 8 (IGSLDNITHVPGGGN); wherein the ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising providing a processed CSF or blood sample obtained from a subject, wherein the CSF or blood sample is depleted of mid-domain tau and enriched for MTBR tau; and quantifying, in the processed sample, (i) MTBR tau species comprising the amino sequence of SEQ ID NO: 9 (VQIVYKPVDLSK), and (ii) MTBR tau species comprising the amino sequence of SEQ ID NO: 4 (VQIINK), MTBR tau species comprising the amino sequence of SEQ ID NO: 5 (LDLSNVQSK), MTBR tau species comprising the amino acid sequence of SEQ ID NO: 6 (HVPGGGSVQIVYKPVDLSK), or any combination thereof; wherein a ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising providing a processed CSF or blood sample obtained from a subject, wherein the CSF or blood sample is (a) depleted of N-terminal tau and mid-domain tau, and (b) enriched for MTBR tau; and quantifying, in the processed sample, (i) MTBR tau species comprising the amino sequence of SEQ ID NO: 2 (IGSTENLK), MTBR tau species comprising the amino sequence of SEQ ID NO: 4 (VQIINK), MTBR tau species comprising the amino sequence of SEQ ID NO: 5 (LDLSNVQSK), or combinations thereof, and (ii) MTBR tau species comprising the amino acid sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (i) and (ii) discriminates a 4R-tauopathy from Alzheimer's Disease and a healthy state.

In another specific embodiment, the present disclosure provides a method for discriminating a 4R-tauopathy, the method comprising providing a processed CSF or blood sample obtained from a subject, wherein the CSF or blood sample is (a) depleted of N-terminal tau and mid-domain tau, and (b) enriched for MTBR tau; and quantifying, in the processed sample, (i) MTBR tau species comprising the amino sequence of SEQ ID NO: 2 (IGSTENLK), MTBR tau species comprising the amino sequence of SEQ ID NO: 4 (VQIINK), MTBR tau species comprising the amino sequence of SEQ ID NO: 5 (LDLSNVQSK), or combinations thereof, and (ii) MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (i) and (ii) discriminates a 4R-tauopathy from Alzheimer's Disease and a healthy state.

In another specific embodiment, the present disclosure provides a method for discriminating a 4R-tauopathy, the method comprising providing a processed CSF or blood sample obtained from a subject, wherein the CSF or blood sample is (a) depleted of N-terminal tau and mid-domain tau, and (b) enriched for MTBR tau; and quantifying, in the processed sample, (a) MTBR tau species comprising the amino sequence of SEQ ID NO: 6 (HVPGGGSVQIVYKPVDLSK), and (b) MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (a) and (b) discriminates a 4R-tauopathy from other tauopathies and a healthy state.

In another specific embodiment, the present disclosure provides a method for discriminating a 3R-tauopathy, the method comprising providing a processed CSF or blood sample obtained from a subject, wherein the CSF or blood sample is (a) depleted of N-terminal tau and mid-domain tau, and (b) enriched for MTBR tau; and quantifying, in the processed sample, (a) MTBR tau species comprising the amino sequence of SEQ ID NO: 9 (VQIVYKPVDLSK), and (b) MTBR tau species comprising the amino sequence of SEQ ID NO: 2 (IGSTENLK), SEQ ID NO: 4 (VQIINK), SEQ ID NO: 5 (LDLSNVQSK), SEQ ID NO: 6 (HVPGGGSVQIVYKPVDLSK), SEQ ID NO: 7 (IGSLDNITHVPGGGNK), SEQ ID NO: 8 (IGSLDNITHVPGGGN), or combinations thereof, wherein the ratio of quantified MTBR species from (a) and (b) discriminates a 3R-tauopathy from other tauopathies and a healthy state.

In another specific embodiment, the present disclosure provides a method for measuring tauopathy-related pathology in a subject, the method comprising providing a processed CSF or blood sample obtained from a subject, wherein the CSF or blood sample is depleted of mid-domain tau and enriched for MTBR tau; and quantifying, in the processed sample, MTBR species comprising the amino sequence of SEQ ID NO: 2 (IGSTENLK), MTBR species comprising the amino sequence of SEQ ID NO: 3 (LQTAPVPMPDLK), MTBR species comprising the amino sequence of SEQ ID NO: 4 (VQIINK), SEQ ID NO: 5 (LDLSNVQSK), MTBR species comprising the amino sequence of SEQ ID NO: 6 (HVPGGGSVQIVYKPVDLSK), SEQ ID NO: 8 (IGSLDNITHVPGGGN), MTBR species comprising the amino sequence of SEQ ID NO: 9 (VQIVYKPVDLSK), or a combination thereof, wherein the amount of the quantified MTRB-tau species, or their ratios, is a representation of tauopathy-related pathology in a brain of a subject.

In another specific embodiment, the present disclosure provides a method for measuring tau deposition in a brain in a subject, the method comprising providing a processed CSF or blood sample obtained from a subject, wherein the CSF or blood sample is depleted of mid-domain tau and enriched for MTBR tau; and quantifying, in the processed sample, MTBR species comprising the amino sequence of SEQ ID NO: 2 (IGSTENLK), MTBR species comprising the amino sequence of SEQ ID NO: 3 (LQTAPVPMPDLK), MTBR species comprising the amino sequence of SEQ ID NO: 4 (VQIINK), SEQ ID NO: 5 (LDLSNVQSK), MTBR species comprising the amino sequence of SEQ ID NO: 6 (HVPGGGSVQIVYKPVDLSK), SEQ ID NO: 8 (IGSLDNITHVPGGGN), MTBR species comprising the amino sequence of SEQ ID NO: 9 (VQIVYKPVDLSK), or a combination thereof, wherein the amount of the quantified MTRB-tau species, or their ratios, is a representation of tau deposition in a brain of a subject.

In another specific embodiment, the present disclosure provides a method for measuring tau deposition in a brain in a subject, the method comprising providing a processed CSF or blood sample obtained from a subject, wherein the CSF or blood sample is depleted of mid-domain tau and enriched for MTBR tau; and quantifying, in the processed sample, MTBR species comprising the amino sequence of SEQ ID NO: 2 (IGSTENLK), MTBR species comprising the amino sequence of SEQ ID NO: 4 (VQIINK), SEQ ID NO: 5 (LDLSNVQSK), MTBR species comprising the amino sequence of SEQ ID NO: 6 (HVPGGGSVQIVYKPVDLSK), SEQ ID NO: 8 (IGSLDNITHVPGGGN), MTBR species comprising the amino sequence of SEQ ID NO: 9 (VQIVYKPVDLSK), or a combination thereof, wherein the amount of the quantified MTRB-tau species, or their ratios, is a representation of tau deposition in a brain of a subject with a 3R-tauopathy or a 4R-tauopathy (i.e., non-AD tauopathy).

The specific embodiments that follow are directed to methods that comprise a method for measuring tau in a biological sample. In each of these embodiments, the method for measuring tau in a biological sample may comprise (a) decreasing in a biological sample by affinity depletion N-terminal tau, mid-domain tau, or N-terminal tau and mid-domain tau, and optionally decreasing by affinity depletion amyloid beta, wherein the biological sample is a blood sample or a CSF sample and the biological sample optionally comprises an isotope-labeled, tau internal standard; (b) enriching tau by a method that comprises (i) removing additional proteins from the biological sample by protein precipitation and separation of the precipitated proteins to obtain a supernatant, and then purifying tau from the supernatant by solid phase extraction, or (ii) affinity purifying MTBR tau, thereby producing by either (i) or (ii) enriched tau; (c) cleaving the enriched tau with a protease and then optionally desalting the resultant cleavage product by solid phase extraction to obtain a sample comprising proteolytic peptides of tau; and (d) performing liquid chromatography-mass spectrometry (LC/MS) of the sample comprising proteolytic peptides of tau to detect and measure the amount of at least one proteolytic peptide of tau. Further details for each of steps (a) to (d) can be found in Section II, incorporated herein by reference.

In another specific embodiment, the present disclosure provides a method for measuring Alzheimer disease (AD)-related pathology in a subject, the method comprising measuring tau in a biological sample according to the above-referenced method, wherein the tau measured are MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 8 (IGSLDNITHVPGGGN), or a combination thereof, wherein the amount of the MTRB-tau species is a representation of AD-related pathology in a brain of a subject.

In another specific embodiment, the present disclosure provides a method for measuring Alzheimer disease (AD)-related tau deposition in a brain of a subject, the method comprising measuring tau in a biological sample according to the above-referenced method, wherein the tau measured are MTBR tau species comprising the amino sequence SEQ ID NO: 3 (LQTAPVPMPDLK), and wherein the amount of the MTRB-tau species is a representation of AD-related pathology in a brain of a subject.

In another specific embodiment, the present disclosure provides a method for measuring Alzheimer disease (AD)-related tau deposition in a brain of a subject, the method comprising measuring tau in a biological sample according to the above-referenced method, wherein the tau measured are MTBR tau species comprising the amino sequence of SEQ ID NO: 3 (LQTAPVPMPDLK), MTBR tau species comprising the amino sequence OF SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof, and wherein the amount of the MTRB-tau species is a representation of AD-related pathology in a brain of a subject.

In another specific embodiment, the present disclosure provides a method for determining a subject's amyloid status, the method comprising measuring tau in a biological sample according to the above-referenced method, wherein the tau measured are MTBR tau species comprising the amino sequence of SEQ ID NO: 6 (HVPGGGSVQIVYKPVDLSK), and wherein the amount of the MTRB-tau species is a representation of AD-related amyloid beta deposition in a brain of a subject and predicts amyloid-positivity as determined by PIB-PET.

In another specific embodiment, the present disclosure provides a method for diagnosing Alzheimer's disease, the method comprising measuring tau in a biological sample according to the above-referenced method, wherein the tau measured are MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof; and diagnosing Alzheimer's disease when the quantified MTBR tau species differs by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF.

In another specific embodiment, the present disclosure provides a method for measuring Alzheimer disease (AD) progression in a subject, the method comprising measuring tau in a biological sample according to the above-referenced method, wherein the tau measured are MTBR tau species comprising the amino sequence of ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or a combination thereof; and calculating the difference between the quantified MTBR tau species in the second sample and the first sample, wherein a statistically significant increase in the quantified MTBR tau species in the second sample indicates progression of the subject's Alzheimer's disease.

In another specific embodiment, the present disclosure provides a method for discriminating a 4R-tauopathy, the method comprising measuring tau in a biological sample according to the above-referenced method, wherein the tau measured are (i) MTBR tau species comprising the amino sequence of SEQ ID NO: 9 (VQIVYKPVDLSK), and (ii) MTBR tau species comprising the amino sequence of SEQ ID NO: 7 (IGSLDNITHVPGGGNK) or MTBR tau species comprising the amino sequence of SEQ ID NO: 8 (IGSLDNITHVPGGGN); wherein the ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising measuring tau in a biological sample according to the above-referenced method, wherein the tau measured are (i) MTBR tau species comprising the amino sequence of SEQ ID NO: 9 (VQIVYKPVDLSK), and (ii) MTBR tau species comprising the amino sequence of SEQ ID NO: 4 (VQIINK), MTBR tau species comprising the amino sequence of SEQ ID NO: 5 (LDLSNVQSK), MTBR tau species comprising the amino acid sequence of SEQ ID NO: 6 (HVPGGGSVQIVYKPVDLSK), or any combination thereof; wherein a ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising measuring tau in a biological sample according to a method of any one of claims 6 to 17, wherein the tau measured are (i) MTBR tau species comprising the amino sequence of SEQ ID NO: 2 (IGSTENLK), MTBR tau species comprising the amino sequence of SEQ ID NO: 4 (VQIINK), MTBR tau species comprising the amino sequence of SEQ ID NO: 5 (LDLSNVQSK), or combinations thereof, and (ii) MTBR tau species comprising the amino acid sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising measuring tau in a biological sample according to the above-referenced method, wherein the tau measured are (i) MTBR tau species comprising the amino sequence of SEQ ID NO: 2 (IGSTENLK), MTBR tau species comprising the amino sequence of SEQ ID NO: 4 (VQIINK), MTBR tau species comprising the amino sequence of SEQ ID NO: 5 (LDLSNVQSK), or combinations thereof, and (ii) MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (i) and (ii) discriminates a 4R-tauopathy from Alzheimer's disease and a healthy state.

In another specific embodiment, the present disclosure provides a method for discriminating a 4R-tauopathy, the method comprising measuring tau in a biological sample according to the above-referenced method, wherein the tau measured are (i) MTBR tau species comprising the amino sequence of SEQ ID NO: 6 (HVPGGGSVQIVYKPVDLSK), and (ii) MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof, wherein the ratio of quantified MTBR species from (i) and (ii) discriminates a 4R-tauopathy from Alzheimer's disease and a healthy state.

In another specific embodiment, the present disclosure provides a method for measuring tauopathy-related pathology in a subject, the method comprising measuring tau in a biological sample according to the above-referenced method, wherein the CSF or blood sample is depleted of mid-domain tau and enriched for MTBR tau; and quantifying, in the processed sample, MTBR species comprising the amino sequence of SEQ ID NO: 2 (IGSTENLK), MTBR species comprising the amino sequence of SEQ ID NO: 3 (LQTAPVPMPDLK), MTBR species comprising the amino sequence of SEQ ID NO: 4 (VQIINK), SEQ ID NO: 5 (LDLSNVQSK), MTBR species comprising the amino sequence of SEQ ID NO: 6 (HVPGGGSVQIVYKPVDLSK), SEQ ID NO: 8 (IGSLDNITHVPGGGN), MTBR species comprising the amino sequence of SEQ ID NO: 9 (VQIVYKPVDLSK), or a combination thereof, wherein the amount of the quantified MTRB-tau species, or their ratios, is a representation of tauopathy-related pathology in a brain of a subject.

In a specific embodiment, the present disclosure provides a method for measuring tau deposition in a brain in a subject, the method comprising measuring tau in a biological sample according to the above-referenced method, wherein the CSF or blood sample is depleted of mid-domain tau and enriched for MTBR tau; and quantifying, in the processed sample, MTBR species comprising the amino sequence of SEQ ID NO: 2 (IGSTENLK), MTBR species comprising the amino sequence of SEQ ID NO: 3 (LQTAPVPMPDLK), MTBR species comprising the amino sequence of SEQ ID NO: 4 (VQIINK), SEQ ID NO: 5 (LDLSNVQSK), MTBR species comprising the amino sequence of SEQ ID NO: 6 (HVPGGGSVQIVYKPVDLSK), SEQ ID NO: 8 (IGSLDNITHVPGGGN), MTBR species comprising the amino sequence of SEQ ID NO: 9 (VQIVYKPVDLSK), or a combination thereof, wherein the amount of the quantified MTRB-tau species, or their ratios, is a representation of tau deposition in a brain of a subject.

In another specific embodiment, the present disclosure provides a method for treating a subject in need thereof, the method comprising (a) providing a processed CSF or blood sample obtained from a subject, wherein the CSF or blood sample is (i) depleted of mid-domain tau, and (ii) enriched for MTBR tau; (b) quantifying, in the processed sample, MTBR tau species comprising the amino acid sequence of SEQ ID NO: 2 (IGSTENLK), MTBR tau species comprising the amino sequence of SEQ ID NO: 3 (LQTAPVPMPDLK), MTBR tau species comprising the amino sequence of SEQ ID NO: 4 (VQIINK), MTBR tau species comprising the amino sequence of SEQ ID NO: 5 (LDLSNVQSK), MTBR tau species comprising the amino acid sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), MTBR tau species comprising the amino sequence of SEQ ID NO: 9 (VQIVYKPVDLSK), or combinations thereof; and (c) administering a treatment to the subject to alter tau pathology, wherein the subject's processed CSF or blood sample has quantified MTBR tau species, or ratios of the quantified MTBR tau species, that differ by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the quantified MTRB-tau species or their ratios is a representation of tau pathology in a brain of a subject. In some embodiments, administering a treatment to the subject to alter tau pathology alters or stabilizes the amount of the quantified MTBR species. In some embodiments the treatment is a pharmaceutical composition comprising a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen-binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-AP antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor.

In another specific embodiment, the present disclosure provides a method for treating a subject in need thereof, the method comprising (a) providing a processed CSF or blood sample obtained from a subject, wherein the CSF or blood sample is (i) depleted of mid-domain tau, and (ii) enriched for MTBR tau; (b) quantifying, in the processed sample, MTBR tau species comprising the amino sequence of SEQ ID NO: 3 (LQTAPVPMPDLK), MTBR tau species comprising the amino sequence of SEQ ID NO: 6 (HVPGGGSVQIVYKPVDLSK), MTBR tau species comprising the amino sequence of SEQ ID NO: 7 (IGSLDNITHVPGGGNK), MTBR tau species comprising the amino sequence of SEQ ID NO: 8 (IGSLDNITHVPGGGN), or combinations thereof; and (c) administering a treatment to the subject to alter tau pathology, wherein the subject's processed CSF or blood sample has quantified MTBR tau species, or ratios of the quantified MTBR tau species, that differ by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the quantified MTRB-tau species or their ratios is a representation of tau pathology in a brain of a subject. In some embodiments, administering a treatment to the subject to alter tau pathology alters or stabilizes the amount of the quantified MTBR species. In some embodiments the treatment is a pharmaceutical composition comprising a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen-binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-AP antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1

Several sample processing methods were developed—an immunoprecipitation method for N-terminal tau and mid-domain tau (IP), described in Sato et al., 2018; a chemical extraction method (CX); and a process combining the IP and CX methods to enrich for MTBR tau (PostIP-CX). The CX and PostIP-CX methods were specifically developed to detect and quantify MTBR tau. An overview of these methods is provided in FIG. 2.

Briefly, CSF (about 475 μL) was mixed with a solution containing ¹⁵N Tau-441(2N4R) Uniform Labeled (approximately 10 μL of 100 pg/μL solution, or approximately 5 μL of a 200 pg/μL solution) as an internal standard. N-terminal tau and mid-domain tau species were immunoprecipitated with Tau1 and HJ8.5 antibodies, and then processed and trypsin digested as described previously (Sato et al., 2018).

For the CX method, CSF (about 475 μL) was mixed with a solution containing ¹⁵N Tau-441(2N4R) Uniform Labeled (approximately 10 μL of 100 pg/μL solution, or approximately 5 μL of a 200 pg/μL solution) as an internal standard. Then, tau was chemically extracted. Highly abundant CSF proteins were precipitated using 25 μL of perchloric acid. After mixing and incubation on ice for 15 minutes, the mixture was centrifuged at 20,000 g for 15 minutes at 4° C., and the supernatant was further purified using the Oasis HLB 96-well μElution Plate (Waters) according to the following steps. The plate was washed once with 300 μL of methanol and equilibrated once with 500 μL of 0.1% FA in water. The supernatant was added to the Oasis HLB 96-well μElution Plate and adsorbed to the solid phase. Then, the solid phase was washed once with 500 μL of 0.1% FA in water. Elution buffer (100 μL; 35% acetonitrile and 0.1% FA in water) was added, and the eluent was dried by Speed-vac. Dried sample was dissolved by 50 μL of trypsin solution (10 ng/μL) in 50 mM TEABC and incubated at 37° C. for 20 hours.

For the PostIP-CX method, the post-immunoprecipitated CSF (i.e., the supernatant remaining after the IP method described above) was processed as described in the CX method.

Following tryptic digestion, all samples were purified by solid phase extraction on C18 TopTip. In this purification process, 5 fmol each of AQUA internal-standard peptide for residues 354-369 (MTBR tau-354) and 354-368 (tau368) was spiked for the differential quantification. Before eluting samples, 3% hydrogen peroxide and 3% FA in water were added to the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system coupled to Orbitrap Fusion Lumos Tribrid or Orbitrap Tribrid Eclipse mass spectrometer (Thermo Scientific) operating in PRM mode.

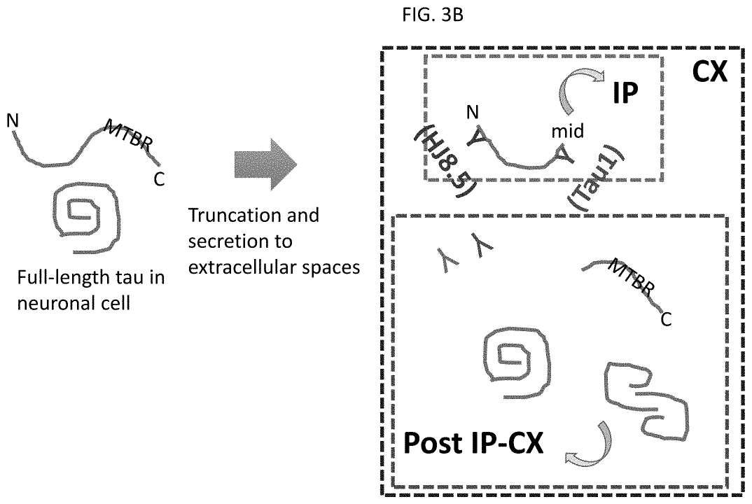

As shown in FIG. 3A, the CX and PostIP-CX methods produced samples comprising MTBR tau detectable and quantifiable by mass spectrometry. Quantifiable signals of MTBR tau were not obtained by the IP method. Although not demonstrated, it is believed alternative methods for detecting and quantifying MTBR tau that have similar sensitivity may also be used.

### Example 2

In this example, CSF samples from two clinical cohorts of subjects with late onset Alzheimer's disease (LOAD100 and LOAD60) were analyzed. Clinical dementia rating (CDR) scores and amyloid status for the samples used in this analysis are provided in Tables 1 and 2. CSF samples (about 500 μl each) were processed by the PostIP-CX method and evaluated by mass spectrometry, as described in Example 1. CSF Aβ42 and Aβ40 immunoprecipitated from the CSF was measured by mass spectrometry as described previously (Patterson B W, et al., Ann Neurol 2015, 78: 439-453). pT217% was measured by mass spectrometry as described previously (Barthélemy, N. R., et al., Alz Res Therapy, 2020, 12: 26).

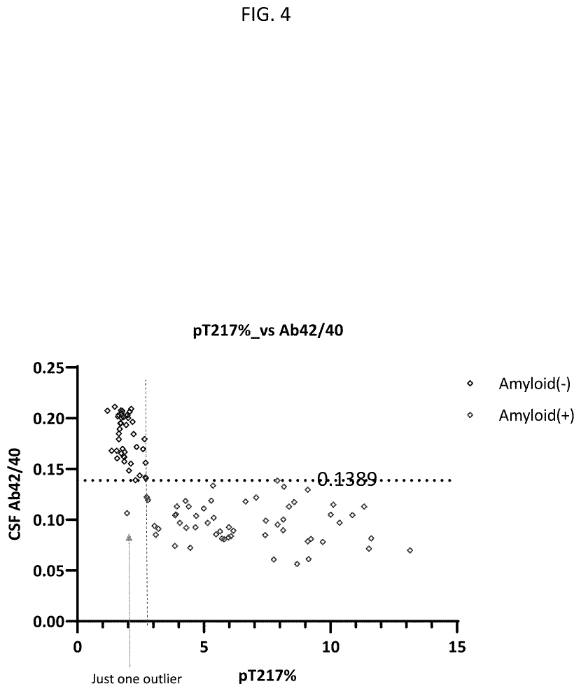

A cutoff value for CSF Aβ42/40 was calculated from PiB-PET SUVR results to determine amyloid status. Based on the established cutoff >1.42 for PiB-PET SUVR (Ann Neurol 2016; 80:379-387), (Sensitivity %+Specificity %) was maximized at 0.1389 for CSF Aβ42/40. Notably, pT217% showed excellent correlation with amyloid status defined by the established cutoff, with only a single outlier (FIG. 4).

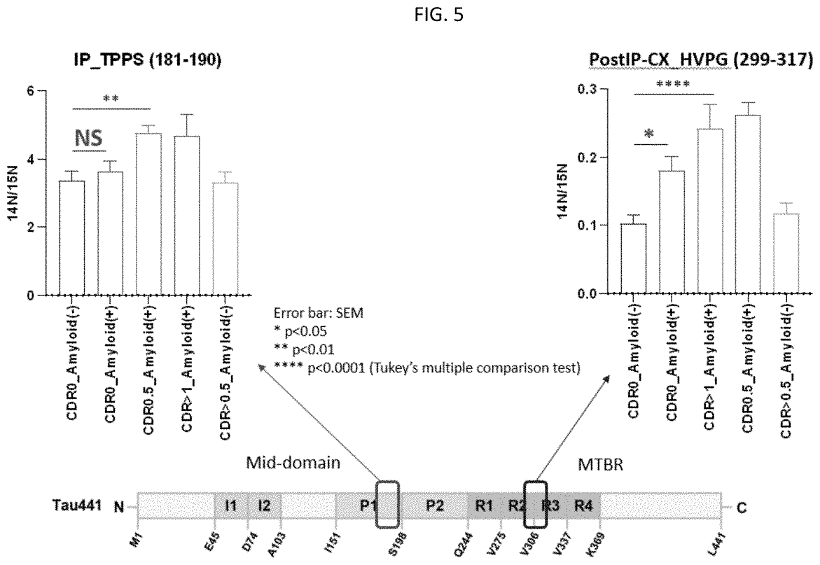

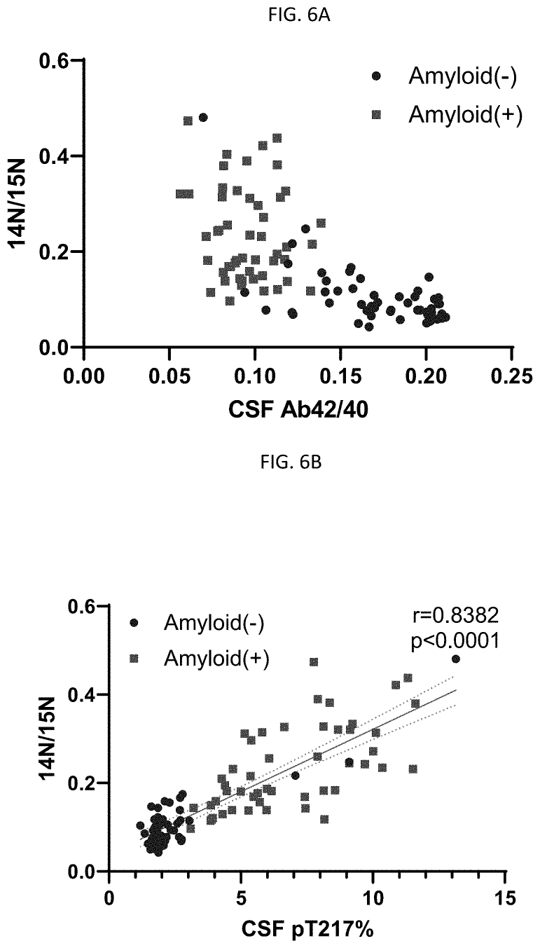

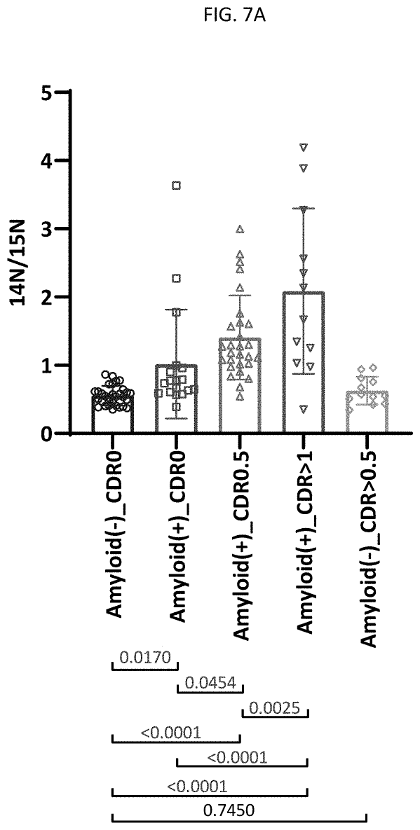

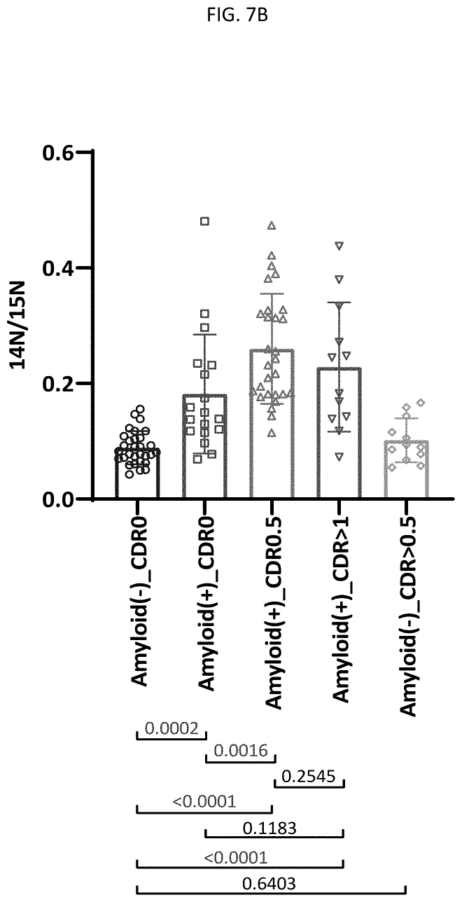

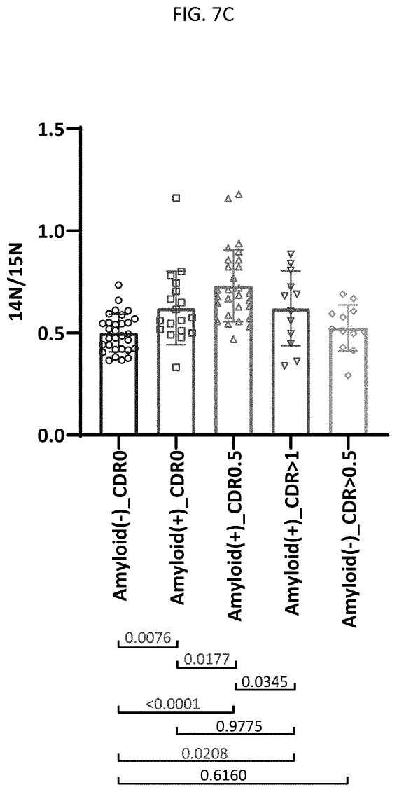

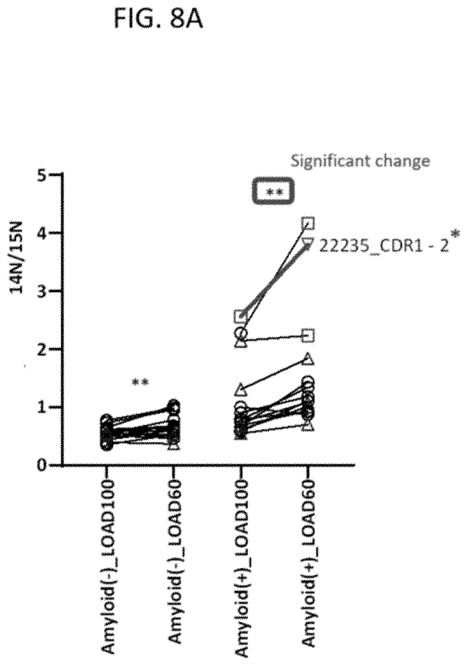

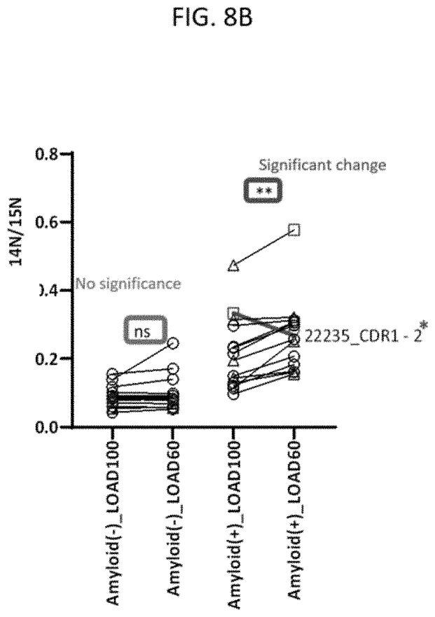

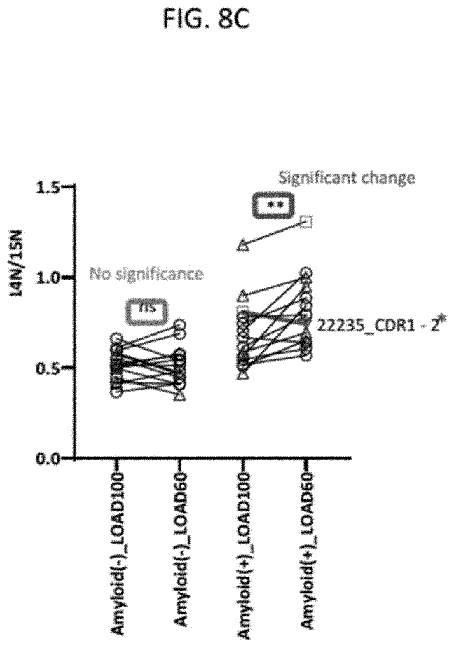

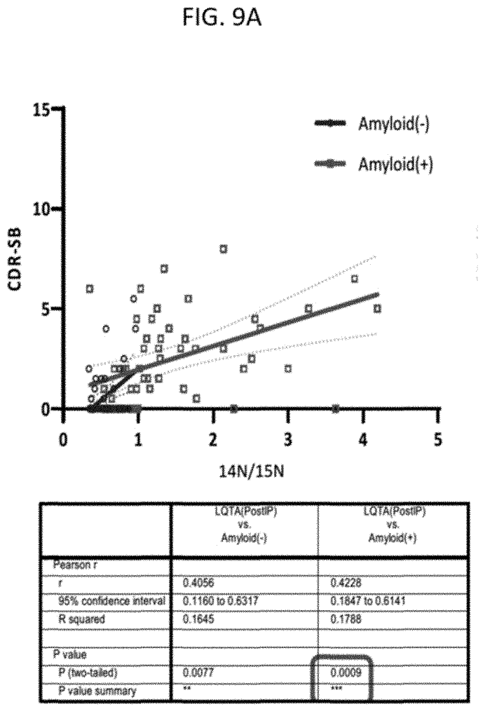

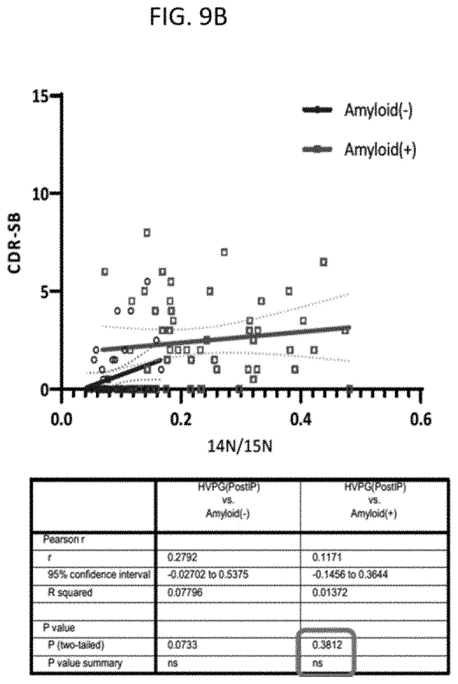

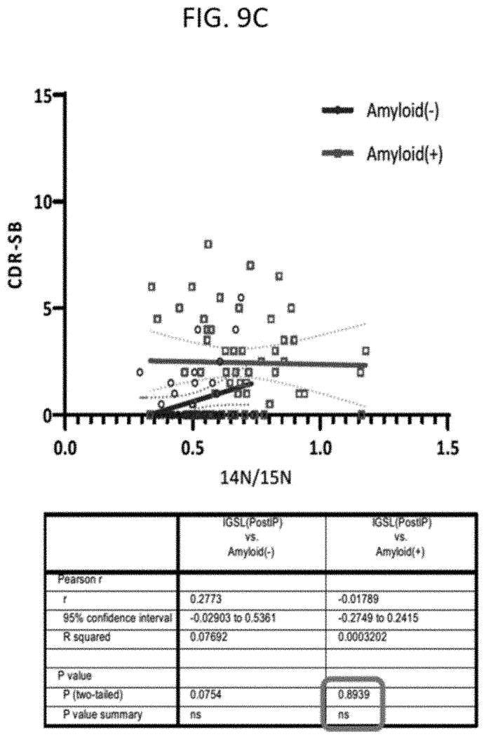

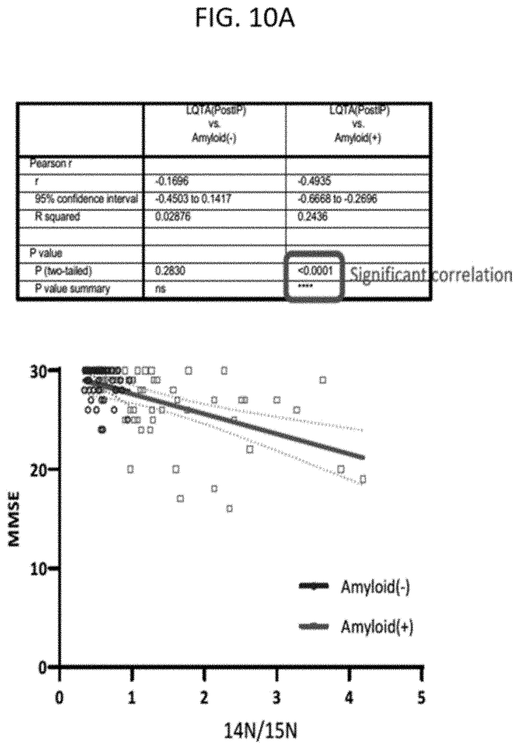

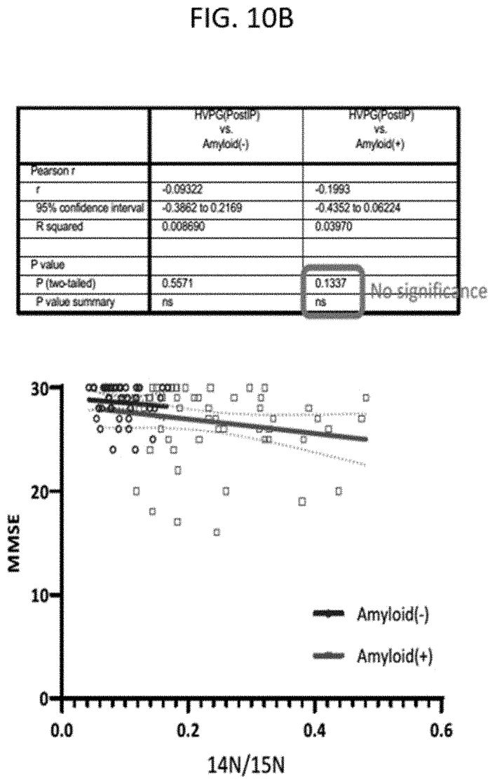

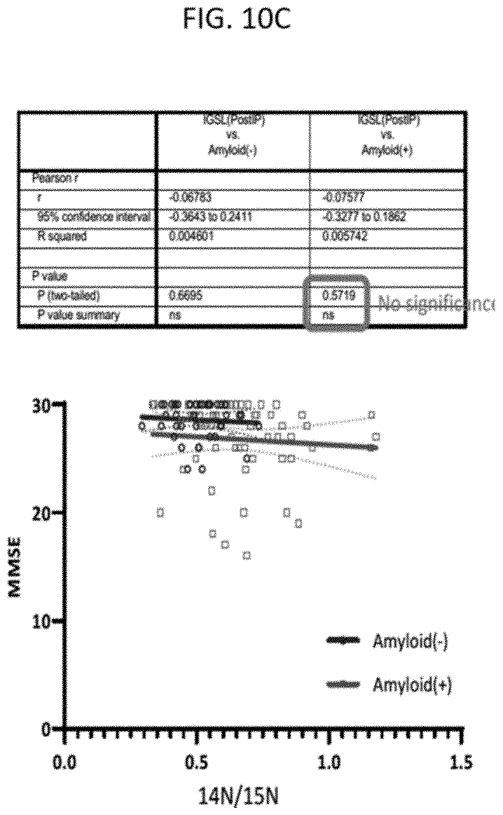

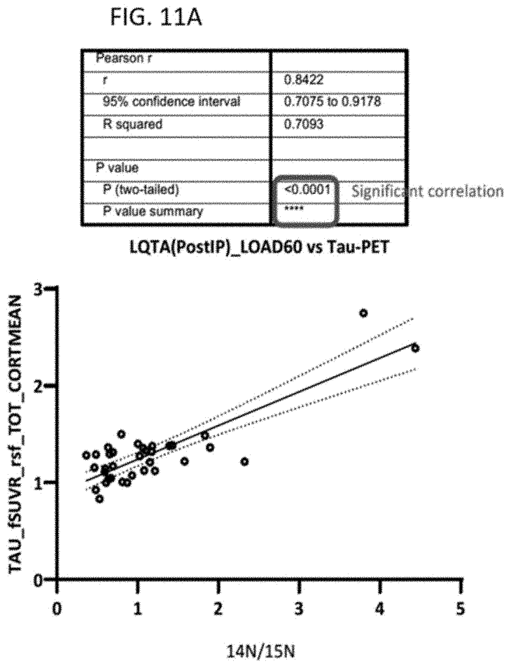

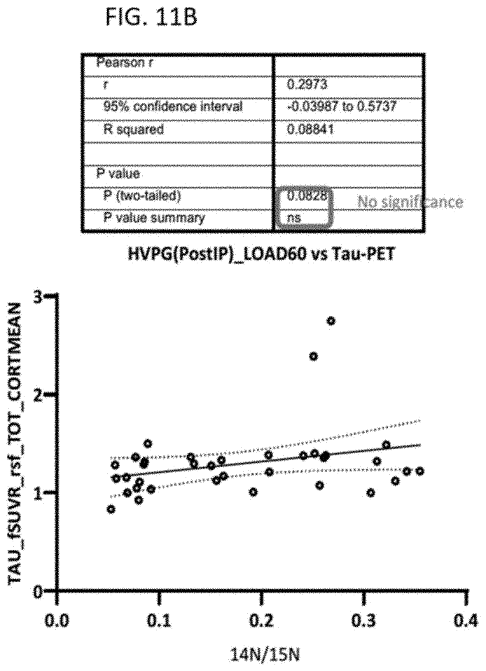

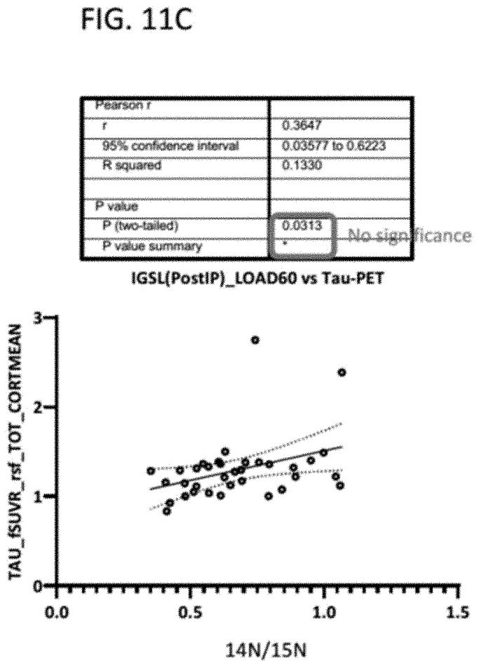

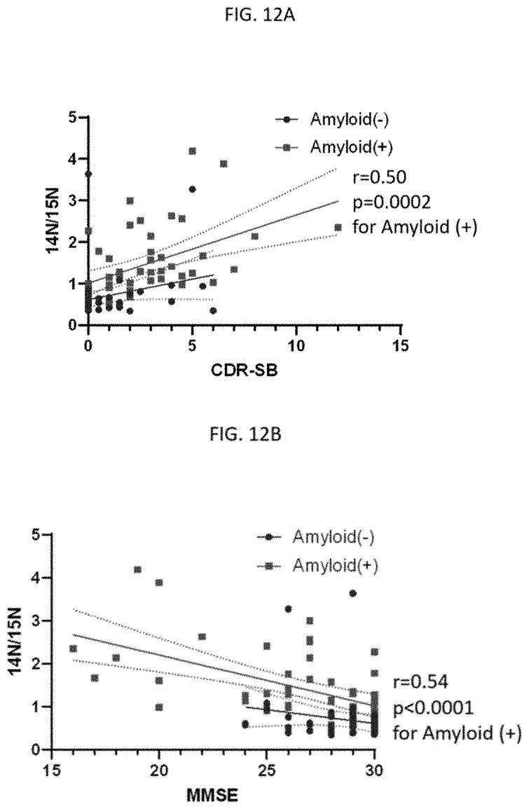

As shown in FIG. 5-7, tryptic peptides of tau associated with the MTBR, measured in PostIP-CX samples, were specifically increased in amyloid positive subjects. HVPG was the most significantly increased, even in clinically asymptomatic stages (FIG. 5-6). The increases of HVPG and IGSL were saturated after symptomatic onset, whereas LQTA continued to increase even after the clinical onset (FIG. 7-8). The concentration of LQTA showed the highest correlation with tau pathology as measured by positron emission tomography (PET) for tau (Pearson r=0.84, n=35), and also with cognitive testing measures (FIG. 9-12). For some of the above analyses, data for amyloid positive CDR 1 and CDR 2 samples were combined as CDR>1, and data for amyloid negative CDR 0.5 and CDR 1 were combined as CDR>0.5. Statistical analyses were conducted by one-way ANOVA adjusted for multiple comparisons using Benjamini-Hochberg FDR method with FDR set at 5%.

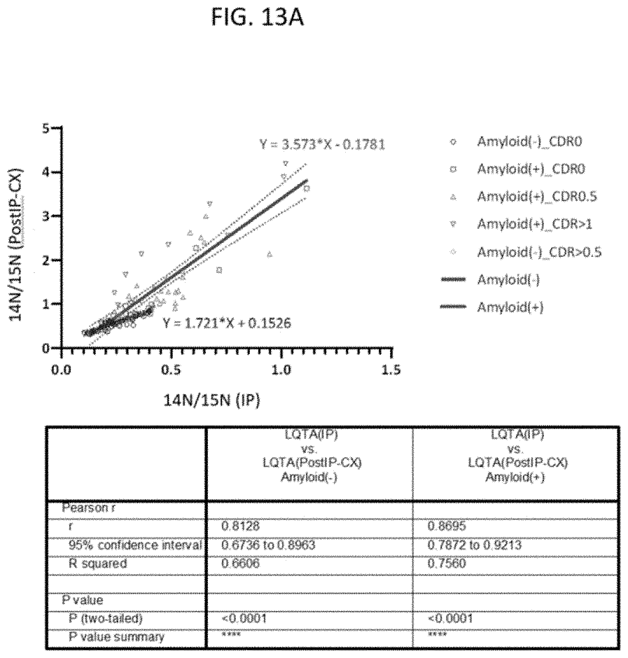

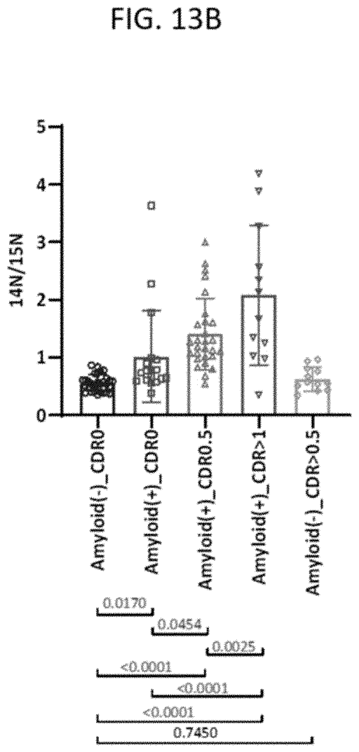

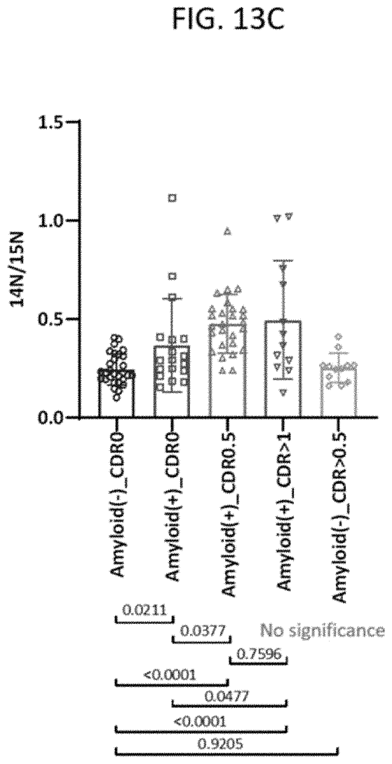

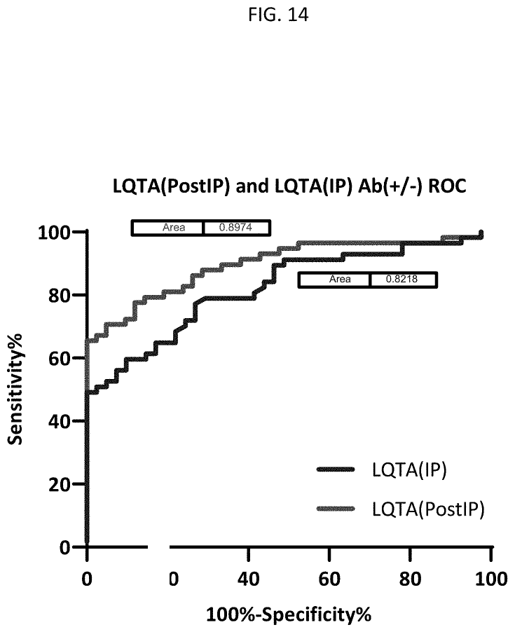

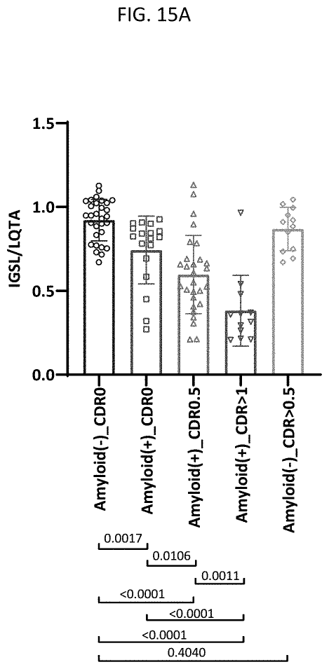

Importantly, sample processing was shown to affect the diagnostic utility of tau. As an example, whereas the tryptic peptide HVPG of MTBR tau differentiates amyloid positive from amyloid negative subjects in the preclinical stages in PostIP-CX samples, this discriminatory power was not observed with the tryptic peptide TPPS of mid-domain tau in IP samples (FIG. 5). The amino acid sequence of the TPPS tryptic peptide is TPPSSGEPPK (SEQ ID NO: 10). Sample processing was also shown to significantly influence the ability to discriminate changes in the amount of MTBR tau between the various CSF samples. For example, the tryptic peptide LQTA shows a linear increase in CSF samples after the symptomatic stage in PostIP-CX samples but not in IP samples (FIG. 13), indicating PostIP-LQTA (mid-domain-independent MTBR tau-243) clearly discriminates amyloid status better than IP-LQTA (FIG. 14).

The above data suggest MTBR tau, which are enriched in AD brain aggregates, are also increased in AD CSF. It is hypothesized that the tryptic peptides LQTA and HVPG are part of the fuzzy-coat and starting point of tau filament aggregation, respectively, whereas IGSL is inside the core. The position of the LQTA peptide on the surface of filament as fuzzy coat may always expose it, increasing the likelihood of its release into CSF. Given the role of HVPG in aggregation, immature filament may still be exposing HPVG on the surface while the peptide may be recruited in the core of mature filament. IGSL's posited position in the core of filament may be expected at even early AD stages.

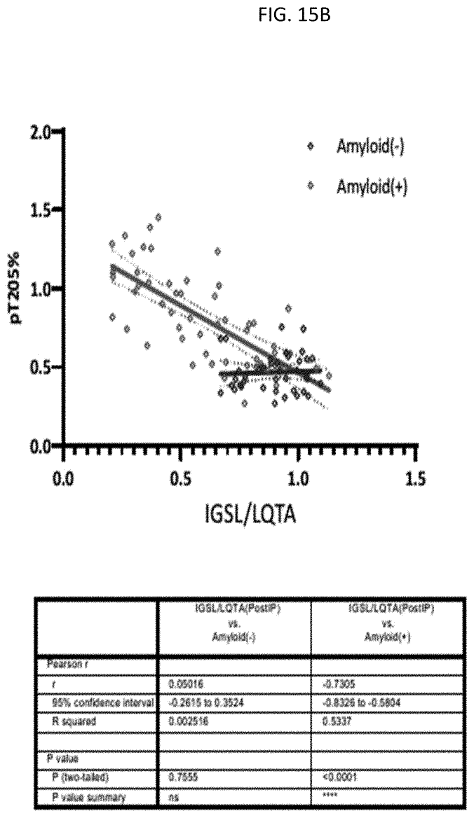

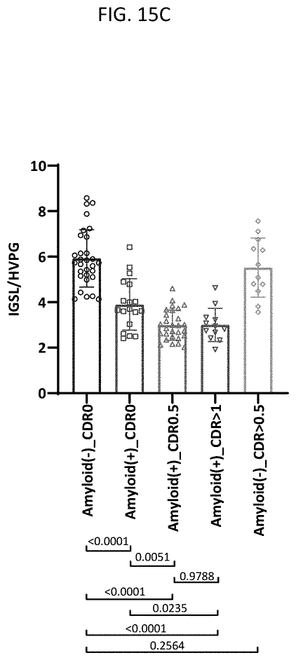

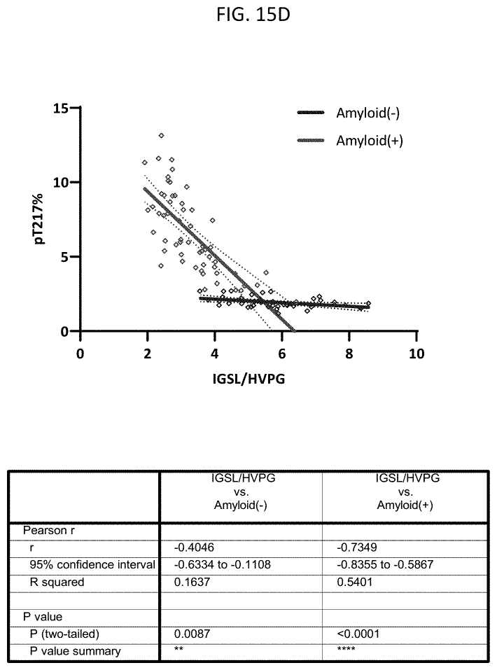

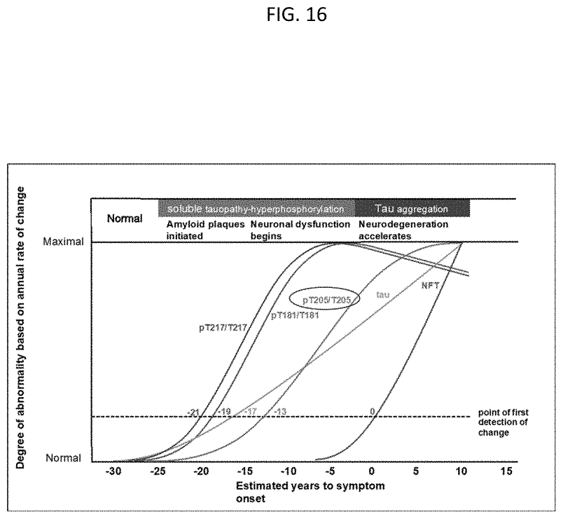

Regardless of the underlying mechanism, the data suggest the tryptic peptides HVPG and LQTA in CSF may be used as biomarkers to recapitulate amyloid status and tau pathology in AD, respectively. Notably, only LQTA showed the continuous increase along disease progression in terms of tau-PET, as well as amyloid status and cognitive decline, which suggests the region is key to differentiating tau pathology in AD. The use of these peptides in combination with the tryptic peptide IGSL and/or with other biomarkers will boost the discrimination power when staging a subject's disease trajectory (FIG. 15-17). In addition, LQTA and other MTBR tau peptides may be used as biomarkers to differentiate various tauopathies.

TABLE 1
LOAD100 demographics

| | | |
|---|---|---|
| Amyloid (−) | CDR 0 | n = 35 |
| CSF Aβ42/40 > 0.1389 | CDR 0.5 | n = 12 |
| | CDR 1 | n = 3 |
| Amyloid (+) | CDR 0 | n = 13 |
| CSF Aβ42/40 < 0.1389 | CDR 0.5 | n = 27 |
| | CDR 1 | n = 9 |
| | CDR 2 | n = 1 |

TABLE 2
LOAD60 demographics

| | | |
|---|---|---|
| Amyloid (−) | CDR 0 | n = 13 |
| | CDR 0.5 | n = 3 |
| | CDR 1 | n = 3 |
| Amyloid (+) | CDR 0 | n = 7 |
| | CDR 0.5 | n = 3 |
| | CDR 1 | n = 2 |
| | CDR 2 | n = 1 |
| Amyloid undefined | | n = 27 |

### Example 3

In this example, the presence and potential utility of MTBR tau species as Alzheimer's disease biomarkers is described in detail. The results show that a significant amount of mid-domain-independent MTBR tau exists in CSF—i.e., tau species that have been cleaved near the center of the polypeptide sequence (e.g., around amino acid 224 of tau-441) resulting in a C-terminal fragment (a “C-terminal stub”) that lacks the N-terminus and the mid-domain regions. Moreover, different regions of CSF MTBR tau stage disease progression and correlate with tau aggregation within the Alzheimer's disease brain. These findings provide new insights into the relationship between MTBR tau in the brain and CSF and support the use of CSF tau as a fluid biomarker for Alzheimer's disease.

Materials: Two different cohorts of human brain samples were used in the experiments of this example—a discovery cohort and a validation cohort. The discovery cohort contained postmortem frozen brain tissue samples from two participants with Alzheimer's disease pathology and two control participants without pathology, which were provided by the Knight ADRC Pathology Core at Washington University School of Medicine. Each sample was classified according to the National Institute on Aging and Alzheimer's Association amyloid stage A3 (ThaI phase) for amyloid deposition and Tau Braak stage VI, B3 for tau aggregation. Samples from each participant were collected from six to ten brain regions including the cerebellum, superior frontal gyrus, frontal pole, temporal, occipital, thalamus, amygdala, pons, parietal and striatum. Additional postmortem frozen brain tissue samples from the parietal lobe were analyzed from 20 participants (eight amyloid-negative and 12 amyloid-positive by CSF Aβ 42/40 ratios) as a validation cohort. The 12 amyloid-positive samples were further divided into clinical groups according to their Clinical Dementia Rating (CDR) scores, and classified as very mild to moderate Alzheimer's disease (amyloid-positive, CDR=0.5−2, n=5) or severe Alzheimer's disease (amyloid-positive, CDR=3, n=7). These human studies were approved by the Washington University Institutional Review Board.

Three different cohorts of human CSF samples were also used in the experiments of this example—a cross-sectional cohort, a longitudinal cohort, and a Tau PET cohort (Table 3A and Table 3B). CSF samples from 100 participants were collected from the amyloid beta (Aβ) stable isotope labeling kinetics (SILK) study (Patterson et al., 2015) for analysis as a cross-sectional cohort. This cohort is also referred to as the LOAD100 cohort in Example 2. CSF collection was performed as previously described (Patterson et al., 2015). Briefly, CSF was collected at baseline. Next, participants received a leucine bolus infusion over 10 minutes. Six mL of CSF was obtained hourly for 36 hours. CSF aliquots collected at hour 30 were used for MS measurement of tau species in this study. Amyloid status was defined using CSF Aβ 42/40 ratio as previously reported (Patterson et al., 2015). The corresponding cutoff ratio (0.1389) maximized the accuracy in predicting amyloid-positivity as determined by Pittsburgh compound B (PiB) PET. Amyloid groups were further divided into clinical groups according to their CDR scores as shown in Table 3A. From the cross-sectional cohort, 28 participants (14 amyloid-positive and 14 amyloid-negative) were followed for two to nine years to assess the longitudinal trajectory of tau species in CSF. CSF samples were collected and analyzed in the same manner as the cross-sectional cohort. The Tau PET cohort contained thirty-five participants (20 amyloid-positive and 15 amyloid-negative, including 16 participants from longitudinal cohort) who had tau PET AV-1451 standardized uptake value ratio (SUVR) measures within three years from the time of CSF collection. PET scans were performed as previously described (Sato et al., 2018) and the partial-volume correction was performed for SUVR using a regional spread function technique (Su et al., 2015). CSF samples were collected and analyzed in the same manner as the other cohorts.

TABLE 3A
Cross-sectional cohort

| | Cross-sectional cohort (n = 100) | | | | | | | | | |
|---|---|---|---|---|---|---|---|---|---|---|
| Variable | Control | | Preclinical AD | | Very Mild AD | | Mild-Moderate AD | | Non-AD CI | |
| n | 30 | | 18 | | 28 | | 12 | | 12 | |
| Age | 71 | (5) | 73 | (7) | 75 | (7) | 72 | (8) | 75 | (8) |
| Gender (F/M) | 18/12 | | 11/7 | | 11/17 | | 2/10 | | 2/10 | |
| CDR | 0 | | 0 | | 0.5 | | 1-2 | (>1) | 0.5-1 | (>0.5) |
| CSF Aβ 42/40 | 0.18 | (0.02) | 0.10 | (0.02) | 0.09 | (0.02) | 0.10 | (0.02) | 0.17 | (0.02) |
| PiB SUVR | 1.04 (0.11) 27 | | 1.99 (0.87) 16 | | 3.14 (0.92) 13 | | 2.73 (2.10) 2 | | 0.98 (0.05) 5 | |
| AV45 SUVR | 1.12 (0.42) 16 | | 1.85 (0.52) 8 | | 2.18 (0.53) 6 | | 2.41 (na) 1 | | 0.96 (0.15) 2 | |
| Amyloid status | negative | | positive | | positive | | positive | | negative | |
| AV-1451 SUVR | na | | na | | na | | na | | na | |
| CSF tau level (ng/mL) | | | | | | | | | | |
| MTBR tau-243 | 2.44 | (0.63) | 4.47 | (3.51) | 6.18 | (2.71) | 9.17 | (5.32) | 2.75 | (0.89) |
| MTBR tau-299 | 0.39 | (0.13) | 0.80 | (0.45) | 1.14 | (0.42) | 1.00 | (0.49) | 0.45 | (0.17) |
| MTBR tau-354 | 2.20 | (0.41) | 2.73 | (0.79) | 3.21 | (0.77) | 2.73 | (0.80) | 2.31 | (0.49) |

Data are shown as mean (SD). AD: Alzheimer's disease. CI: cognitive impairment. CDR: Clinical Dementia Rating. PiB: Pittsburgh compound B. AV-45: florbetapir. AV-1451: flortaucipir. SUVR: standardized uptake value ratio. na: not available. Superscript numbers indicate the number of available measures within the group. Amyloid statuses in longitudinal and tau PET cohorts were determined historically from the results of the cross-sectional cohort and amyloid PET, respectively. The concentration of each MTBR tau isoform (MTBR tau-243, MTBR tau-299, and MTBR tau-354) was determined by mass spectrometry following to chemical extraction method in post-immunoprecipitated CSF samples.

TABLE 3B
Longitudinal and Tau PET cohorts

| | Longitudinal cohort (n = 28) | | | | Tau PET cohort (n = 35) | | | |
|---|---|---|---|---|---|---|---|---|
| Variable | Control | | AD | | Control | | AD | |
| n | 14 | | 14 | | 15 | | 20 | |
| Age | 74 | (5) | 77 | (6) | 75 | (6) | 75 | (6) |
| Gender (F/M) | 5/9 | | 7/7 | | 12/3 | | 11/9 | |
| CDR | 0-0.5 | | 0-2 | | 0-0.5 | | 0-2 | |
| CSF Aβ 42/40 | na | | na | | na | | na | |
| PiB SUVR | 1.11 (0.18) 13 | | 2.47 (0.59) 11 | | 1.10 (0.13) 10 | | 2.37 (0.34) 14 | |
| AV45 SUVR | 0.98 (0.19) 9 | | 2.10 (0.46) 12 | | 0.98 (0.26) 13 | | 2.09 (0.49) 18 | |
| Amyloid status | negative | | positive | | negative | | positive | |
| AV-1451 SUVR | na | | na | | 1.23 | (0.14) | 1.75 | (0.69) |
| CSF tau level (ng/mL) | | | | | | | | |
| MTBR tau-243 | 2.94 | (0.87) | 7.09 | (4.76) | 2.70 | (0.67) | 6.82 | (4.26) |
| MTBR tau-299 | 0.44 | (0.23) | 1.18 | (0.47) | 0.40 | (0.21) | 1.07 | (0.31) |
| MTBR tau-354 | 2.24 | (0.48) | 3.61 | (0.89) | 2.33 | (0.64) | 3.43 | (0.72) |

Data are shown as mean (SD). AD: Alzheimer's disease. CI: cognitive impairment. CDR: Clinical Dementia Rating. PiB: Pittsburgh compound B. AV-45: florbetapir. AV-1451: flortaucipir. SUVR: standardized uptake value ratio. na: not available. Superscript numbers indicate the number of available measures within the group. Amyloid statuses in longitudinal and tau PET cohorts were determined historically from the results of the cross-sectional cohort and amyloid PET, respectively. The concentration of each MTBR tau isoform (MTBR tau-243, MTBR tau-299, and MTBR tau-354) was determined by mass spectrometry following to chemical extraction method in post-immunoprecipitated CSF samples.

Brain tau analysis by MS: Frozen brain tissue samples were sliced using a cryostat at −20° C. and collected in tubes. The tissue (300-400 mg) was sonicated in ice-cold buffer containing 25 mM tris-hydrochloride (pH 7.4), 150 mM sodium chloride, 10 mM ethylenediaminetetraacetic acid, 10 mM ethylene glycol tetraacetic acid, phosphatase inhibitor cocktail, and protease inhibitor cocktail at a concentration of 0.3 mg/μL of brain tissue. The homogenate was clarified by centrifugation for 20 minutes at 11,000 g at 4° C. The supernatant (whole brain extract) was aliquoted into new tubes and kept at −80° C. until use. The whole brain extract was incubated with 1% sarkosyl for 60 minutes on ice, followed by ultra-centrifugation at 100,000 g at 4° C. for 60 minutes to obtain an insoluble pellet. The insoluble pellet was resuspended with 200 μL of PBS followed by sonication and the insoluble suspension was kept at −80° C. until use.

For soluble tau analysis, tau species in whole brain extract were immunoprecipitated with Tau1 and HJ8.5 antibodies. Immunoprecipitated soluble tau species were processed and digested as described previously (Sato et al., 2018).

For insoluble tau analysis, insoluble suspension (10 to 20 μL containing 2.5 μg of total protein) was mixed with 200 μL of lysis buffer (7 M urea, 2 M thio-urea, 3% 3-[(3-cholamidopropyl)dimethylammonio]-1-propanesulfonate, 1.5% n-octyl glucoside, 100 mM triethyl ammonium bicarbonate (TEABC)) followed by spiking with 5 μL of solution containing ¹⁵N Tau-441(2N4R) Uniform Labeled (2 ng/μL, gift from Dr. Guy Lippens, Lille University, France) as an internal standard. Five μL of 500 mM dithiothreitol was added to the suspension, followed by sonication. The resulting solution was mixed with 15 μL of 500 mM iodoacetamide and incubated for 30 minutes at room temperature in the dark. Protein digestion was conducted using the filter-aided sample preparation method as previously reported (Roberts et al., 2020). Briefly, each prepared solution was loaded on a Nanosep 10K filter unit (PALL) and centrifuged. After washing the sample on the filter unit with 8 M urea in 100 mM TEABC solution, immobilized proteins were digested on the filter using 0.25 μg of endoproteinase Lys-C at 37° C. for 60 minutes. Then, the samples were further digested using 0.4 μg trypsin at 37° C. overnight.

The digested samples (soluble and insoluble tau species) were collected by centrifugation, then desalted by C18 TopTip (Glygen). In this purification process, 50 fmol each of AQUA internal-standard peptide for residues 354-369 (MTBR tau-354) and 354-368 (tau368) was spiked for the differential quantification. Before eluting samples, 3% hydrogen peroxide and 3% formic acid (FA) in water were added onto the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system (Waters) coupled to Orbitrap Fusion Tribrid or Orbitrap Tribrid Eclipse (Thermo Scientific) operating in parallel reaction monitoring (PRM) mode.

Sixteen brain tau peptides from both soluble and insoluble tau species were quantified by comparison with corresponding isotopomers signals from the ¹⁵N or AQUA internal standard (Table 4). Peptide-profile comparisons across brain samples were performed by normalizing each peptide amount by a mid-domain tau peptide (residue 181-190).

TABLE 4

| | Tau-441 | |
|---|---|---|
| | residues | |
| Peptide | (Abbreviated) | Sample matrix |
| QEFEVMEDHAGTYGLGDR (SEQ ID NO: 11) | 6-23 | Brain, CSF |
| DQGGYTMHQDQEGDTDAGLK (SEQ ID NO: 12) | 25-44 | Brain, CSF |
| ESPLQTPTEDGSEEPGSETSDAK (SEQ ID NO: 13) | 45-67 | Brain, CSF |
| STPTAEDVTAPLVDEGAPGK (SEQ ID NO: 14) | 68-87 | Brain, CSF |
| QAAAQPHTEIPEGTTAEEAGIGDTPSLEDEAAGHVTQAR | 88-126 | CSF |
| (SEQ ID NO: 15) | | |
| IATPR (SEQ ID NO: 16) | 151-155 | Brain, CSF |
| TPPSSGEPPK (SEQ ID NO: 10) | 181-190 | Brain, CSF |
| SGYSSPGSPGTPGSR (SEQ ID NO: 17) | 195-209 | Brain, CSF |
| TPSLPTPPTR (SEQ ID NO: 18) | 212-221 | CSF |
| VAVVR (SEQ ID NO: 19) | 226-230 | Brain, CSF |
| LQTAPVPMPDLK (SEQ ID NO: 3) | 243-254 | Brain, CSF |
| | (MTBR tau-243) | |
| IGSTENLK (SEQ ID NO: 2) | 260-267 | Brain, CSF |
| VQIINK (SEQ ID NO: 4) | 275-280 | Brain, CSF |
| LDLSNVQSK (SEQ ID NO: 5) | 282-290 | CSF |
| HVPGGGSVQIVYKPVDLSK (SEQ ID NO: 6) | 299-317 | Brain, CSF |
| | (MTBR tau-299) | |
| IGSLDNITHVPGGGNK (SEQ ID NO: 7) | 354-369 | Brain, CSF |
| | (MTBR tau-354) | |
| IGSLDNITHVPGGGN (SEQ ID NO: 8) | 354-368 | Brain, CSF |
| | (tau368) | |
| TDHGAEIVYK (SEQ ID NO: 20) | 386-395 | Brain, CSF |
| SPVVSGDTSPR (SEQ ID NO: 21) | 396-406 | Brain, CSF |

Abbreviations:
microtubule binding region (MTBR),
cerebrospinal fluid (CSF)

CSF tau analysis by MS: CSF (455 μL) was mixed with 10 μL of solution containing ¹⁵N Tau-441(2N4R) Uniform Labeled (100 pg/μL) as an internal standard. The tau species consisting primarily of N-terminal to mid-domain regions were immunoprecipitated with Tau1 and HJ8.5 antibodies. Immunoprecipitated tau species were processed and digested as described previously (Sato et al., 2018). Subsequently, 20 μL of ¹⁵N-tau internal standard (100 pg/μL) was spiked into the post-immunoprecipitated CSF. Then, tau was chemically extracted as previously reported (Barthélemy et al., 2016b) with some modifications. Highly abundant CSF proteins were precipitated using 25 μL of perchloric acid. After mixing and incubation on ice for 15 minutes, the mixture was centrifuged at 20,000 g for 15 minutes at 4° C., and the supernatant was further purified using the Oasis HLB 96-well μElution Plate (Waters) according to the following steps. The plate was washed once with 300 μL of methanol and equilibrated once with 500 μL of 0.1% FA in water. The supernatant was added to the Oasis HLB 96-well μElution Plate and adsorbed to the solid phase. Then, the solid phase was washed once with 500 μL of 0.1% FA in water. Elution buffer (100 μL; 35% acetonitrile and 0.1% FA in water) was added, and the eluent was dried by Speed-vac. Dried sample was dissolved by 50 μL of trypsin solution (10 ng/μL) in 50 mM TEABC and incubated at 37° C. for 20 hours.

After incubation for both immunoprecipitated and chemically extracted samples, each tryptic digest was purified by solid phase extraction on C18 TopTip. In this purification process, 5 fmol each of AQUA internal-standard peptide for residues 354-369 (MTBR tau-354) and 354-368 (tau368) was spiked for the differential quantification. Before eluting samples, 3% hydrogen peroxide and 3% FA in water were added to the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system coupled to Orbitrap Fusion Lumos Tribrid or Orbitrap Tribrid Eclipse mass spectrometer (Thermo Scientific) operating in PRM mode. Nineteen CSF tau peptides were quantified (Table 4). The schematic procedure of CSF tau analysis is described in FIG. 2A.

Statistical analysis: Differences in biomarker values were assessed with one-way ANOVAs, unless otherwise specified. A two-sided p<0.05 was considered statistically significant and corrected for multiple comparisons using Benjamini-Hochberg false discovery rate (FDR) method with FDR set at 5% (Benjamini and Hochberg, 1995). Spearman correlations were used to assess associations between tau biomarkers and cognitive testing measures and tau PET SUVR.

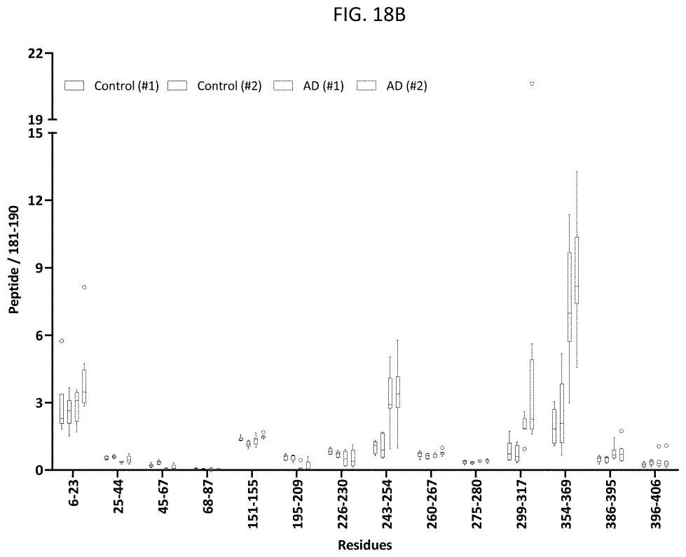

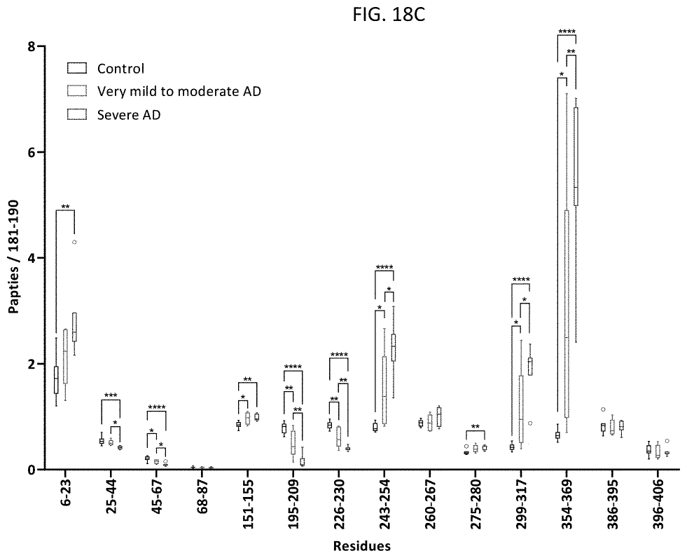

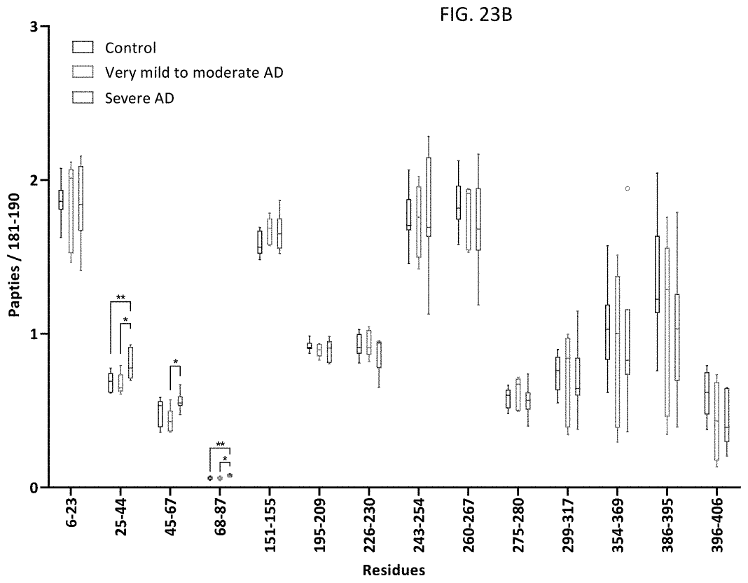

Results—Enrichment profiling of tau species in Alzheimer's disease brain: It was hypothesized that tau aggregation in Alzheimer's disease brain would be reflected in tau profiles in CSF. Therefore, tau profiles in insoluble extracts from Alzheimer's disease and control brains were first analyzed (FIG. 18B: discovery cohort) to later compare with CSF tau profiles. It was determined that the species containing residues 299-317 (MTBR tau-299) and 354-369 (MTBR tau-354), located between R2 and R3 domains and within the R4 domain, respectively, were more enriched in the insoluble extract from Alzheimer's disease brain than control by 3-4 fold. The upstream region of MTBR containing residues 243-254 (MTBR tau-243) was also about three times greater in Alzheimer's disease brain as compared to the control, while species containing residues 260-267 and 275-280 located within R1 and R2 domains, respectively, did not differ between Alzheimer's disease and control tissues. No other regions of tau were enriched in Alzheimer's disease brain compared to controls. Notably, the species containing residue 195-209 within the mid-domain was particularly lower in Alzheimer's disease brain compared to the control, likely the result of extensive hyper-phosphorylation occurring on residues 199, 202, 205, and 208 in insoluble tau aggregates (Malia et al., 2016). Of note, no change was observed for the identified MTBR tau species in soluble tau (whole brain extract) between control and Alzheimer's disease (FIG. 23A). These results were reproduced in brain samples from control (amyloid-negative, n=8), very mild to moderate Alzheimer's disease (amyloid-positive, CDR=0.5−2, n=5) and severe Alzheimer's disease (amyloid-positive, CDR=3, n=7) participants (FIG. 18C and FIG. 23B: validation cohort), which suggested MTBR tau-243, 299, and 354 species were specifically enriched in insoluble tau aggregates over the stages of disease progression.

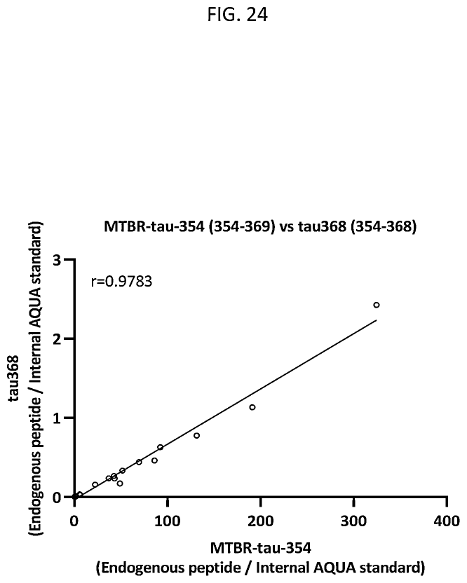

Next, the recently reported truncated tau368 (residue 354-368) species generated by asparagine endopeptidase (Zhang et al., 2014; Blennow et al., 2020) was examined against its paired non-truncated species, MTBR tau-354, and quantified both species in brain insoluble extracts (FIG. 24). A high correlation between tau368 and MTBR tau-354 was found (r=0.9783), suggesting that truncation at residue 368 occurs at the same rate in different stages of brain pathology.

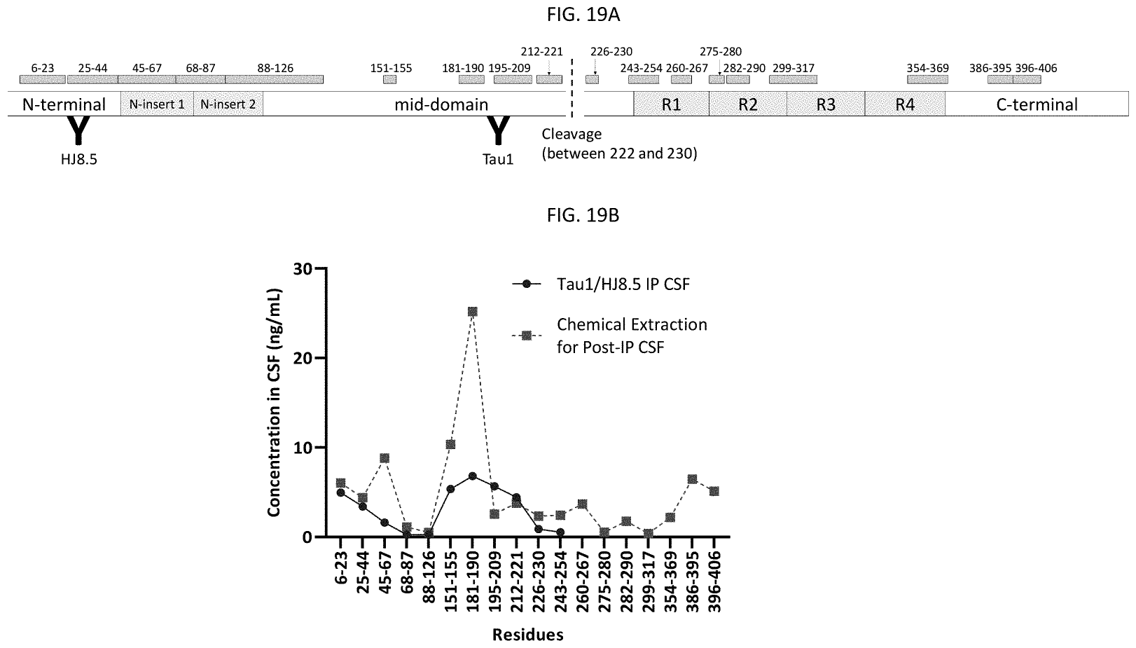

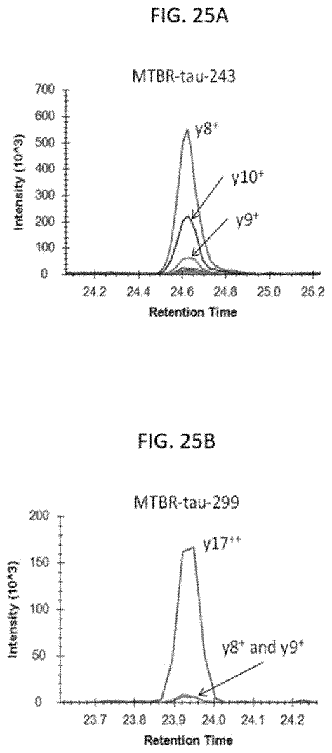

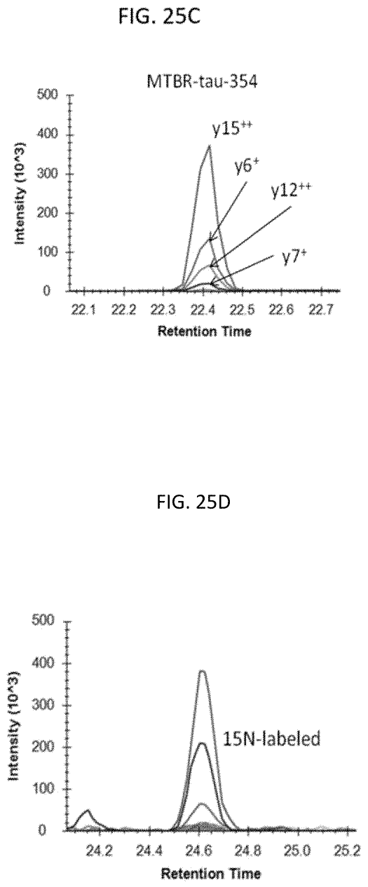

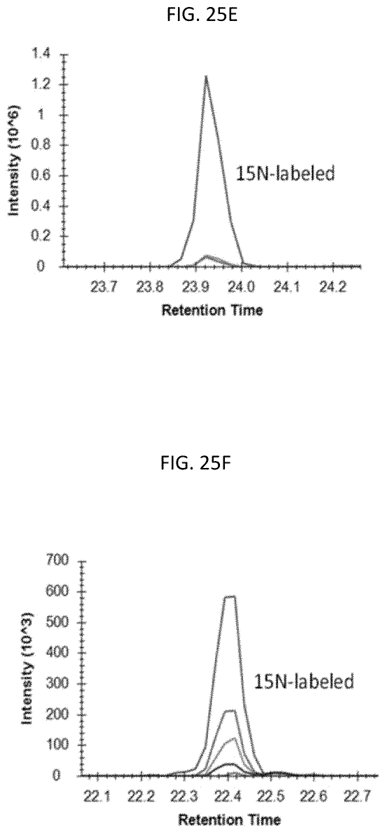

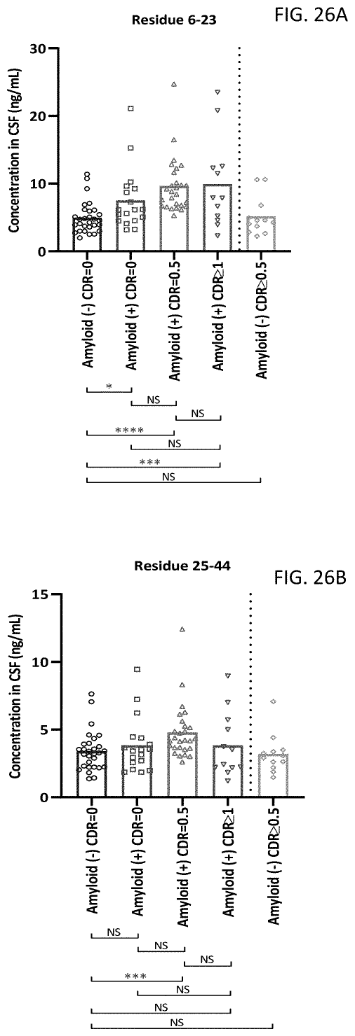

Results—Quantification of MTBR tau in CSF: To determine whether the enrichment of MTBR tau in Alzheimer's disease brain aggregates are related to levels of soluble tau species in the CSF, a method was developed to analyze MTBR tau in CSF. The method utilizes tau chemical extraction in post-immunoprecipitated (Tau1/HJ8.5) CSF followed by MS analysis (FIG. 2A). This method provided sufficient recovery for quantifying MTBR peptides (FIG. 25). Tau peptide abundance recovered by Tau1/HJ8.5 immunoprecipitation method before chemical extraction was dramatically decreased after residue 222 (Sato et al., 2018). In contrast, the concentrations of MTBR tau species quantified by the PostIP-CX method were relatively low compared to the N-terminus to mid domain regions but still comparable to the other regions of tau by immunoprecipitation (FIG. 19). CSF concentrations from normal control participants (calculated as total values from immunoprecipitation and chemical extraction methods) ranged from 8.2 to 32.0 ng/mL for mid-domain species (residues 151-155, 181-190, 195-209, and 212-221), 0.4 to 3.7 ng/mL for MTBR tau species (residues 243-254, 260-267, 275-280, 282-290, 299-317, and 354-369), and 6.5 and 5.1 ng/mL for non-MTBR C-terminal tau species (residues 386-395 and 396-406). The CSF concentrations of C-terminal-containing truncated tau species were in a similar range as those containing the mid-domain (residues 195-209 and 212-221), suggesting the C-terminal side of tau is also truncated in neuronal cells and secreted extracellularly in the same manner as N-terminus to mid-domain tau (Sato et al., 2018).

Results—CSF MTBR tau in an Alzheimer's disease cross-sectional cohort: To determine whether MTBR-containing species present in the extracellular space reflect Alzheimer's disease-related changes, CSF was analyzed from a cross-sectional cohort of amyloid-negative and amyloid-positive participants at different clinical stages: amyloid-negative CDR=0 (control, n=30), amyloid-positive CDR=0 (preclinical AD, n=18), amyloid-positive CDR=0.5 (very mild AD, n=28), amyloid-positive CDR≥1 (mild-moderate AD, n=12), and amyloid-negative CDR≥0.5 (non-AD cognitive impairment, n=12).

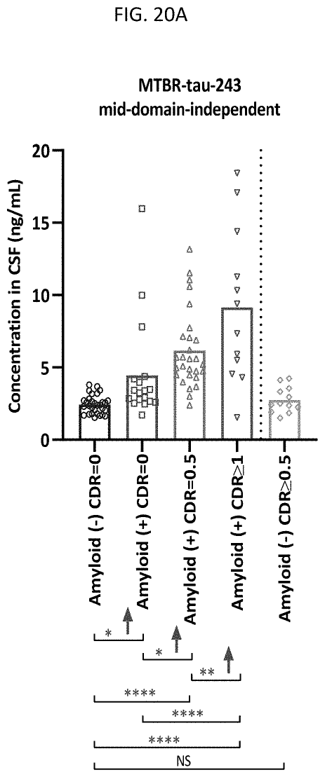

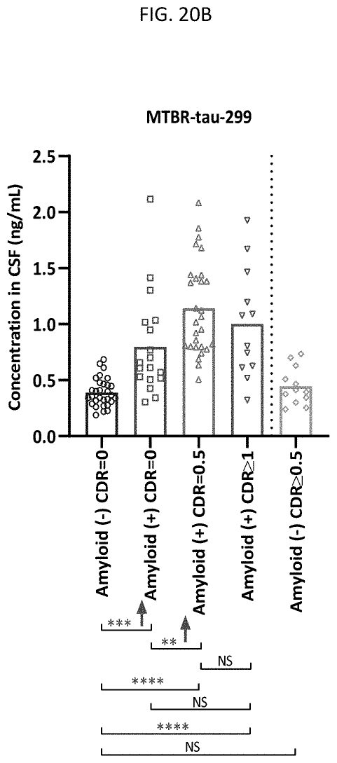

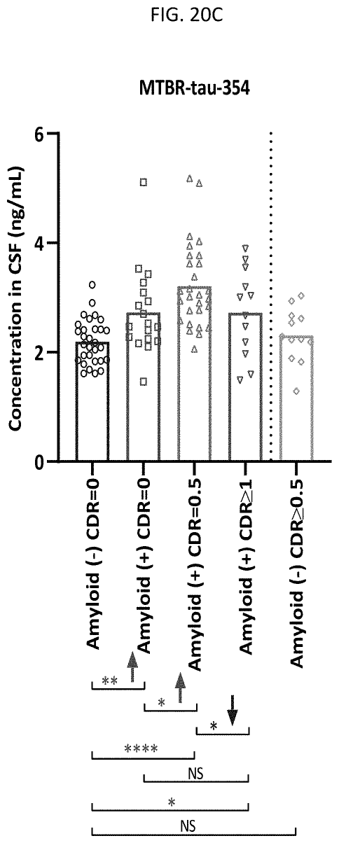

First, CSF levels of three MTBR tau species specifically enriched in Alzheimer's disease brain (MTBR tau-243, MTBR tau-299, and MTBR tau-354) were investigated (FIG. 20). All three species were present in both Alzheimer's disease and control CSF and levels were greater in the amyloid-positive groups even for the asymptomatic stage (CDR=0) when compared to the control group (MTBR tau-243 p=0.0170, MTBR tau-299 p=0.0002, and MTBR tau-354 p=0.0076). Remarkably, these species had distinct characteristics in CSF after clinical disease onset. MTBR tau-299 levels were 204% higher in preclinical AD compared to controls but saturated between very mild AD (CDR=0.5) and mild-moderate AD (CDR≥1) (p=0.2541), while MTBR tau-354 levels were significantly lower in samples collected post-symptom onset (p=0.0345). In contrast, MTBR tau-243 levels were incrementally higher across all disease stages including after symptom onset (p=0.0025). These results suggest that the regional specificity even within the MTBR tau species can distinguish among different Alzheimer's disease stages and that MTBR tau-243 is a good Alzheimer's disease stage-specific marker.

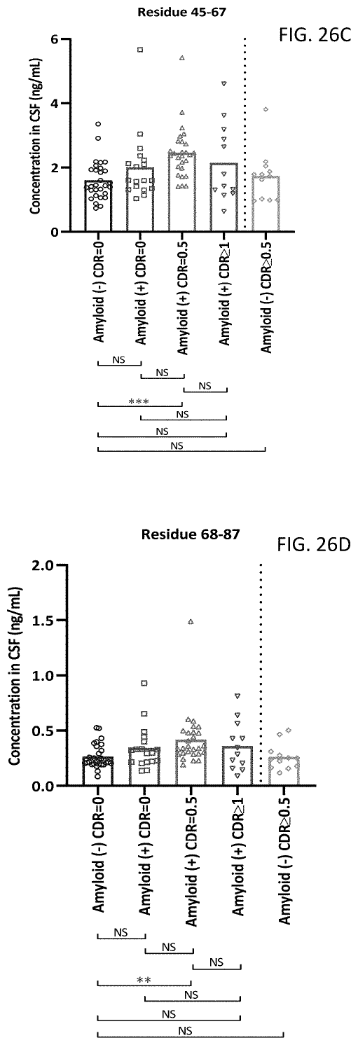

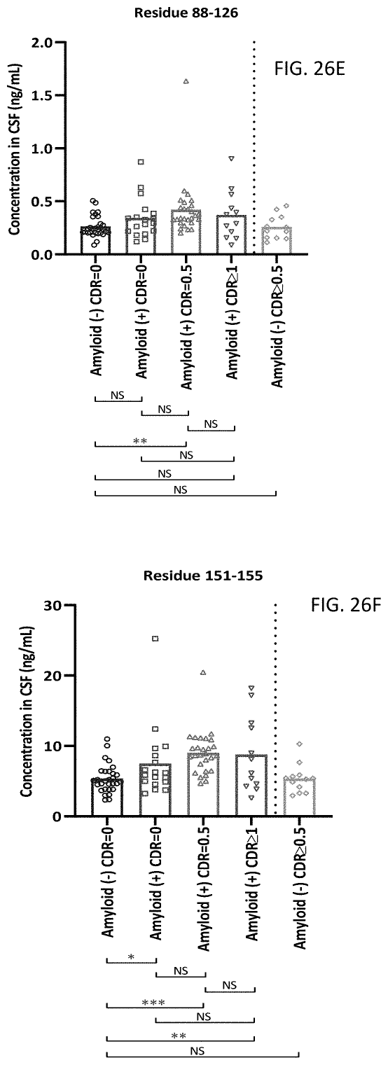

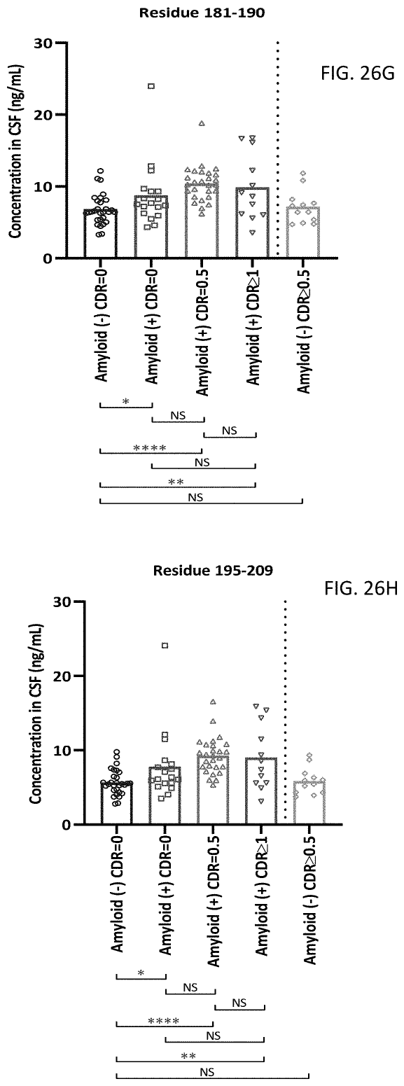

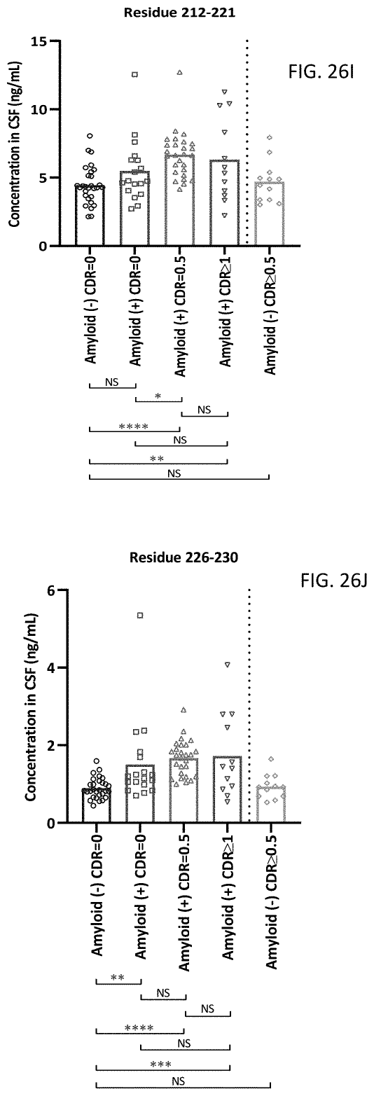

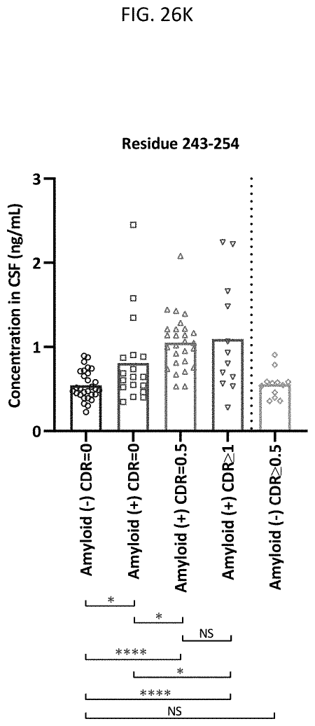

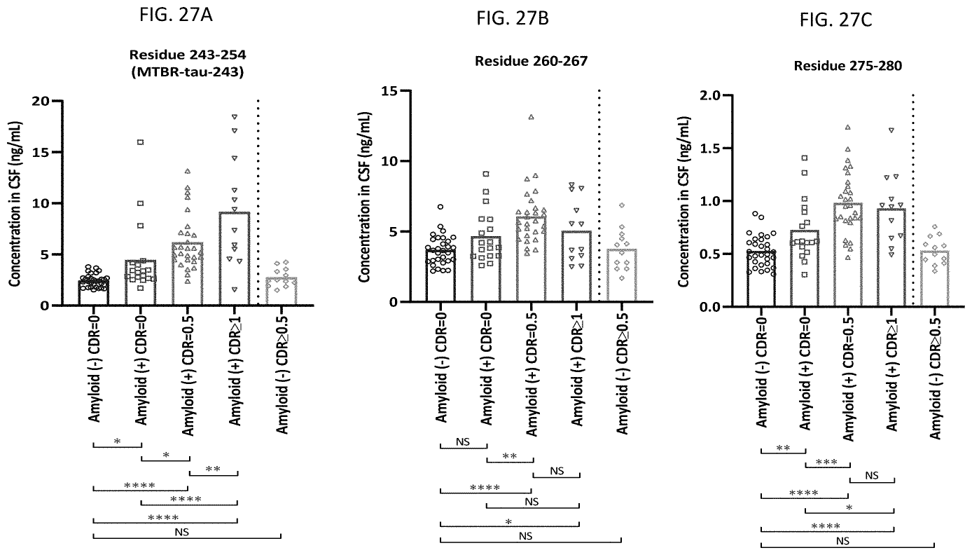

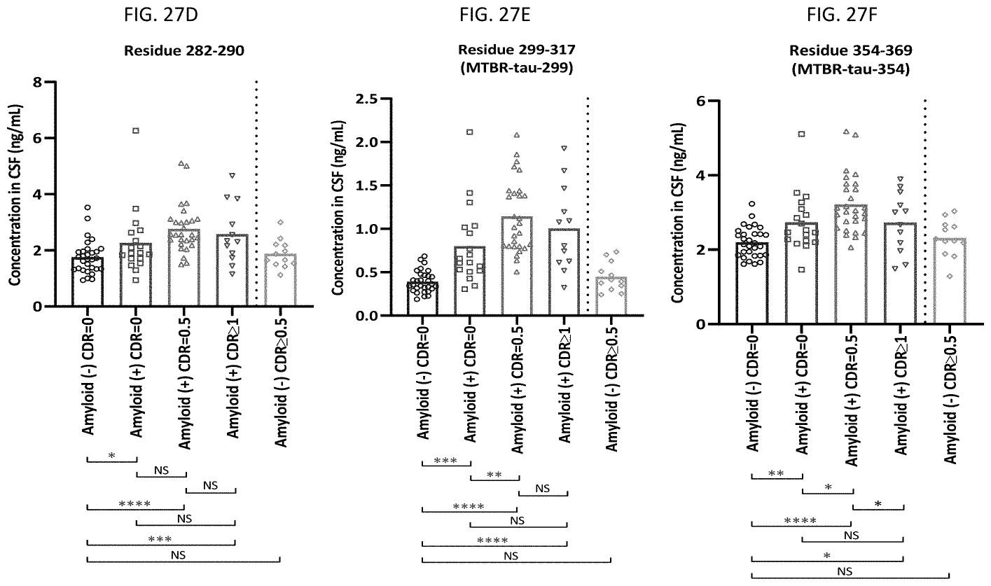

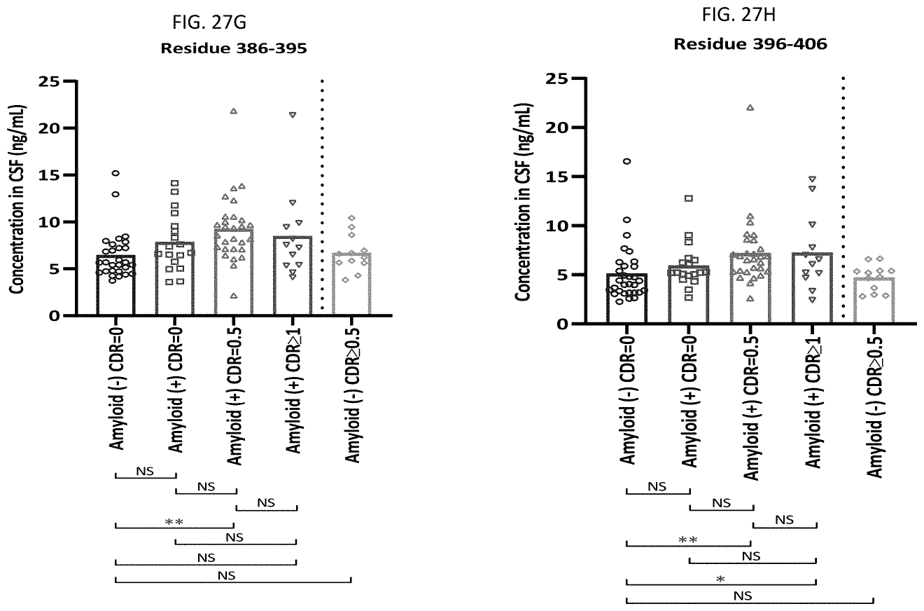

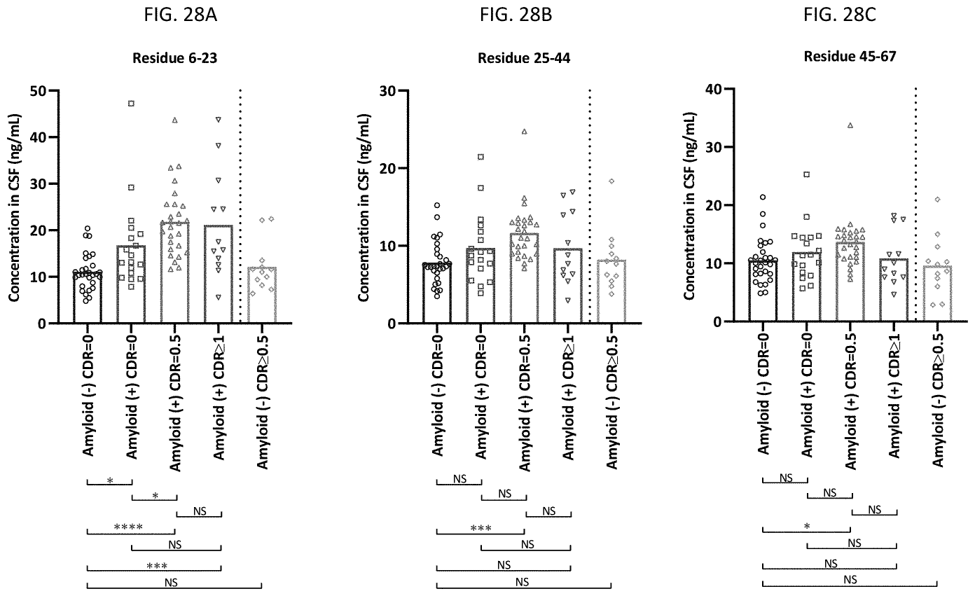

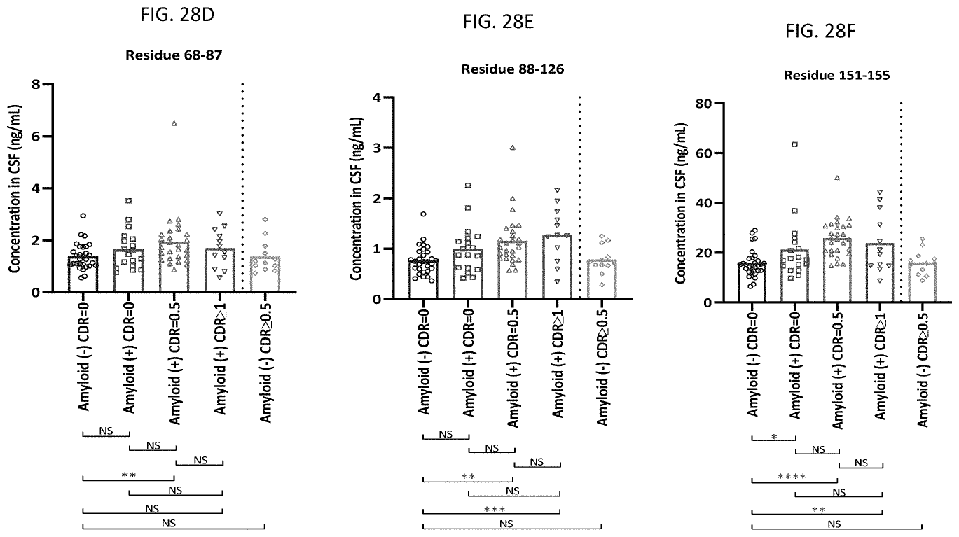

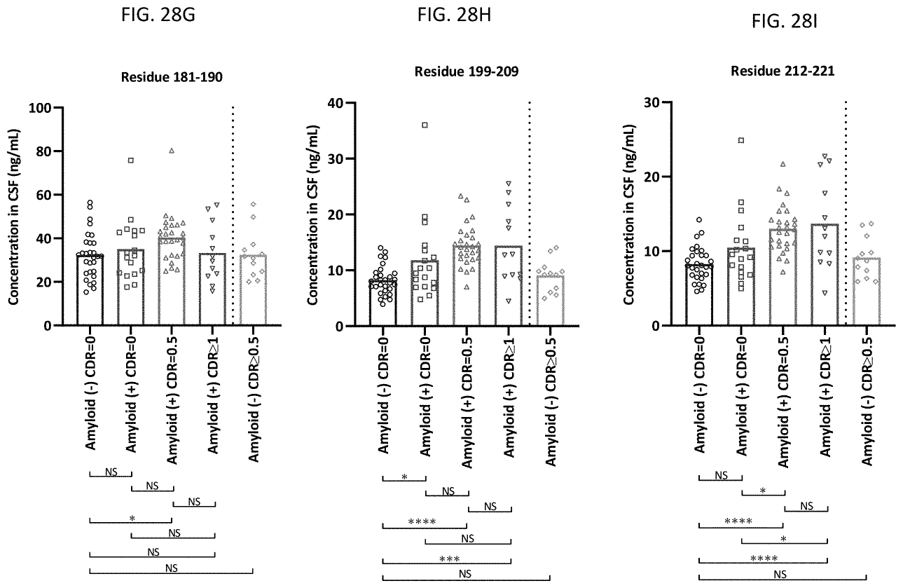

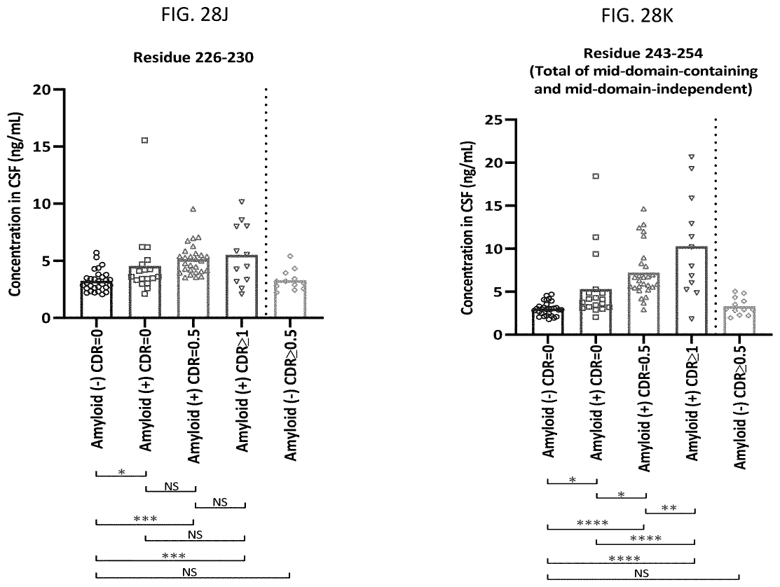

Next investigated was whether CSF MTBR tau species provide enhanced sensitivity and specificity in staging Alzheimer's disease when compared to tau species containing other regions. Multiple species containing N-terminal, mid-domain, MTBR, and C-terminal domains were quantified by region-specific methods (FIG. 26, FIG. 27, FIG. 28). N-terminal and mid-domain species were quantified by immunoprecipitation (IP method) as well as chemical extraction for post-immunoprecipitated CSF (PostIP-CX method), while the MTBR to C-terminal species were quantified only by the chemical extraction method for post-immunoprecipitated CSF (PostIP-CX method) because quantifiable signals were not obtained by immunoprecipitation. Levels of species containing N-terminal domains quantified by the immunoprecipitation method were not different between the control and asymptomatic stage (except for residue 6-23, p=0.0362) or other neighboring disease stages. The mid-domain species levels by the immunoprecipitation method were significantly greater in the asymptomatic amyloid stage than in controls (except for residue 212-221, p=0.0762) but the effect size was relatively modest (123%-168% vs. control) compared to the MTBR tau species (e.g., MTBR tau-299 levels were >200% greater at the preclinical AD stage than control) and did not differ across later disease stages. Regardless of the extraction method, MTBR tau-243, 299, and 354 species showed greater differences between control and disease stages compared to N-terminal to mid-domains species (residues 6-23 to 226-230, FIG. 28). Profiles from the other species containing MTBR to C-terminal domains (residues 260-267, 275-280, 282-290, 386-395, and 396-406) were similar to the mid-domain species and were not specific for the stage of Alzheimer's disease clinical dementia.

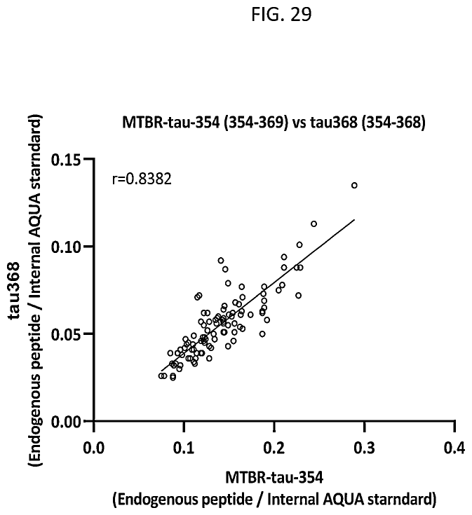

In summary, the three representative species containing MTBR (MTBR tau-243, MTBR tau-299, and MTBR tau-354) that were enriched in Alzheimer's disease brain (FIG. 18) had similar characteristics in CSF with MTBR tau-243 exhibiting the greatest specificity to Alzheimer's disease dementia stage. Of note, a high correlation was observed between the tau368 truncated form and MTBR tau-354 non-truncated form in CSF (r=0.8382) (FIG. 29). The only species that could reliably distinguish the clinical stages of Alzheimer's disease was MTBR tau-243.

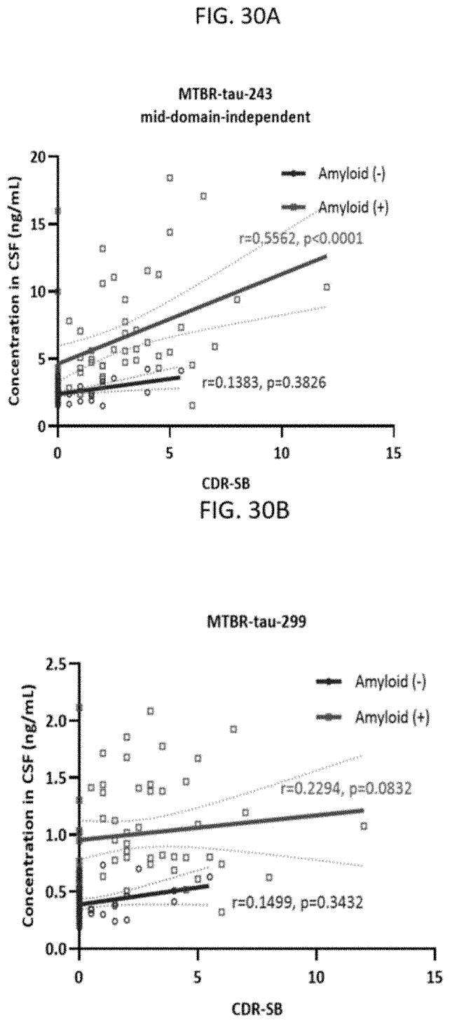

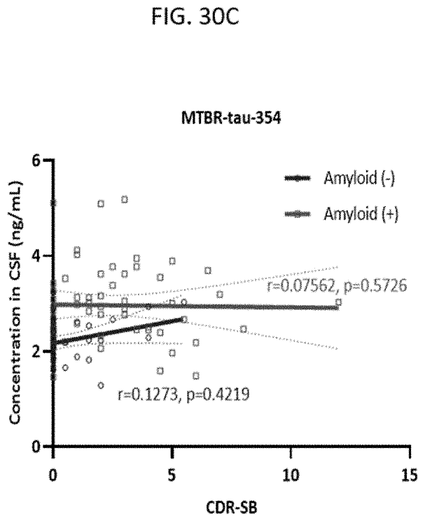

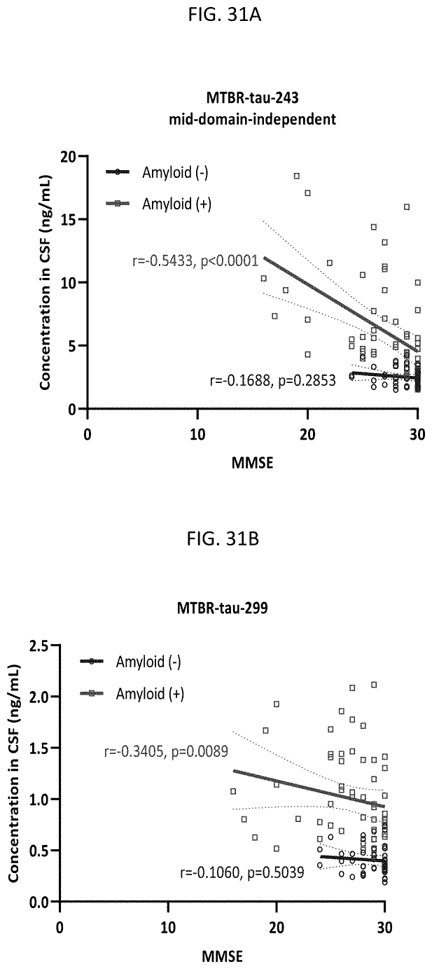

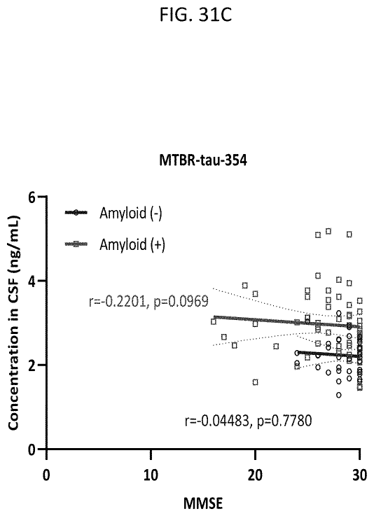

Results—Mid-domain-independent MTBR tau-243 as a specific biomarker to stage Alzheimer's disease: The incrementally greater levels of the MTBR tau-243 species across Alzheimer's disease clinical dementia stages suggest it may be a reliable predictor of disease progression. Next investigated was which MTBR tau species (MTBR tau-243, MTBR tau-299, and MTBR tau-354) had the highest correlations with results of cognitive tests such as CDR-sum of boxes (DR-SB) and the Mini-Mental State Exam (MMSE). It was found that the mid-domain-independent MTBR tau-243 species in the amyloid-positive group was highly correlated with both CDR-SB and MMSE (r=0.5562, p<0.0001 and r=−0.5433, p<0.0001, respectively) (FIG. 30 and FIG. 31). Other species levels had much lower or no significant correlations with the cognitive testing (Table 5), which suggests that CSF MTBR tau-243 specifically differentiates clinical stage and global disease progression from the asymptomatic stage through advancing clinical stages of Alzheimer's disease.

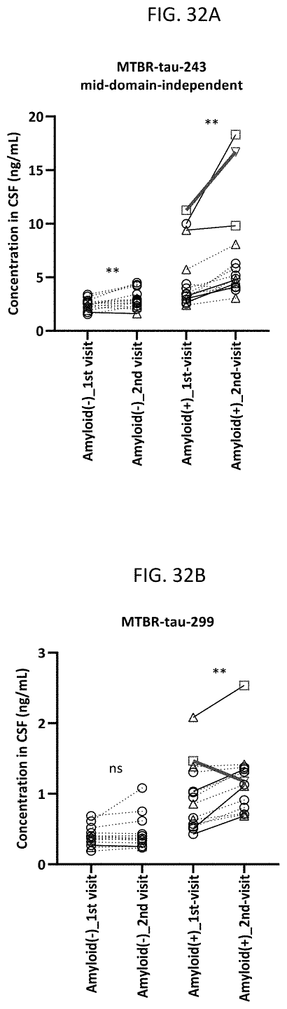

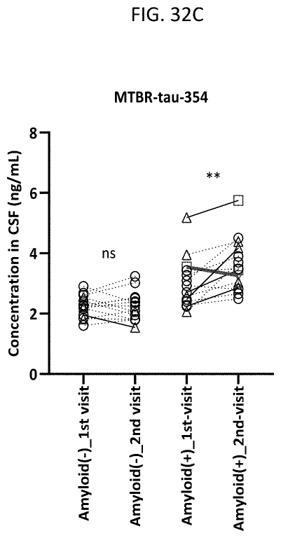

Results—CSF MTBR tau in an Alzheimer's disease longitudinal cohort: From the cross-sectional cohort, a subset of participants (n=28) were followed for two to nine years to measure the longitudinal trajectory of MTBR tau in CSF (Table 6). MTBR tau species enriched in Alzheimer's disease brain (MTBR tau-243, MTBR tau-299, and MTBR tau-354) were significantly increased over time in the amyloid-positive group (p<0.01 by two-tailed paired t-test between 1^{st} and 2^{nd} visits) but not the amyloid-negative group, except for MTBR tau-243 (FIG. 32). The amyloid-negative group also showed slight longitudinal increases of MTBR tau-243 but lower than observed for the amyloid-positive group (means of differences=0.4926 and 2.208 in amyloid-negative and positive groups, respectively).

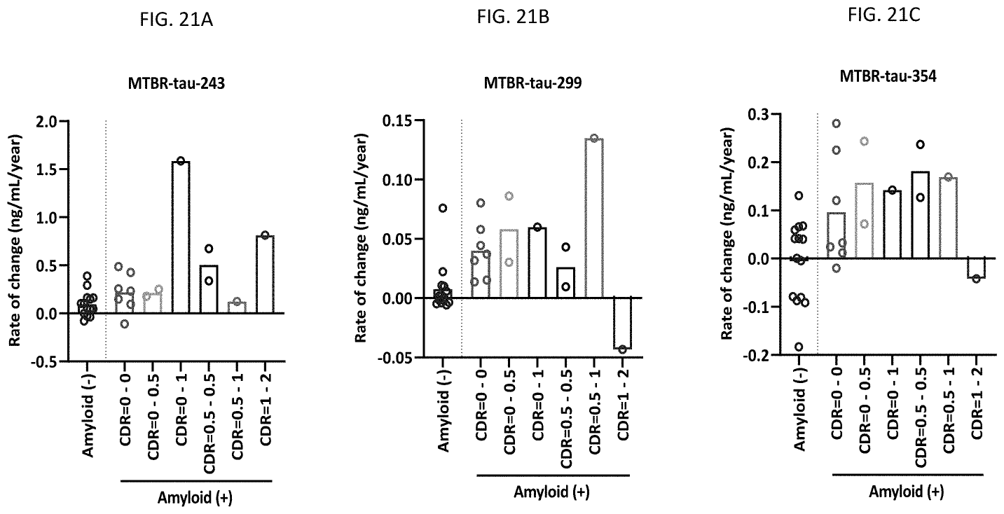

FIG. 21 shows the longitudinal change-rates of the MTBR tau species concentrations in individual participants. Notably, one participant (participant A) with the highest CDR after disease onset (changed from CDR=1 to 2 in seven years) showed specific trajectory profiles for each MTBR tau species. MTBR tau-243 continuously increased even from mild AD (CDR=1) to moderate AD (CDR=2), while MTBR tau-299 and MTBR tau-354 showed a decrease in this participant's CSF after mild AD. Other participants in the amyloid-positive group were classified as preclinical AD or very mild AD (CDR=0 or 0.5, respectively) at the 1^{st} visit, and the increasing trend for each species level was seen for most of the participants, which supports the findings from the cross-sectional cohort.

TABLE 6

| | | Visit | | | MTBR tau-243 | | MTBR tau-299 | | MTBR tau-354 | |
|---|---|---|---|---|---|---|---|---|---|---|
| Participant | Amyloid | interval | CDR | | (ng/mL) | | (ng/mL) | | (ng/mL) | |
| ID | status | (year) | Visit 1 | Visit 2 | Visit 1 | Visit 2 | Visit 1 | Visit 2 | Visit 1 | Visit 2 |
| 1 | Negative | 7.0 | 0.5 | 0 | 1.916 | 2.645 | 0.241 | 0.254 | 1.824 | 2.106 |
| 2 | Negative | 4.9 | 0 | 0 | 2.220 | 2.043 | 0.317 | 0.300 | 2.179 | 1.794 |
| 3 | Negative | 4.5 | 0.5 | 0 | 2.245 | 2.728 | 0.395 | 0.404 | 2.236 | 2.535 |
| 4 | Negative | 8.0 | 0 | 0 | 2.569 | 2.560 | 0.446 | 0.430 | 1.841 | 1.805 |
| 5 | Negative | 5.2 | 0 | 0 | 2.705 | 2.946 | 0.366 | 0.341 | 2.252 | 2.256 |
| 6 | Negative | 5.3 | 0 | 0 | 2.588 | 2.817 | 0.399 | 0.403 | 2.289 | 2.506 |
| 7 | Negative | 6.2 | 0 | 0 | 3.386 | 4.306 | 0.612 | 1.080 | 2.610 | 3.026 |
| 8 | Negative | 6.2 | 0 | 0 | 3.205 | 4.200 | 0.685 | 0.752 | 2.904 | 2.364 |
| 9 | Negative | 5.7 | 0 | 0 | 2.550 | 2.866 | 0.399 | 0.366 | 2.419 | 1.955 |
| 10 | Negative | 4.7 | 0 | 0 | 1.545 | 2.322 | 0.188 | 0.235 | 1.612 | 1.809 |
| 11 | Negative | 4.3 | 0 | 0 | 2.822 | 4.494 | 0.519 | 0.616 | 2.676 | 3.239 |
| 12 | Negative | 4.4 | 0 | 0.5 | 1.729 | 1.604 | 0.269 | 0.252 | 1.948 | 1.546 |
| 13 | Negative | 3.6 | 0.5 | 0 | 2.372 | 3.421 | 0.345 | 0.365 | 2.187 | 2.400 |
| 14 | Negative | 2.6 | 0.5 | 0.5 | 2.432 | 2.229 | 0.376 | 0.372 | 2.539 | 2.062 |
| 15 | Positive | 5.2 | 0 | 1 | 9.992 | 18.307 | 1.034 | 1.347 | 2.704 | 3.449 |
| 16 | Positive | 8.6 | 0 | 0.5 | 3.234 | 4.752 | 0.425 | 0.685 | 2.241 | 2.856 |
| 17 (participant | Positive | 6.7 | 1 | 2 | 11.253 | 16.698 | 1.466 | 1.177 | 3.546 | 3.265 |
| A) | | | | | | | | | | |
| 18 | Positive | 3.5 | 0.5 | 0.5 | 5.722 | 8.071 | 1.383 | 1.416 | 3.948 | 4.390 |
| 19 | Positive | 7.0 | 0 | 0.5 | 2.665 | 4.417 | 0.505 | 1.109 | 2.469 | 4.180 |
| 20 | Positive | 6.1 | 0 | 0 | 3.470 | 4.055 | 0.530 | 0.803 | 2.930 | 2.809 |
| 21 | Positive | 9.1 | 0 | 0 | 2.529 | 4.852 | 0.570 | 0.708 | 2.278 | 2.497 |
| 22 | Positive | 7.9 | 0 | 0 | 2.926 | 6.297 | 0.658 | 0.910 | 2.404 | 2.663 |
| 23 | Positive | 5.0 | 0 | 0 | 3.424 | 5.866 | 1.018 | 1.309 | 3.093 | 4.504 |
| 24 | Positive | 7.3 | 0.5 | 0 | 3.002 | 4.092 | 0.857 | 1.128 | 2.061 | 3.704 |
| 25 | Positive | 5.2 | 0 | 0 | 3.969 | 5.171 | 1.303 | 1.375 | 3.270 | 3.895 |
| 26 | Positive | 5.0 | 0 | 0 | 4.380 | 3.829 | 0.949 | 1.350 | 3.427 | 3.486 |
| 27 | Positive | 3.3 | 0.5 | 1 | 9.399 | 9.809 | 2.084 | 2.535 | 5.182 | 5.748 |
| 28 | Positive | 2.0 | 0.5 | 0.5 | 2.397 | 3.059 | 0.634 | 0.719 | 2.581 | 3.045 |

Abbreviations: Clinical Dementia Rating (CDR)

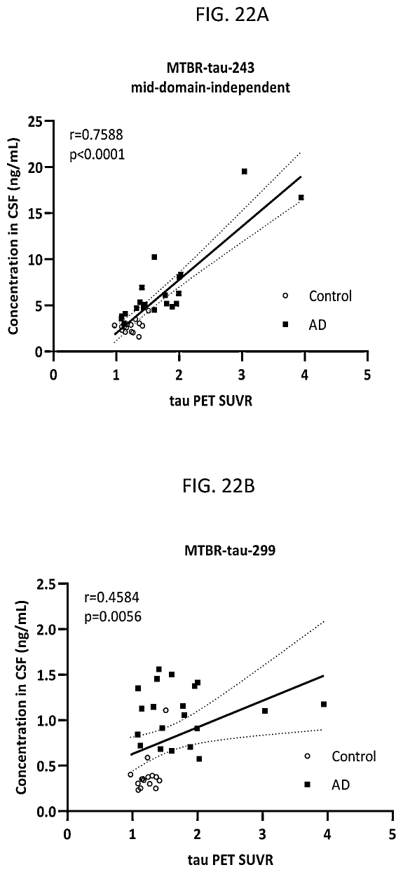

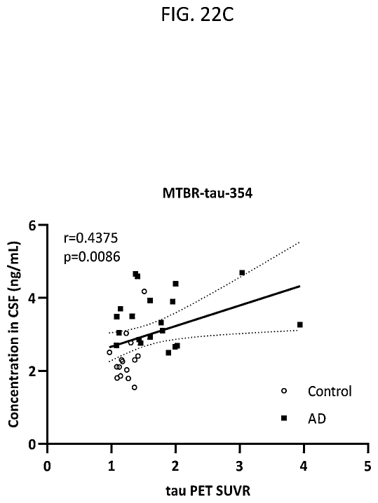

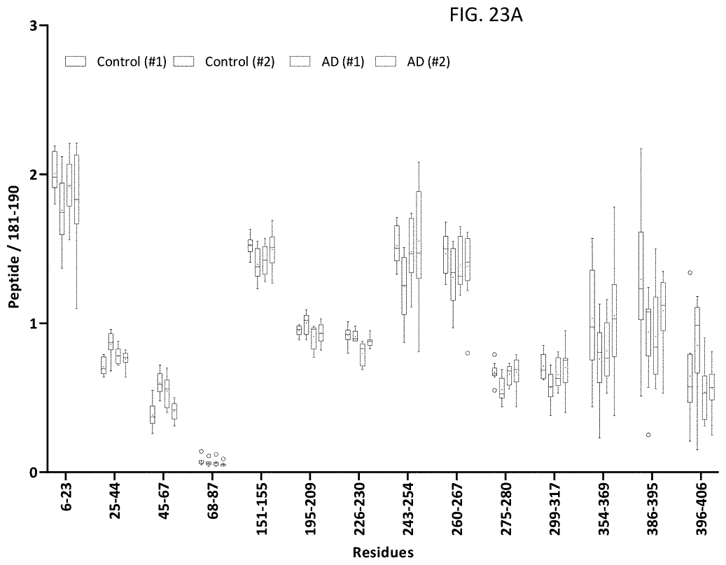

Results—Correlation with tau PET imaging: Tau pathology as measured by tau PET scans correlates strongly with cognitive decline and clinical stage of Alzheimer's disease (Arriagada et al., 1992; Johnson et al., 2016; Ossenkoppele et al., 2016; Bejanin et al, 2017; Jack et al, 2018; Gordon et al, 2019). Next investigated was whether MTBR tau in CSF was correlated with brain tau pathology measured by tau PET (FIG. 22). Mid-domain-independent MTBR tau-243 significantly correlated with tau PET SUVR (r=0.7588, p<0.0001), while MTBR tau-299 and MTBR tau-354 were much less correlated (r=0.4584, p=0.0056 and r=0.4375, p=0.0086, respectively). Tau species containing residue 226-230 also showed high correlation with tau PET SUVR (r=0.6248, p<0.0001, Table 7), but lower than observed for MTBR tau-243. This suggests that CSF MTBR tau-243 and the surrounding region may be surrogate biomarkers of tau aggregation in the brain. The ability to specifically and quantitatively track tau pathology in the brain is a much needed biomarker for Alzheimer's disease clinical studies.

TABLE 7
Correlations between each CSF tau species and tau PET
SUVR: mid-domain-independent MTBR tau-243 correlates
with tau pathology better than the other tau species

| Residue | | | |
|---|---|---|---|
| (Abbreviated) | Preparation method | r | p-value |
| 6-23 | IP | 0.5361 | 0.0011 |
| 25-44 | IP | 0.4411 | 0.0090 |
| 45-67 | IP | 0.5276 | 0.0013 |
| 68-87 | IP | 0.4700 | 0.0050 |
| 88-126 | IP | 0.3928 | 0.0215 |
| 151-155 | IP | 0.5501 | 0.0006 |
| 181-190 | IP | 0.5315 | 0.0010 |
| 195-209 | IP | 0.5471 | 0.0007 |
| 212-221 | IP | 0.4779 | 0.0037 |
| 226-230 | IP | 0.6248 | <0.0001 |
| 243-254 | IP | 0.6346 | <0.0001 |
| 243-254 | PostIP-CX | 0.7588 | <0.0001 |
| (MTBR tau-243) | | | |
| 260-267 | PostIP-CX | 0.3787 | 0.0249 |
| 275-280 | PostIP-CX | 0.5263 | 0.0012 |
| 282-290 | PostIP-CX | 0.5003 | 0.0022 |
| 299-317 | PostIP-CX | 0.4584 | 0.0056 |
| (MTBR tau-299) | | | |
| 354-369 | PostIP-CX | 0.4375 | 0.0086 |
| (MTBR tau-354) | | | |
| 386-395 | PostIP-CX | 0.4139 | 0.0185 |
| 396-406 | PostIP-CX | 0.3843 | 0.0248 |

Discussion: MTBR regions of tau have been investigated primarily in brain aggregates but not extensively in CSF. In this study, using a sensitive and antibody-independent method to analyze CSF tau, the presence and quantification of MTBR regions of tau in CSF samples from human participants was shown. Past studies utilizing antibody-dependent assays (Meredith et al., 2013; Sato et al., 2018) may have failed to detect MTBR-containing tau species in CSF due to assay limitations including antibody specificity or sensitivity, or the ability to recover potential conformations adopted by MTBR species in CSF. Alternatively, MTBR tau may be truncated by various proteases, generating fragments that are not detected in conventional immunoassays or immunoprecipitation followed by MS assays (Gamblin et al., 2003; Cotman et al., 2005; Zhang et al., 2014; Zhao et al., 2016; Chen et al., 2018; Quinn et al., 2018). In this study, surprisingly robust concentrations of MTBR tau species were measured, at about 1% to 10% compared to the mid-domain tau species, by using a PostIP-CX method followed by mass spectrometry (FIG. 19 and FIG. 2A).

To date, it has been unclear whether MTBR tau could be involved in extracellular tau propagation because extracellular levels were thought to be too low to seed and spread the pathology. These new findings of the stoichiometry of MTBR in CSF support the hypothesis that MTBR-containing species could spread extracellularly as pathological species. These measures also inform potential targets of anti-tau drugs in development for Alzheimer's disease and provide a quantitative measure of the target as shown by the two-fold to three-fold increase of MTBR tau species in CSF from Alzheimer's disease patients. However, a limitation is that the pathological species may be present in the interstitial fluid (ISF) rather than CSF (Colin et al., 2020). Although some reports revealed that CSF tau originates mainly from ISF (Reiber, 2001) and human CSF from Alzheimer's disease patients can induce tau seeding in a transgenic mice model (Skachokova et al., 2019), further investigations are necessary to address if tau species detected in CSF reflect pathological tau which can propagate in human brain.

Previous studies show that inoculation with Alzheimer's disease brain tau aggregates into mouse brain induced severe tau pathology (Guo et al., 2016; Narasimhan et al., 2017); however, there have been no reports that identify the pathological tau species within the extracellular space that is also linked to disease progression in humans. This led to the testing of whether CSF MTBR tau species change in Alzheimer's disease, and exploration of their suitability as novel Alzheimer's disease biomarkers. It was found that CSF MTBR tau levels are elevated in Alzheimer's disease and are consistent with species enriched in Alzheimer's disease brain insoluble fractions. The finding that CSF MTBR tau correlates with Alzheimer's disease clinical stage and tau pathology suggests that MTBR tau is related to the mechanism of tau propagation in Alzheimer's disease, although the nature (i.e., monomeric, oligomeric, or fibril species) and origin of extracellular CSF MTBR tau are still unknown. It is possible that CSF MTBR tau may originate from brain aggregates or from neurons that actively secrete a monomeric species, and future studies should be designed to address this issue.

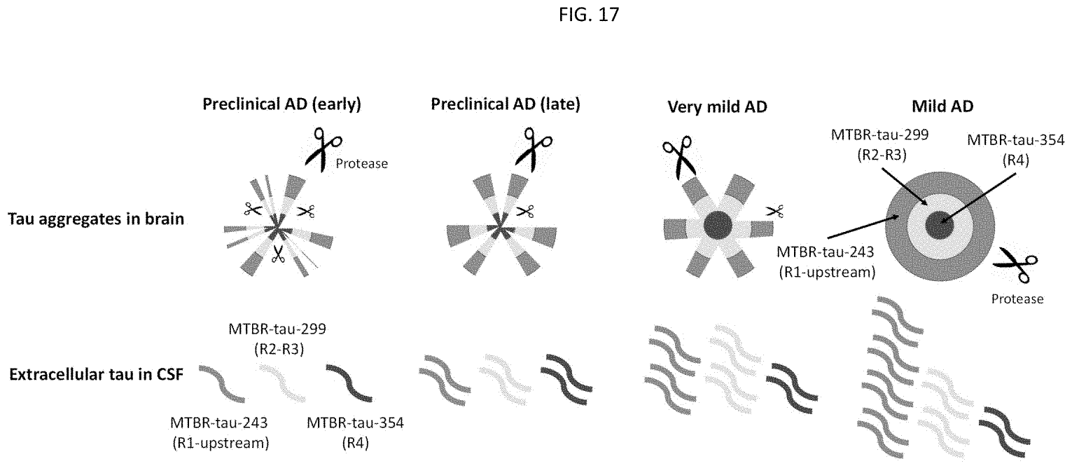

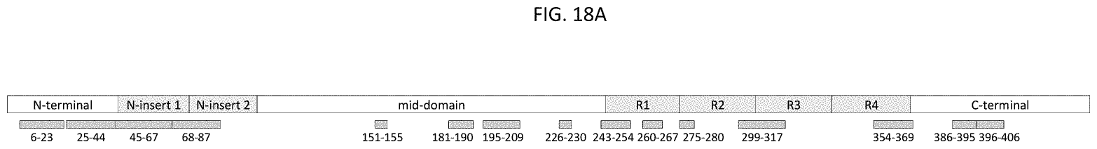

Interestingly, the trajectories of the change in CSF MTBR tau species were found to be distinct across different regions of the MTBR and at each clinical stage of Alzheimer's disease. We posit that this finding is due to structural changes in tau as determined by recent Cryo-EM findings. Cryo-EM analysis suggests the ordered β-sheet core of tau aggregates starts at residue 306 (Fitzpatrick et al., 2017). Thus, MTBR tau-354 (containing residue 354-369), MTBR tau-299 (containing residue 299-317) and MTBR tau-243 (containing residue 243-254) represent the internal side, border, and external side of the filament core, respectively. In contrast to both MTBR tau-354 and MTBR tau-299, MTBR tau-243 levels were incrementally greater across all disease stages. MTBR tau-243 and the nearby region (i.e., residue 226-230) levels in CSF are also highly correlated with tau PET SUVR performance (FIG. 22 and Table 7), which supports the hypothesis that MTBR tau-243 and potentially the nearby region deposit into brain tau aggregates and are also secreted extracellularly (FIG. 17).

The findings that MTBR tau highly correlates with Alzheimer's disease pathology and clinical progression stages provide important insights into promising targets for therapeutic anti-tau drugs to treat tauopathies. For example, a novel tau antibody, recognizing an epitope in the upstream region of MTBR (residue 235-250) demonstrated a significant and selective ability to mitigate tau seeding from Alzheimer's disease and progressive supranuclear palsy brains in cell-based assays (Courade et al., 2018). These findings suggest that the upstream region of MTBR could be related to extracellular, pathological tau. This is supported by the antibody mitigated propagation of tau pathology to distal brain regions in transgenic mice that had been injected with human Alzheimer's disease brain extracts (Albert et al., 2019). Another novel tau antibody, recognizing an epitope in the upstream region of MTBR (residue 249-258) demonstrated the reduction of inducing tau pathology in cellular- and in vivo transgenic mice models seeded by human Alzheimer's disease brain extracts (Vandermeeren et al., 2018). Antibodies targeting MTBR tau-299 and MTBR tau-354 species also mitigated tau pathology induced by seeding of P301L tau or Alzheimer's disease brain extract (Weisová et al., 2019; Roberts et al., 2020), which supports the hypothesis that species containing specific regions of MTBR are responsible for the spread of tau pathology in tauopathies.

In summary, it was discovered that MTBR tau species in CSF exist as C-terminal fragments and are specifically increased in Alzheimer's disease, reflecting the enrichment seen in Alzheimer's disease brain aggregates. The findings suggest specific MTBR-containing species (MTBR tau-299 and MTBR tau-243) are promising CSF biomarkers to measure amyloid and tau pathology in Alzheimer's disease. In particular, the mid-domain-independent MTBR tau-243 paralleled disease progression and tau pathology in Alzheimer's disease and may be utilized as a biomarker of tau pathology and a target for novel anti-tau antibody therapies.

### Example 4

An additional sample processing method, referred to as “PostIP-IP”, was developed and compared to the PostIP-CX method described in Examples 1 and 2. An exemplary workflow of the PostIP-IP method is provided in FIG. 33.

CSF samples obtained from the LOAD100 cohort described in Example 2 were processed by the PostIP-CX method (Example 1) or the PostIP-IP method (this example) and then analyzed by LC-MS as generally described in Example 2.

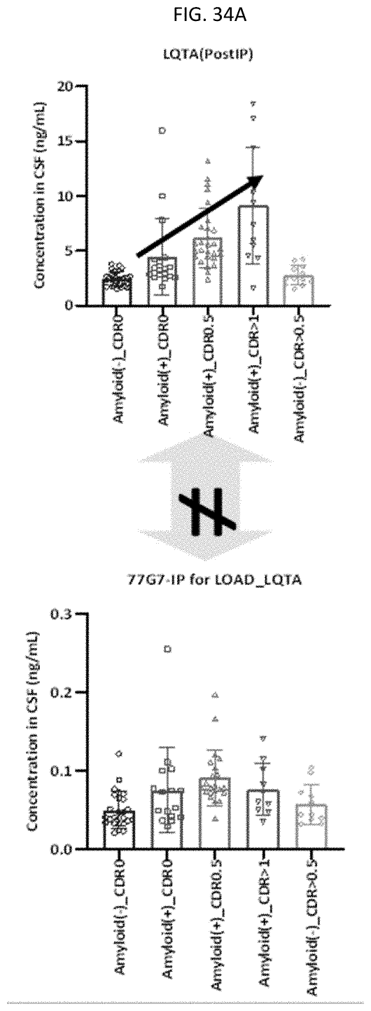

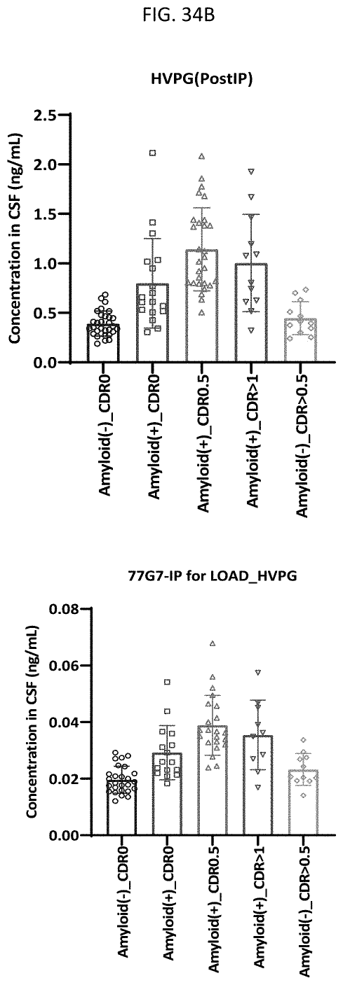

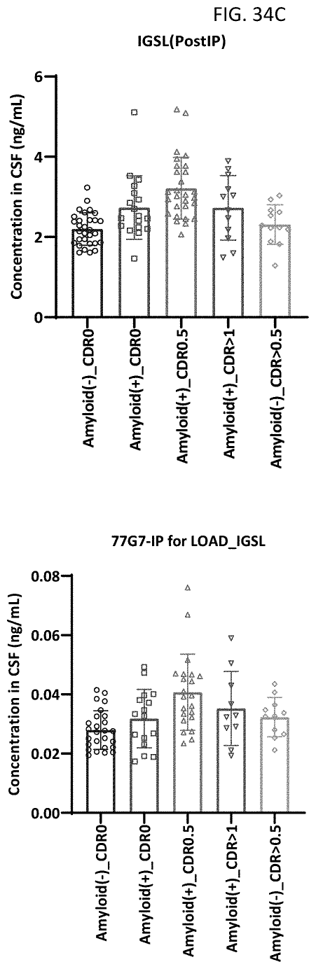

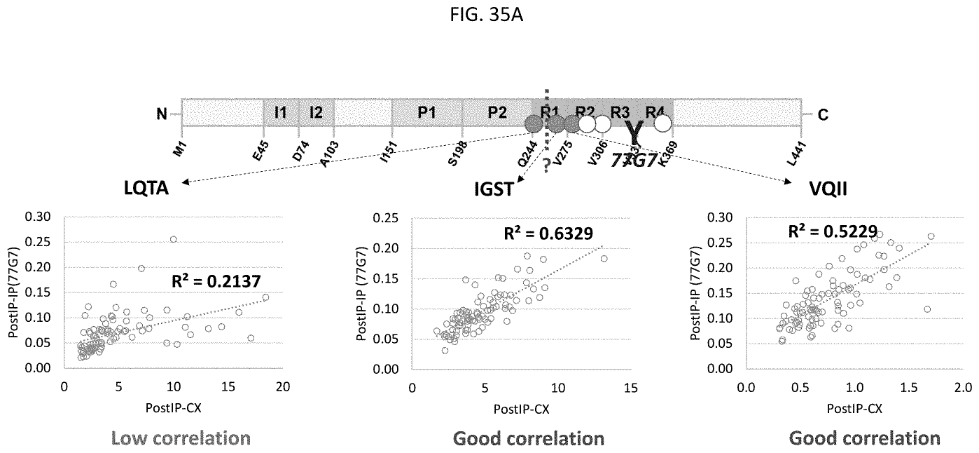

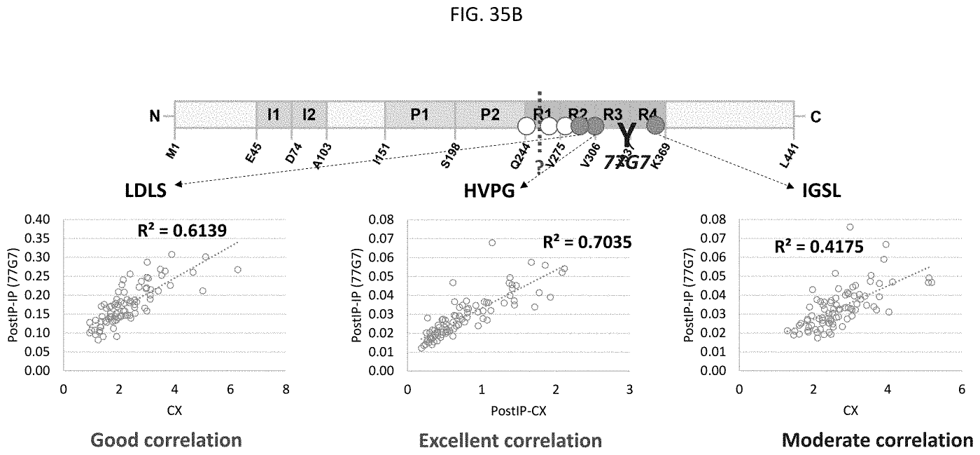

As shown in FIG. 34A, the tryptic peptide LQTA showed a different profile between the two samples. For instance, the continuous increase in the amount of LQTA, even after clinical onset, measured in samples processed by the PostIP-CX method was not observed in samples processed by the PostIP-IP method. In contrast, the tryptic peptides HVPG and IGSL show similar profiles between samples processed by the PostIP-CX and PostIP-IP methods (FIG. 34B and FIG. 34C). Further analysis of additional tryptic peptides suggests there may be an important cleavage event occurring in R1 within the amino acid sequence between the LQTA and IGST peptides (FIG. 35A).

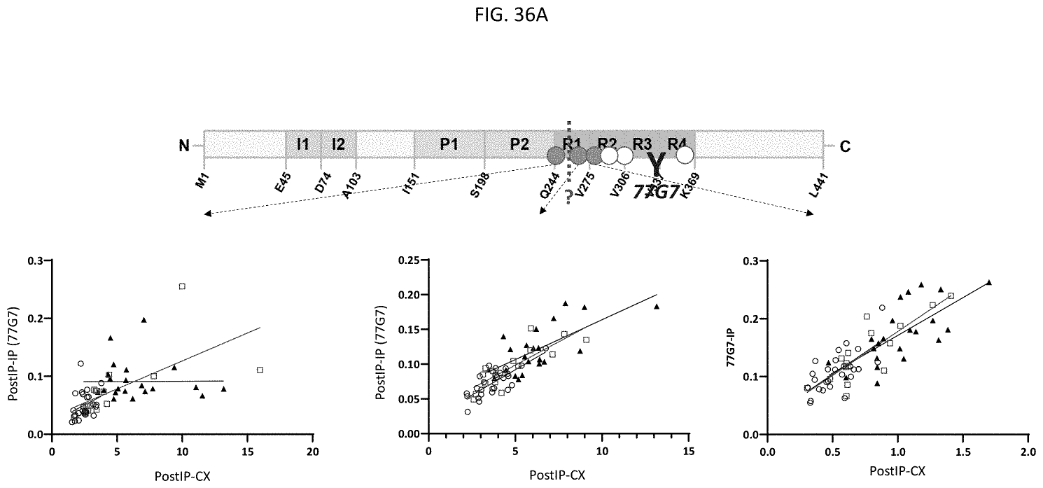

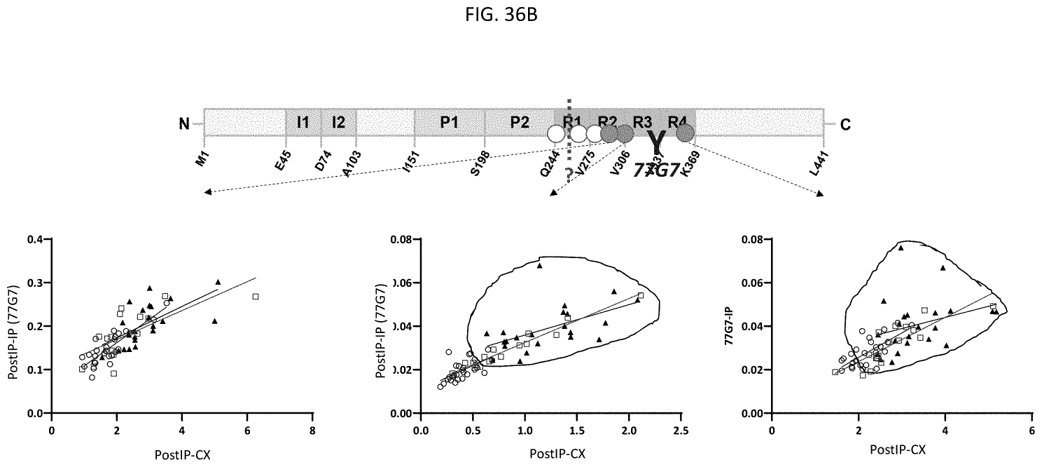

Although the abundance of all tryptic peptides downstream (i.e., C-terminal) to LQTA showed better correlation between sample processing methods (based on R² value, see FIG. 35), there was a notable decrease in the R² value between the tryptic peptides HVPG and IGSL. To explore this further, the samples were grouped by CDR score—more specifically, cognitive-impaired subjects (CI, CDR>0.5) and cognitive unimpaired subjects (CDR<0.5). As shown in FIG. 36B, only the cognitive-impaired subjects showed a low correlation between samples processed by the PostIP-CX vs. PostIP-IP method for the HVPG and IGSL tryptic peptides. This may reflect the occurrence of tau-aggregation in the brain, which would recruit regions of tau comprising the HVPG and IGSL tryptic peptides into the aggregates thereby leading to changing amounts of tau species comprising these peptides in CSF and other biological fluids.

Overall, these data indicate that the choice of sample processing method affects the ability to detect, in CSF and other biological fluids, MTBR tau species that recapitulate tau pathology in the CNS.

### Example 5

In this example, CSF samples from three clinical cohorts of subjects were processed by the IP method (Example 1) and the PostIP-IP method (Example 4) and evaluated by mass spectrometry as generally described in Example 3. The samples were obtained from control subjects (n=93), amyloid positive subjects with AD (n=41), subjects with non-AD tauopathies (n=87). The subjects with non-AD tauopathies were clinically diagnosed with CBD or CBD/PSP (n=20), FTD (n=29), FTLD (R406W n=7, P301L n=3), PSP (n=18), and non-AD dementia, not defined (n=3). Tryptic peptides specific to 3R and 4R isoforms were of particular interest. CSF Aβ42/40 was measured by mass spectrometry as generally described in Ovod et al., Alzheimers Dement J. Alzheimers Assoc, 2017, 13:841-849. Amyloid status was defined using a cut-off value of 0.085 (i.e., amyloid positive >0.085, amyloid negative <0.085). pT217% was measured by mass spectrometry as described previously.

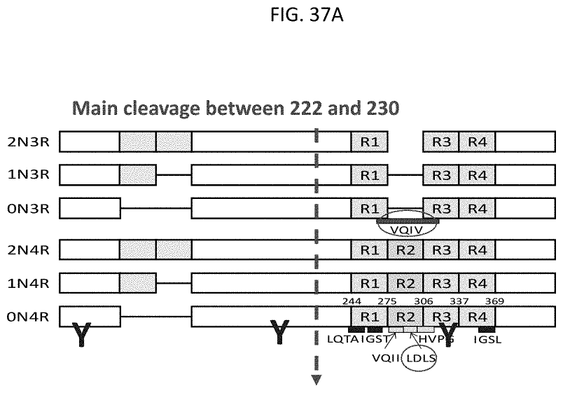

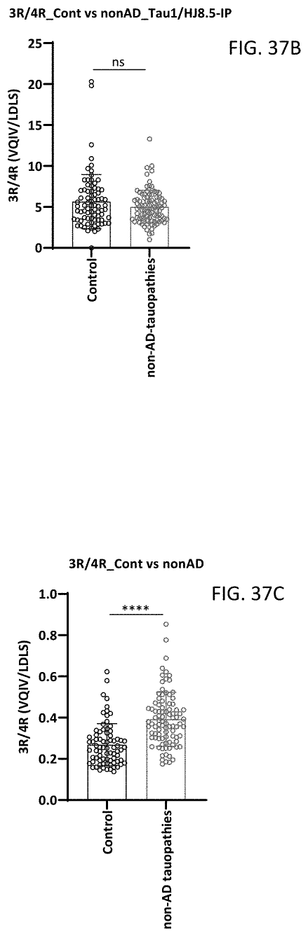

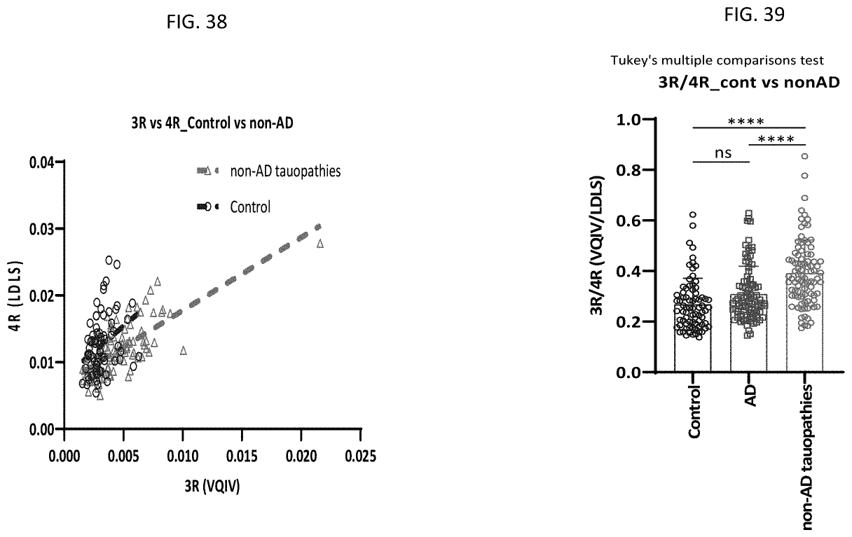

As shown in FIG. 37, the ratio of the tryptic peptides VQIV/LDLS in samples discriminates non-AD tauopathies from controls in samples processed by the PostIP-IP method (FIG. 37C) but not the IP method (FIG. 37B). Further analysis of the samples processed by the PostIP-IP method indicates a lower abundance of LDLS in CSF from subjects with non-AD tauopathies compared to control subjects (FIG. 38).

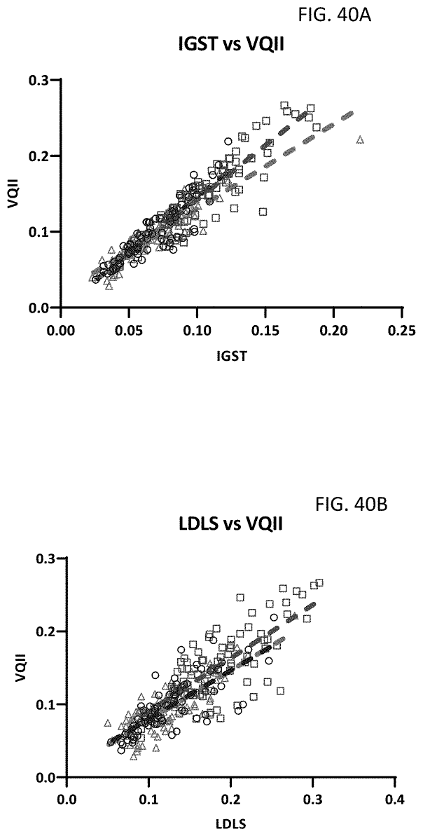

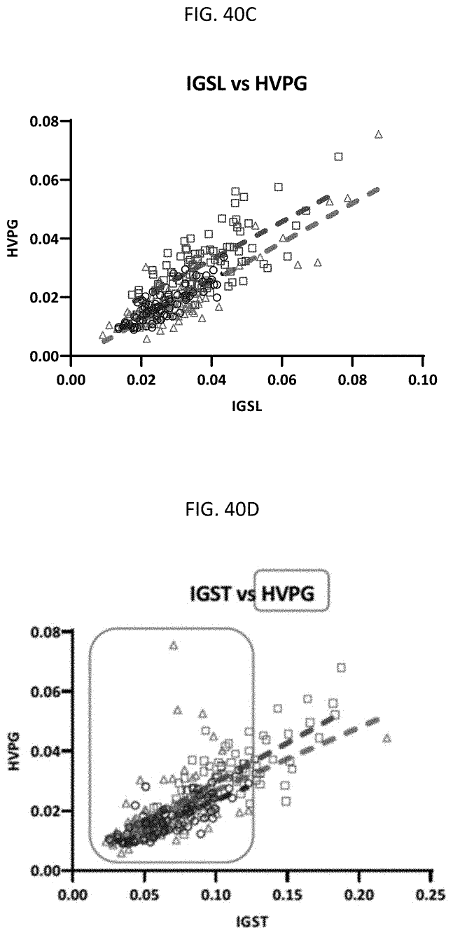

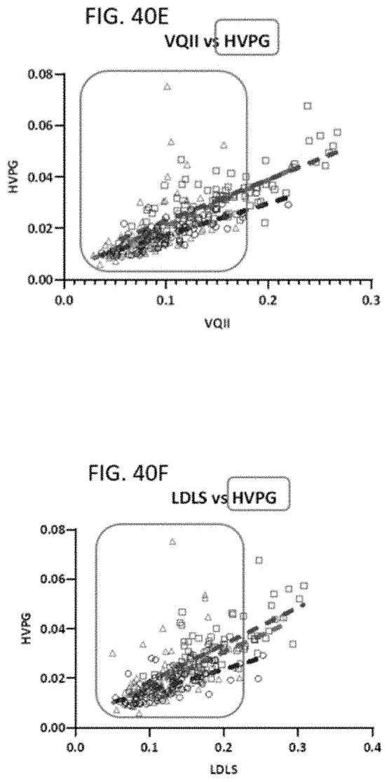

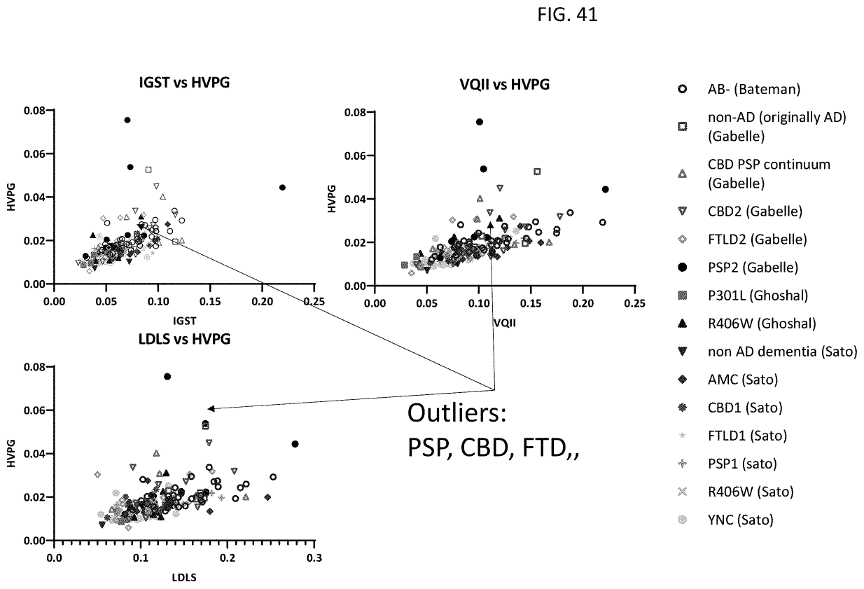

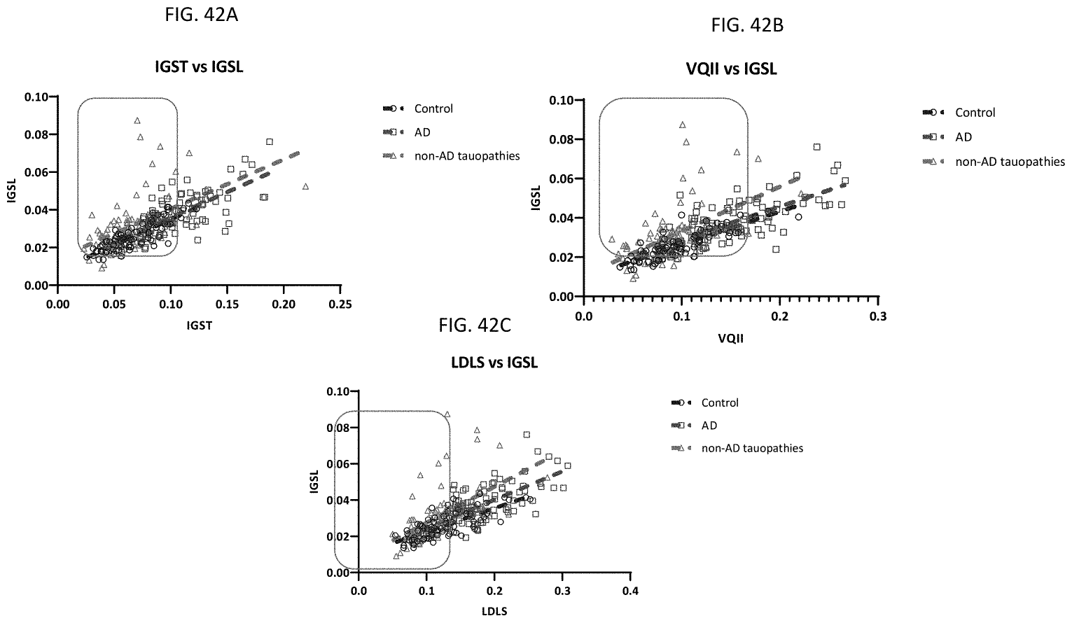

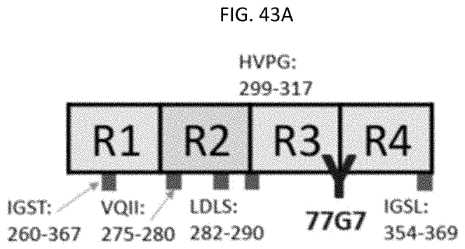

An increase in the VQIV/LDLS ratio was measured in PostIP-IP processed samples obtained from subjects with non-AD tauopathies, not in samples obtained from subjects with AD or from control subjects (FIG. 39). Analysis of additional tryptic peptides after PostIP-IP sample processing identified a lower correlation between R1-R2 tryptic peptides (e.g., IGST, VQII, LDLS, etc.) and later R2-R3 tryptic peptides (e.g., HVPG, etc.) in subjects with non-AD tauopathies as compared to subjects with AD or control subjects (FIG. 40, Table 8). Comparing among the non-AD tauopathies, certain subjects with PSP, CBD and FTD were outliers (FIG. 41). Similar results were obtained with comparisons to the tryptic peptide IGSL rather than HPVG (FIG. 42).

Overall, these data suggest that CSF tau profile measured after PostIP-IP sample processing could reflect brain tau aggregate status. For instance, 4R-tauopathies contain brain insoluble tau enriching R2 region, which comprises the VQII tryptic peptide, and some CSF samples from subjects with 4R-tauopathies showed reduced amounts of the tryptic peptides IGST, VQII, and LDLS relative to HVPG or IGSL. This was not observed in subjects with AD. In view of these data, methods to discriminate 4R-tauopathies should focus on enriching for the R2 region of MTBR tau.

TABLE 8

| | | IGST vs. | LDLS vs. | IGST vs. | LDLS vs. | VQII vs. | IGSL vs. |
|---|---|---|---|---|---|---|---|
| | | VQII | VQII | HVPG | HVPG | HVPG | HVPG |
| SLOPE | Control | 1.475 | 0.6411 | 0.1985 | 0.08943 | 0.1239 | 0.6411 |
| | AD | 1.43 | 0.6461 | 0.264 | 0.1499 | 0.1611 | 0.6461 |
| | Non-AD | 1.113 | 0.6661 | 0.21 | 0.1368 | 0.1793 | 0.6661 |
| | tauopathies | | | | | | |
| Pearson R | Control | 0.896 | 0.777 | 0.770 | 0.666 | 0.791 | 0.816 |
| | AD | 0.891 | 0.746 | 0.777 | 0.724 | 0.761 | 0.715 |
| | Non-AD | 0.915 | 0.815 | 0.523 | 0.496 | 0.543 | 0.854 |
| | tauopathies | | | | | | |

### Example 6

In this example, further analysis of the CSF and brain samples obtained from a single clinical cohort of subjects that included non-AD tauopathies provided additional evidence of the utility of mid-domain-independent MTBR tau to discriminate CSF samples obtained from subjects with non-AD tauopathies from CSF samples obtained from subjects with AD. Subjects in this cohort included subjects with AD (n=28), subjects clinically diagnosed with CBD or CBD/PSP (n=20), subjects clinically diagnosed with FTD (n=22), and subjects clinically diagnosed with PSP (n=11). CSF samples obtained from these subjects were processed by the PostIP-IP method as generally described in Example 4 and evaluated by mass spectrometry as generally described in Example 3. Brain insoluble tau was evaluated as described in Example 3.

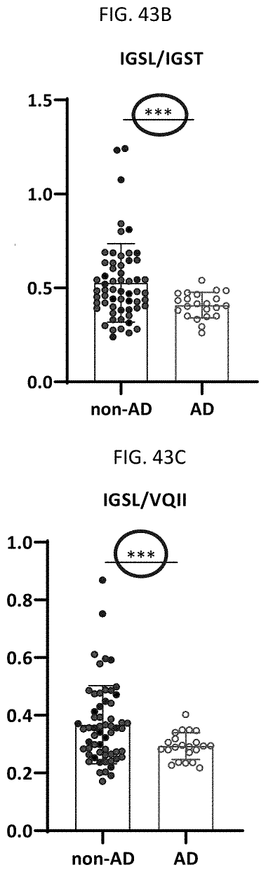

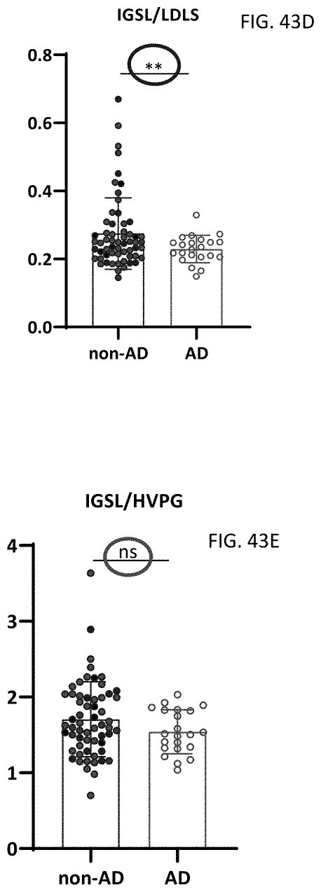

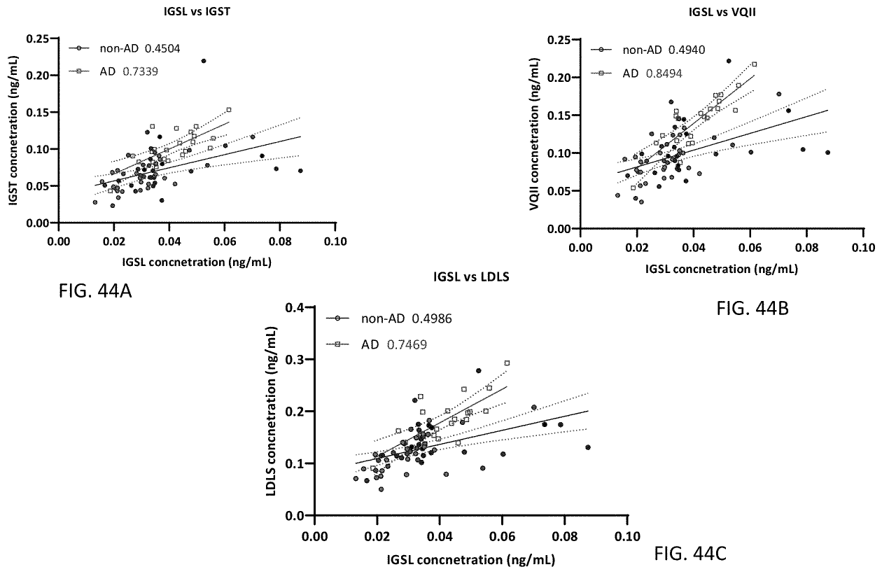

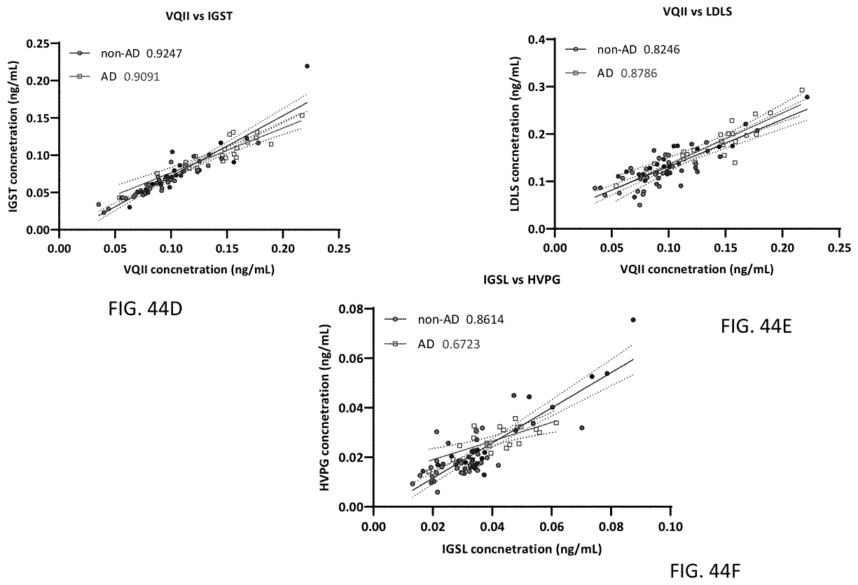

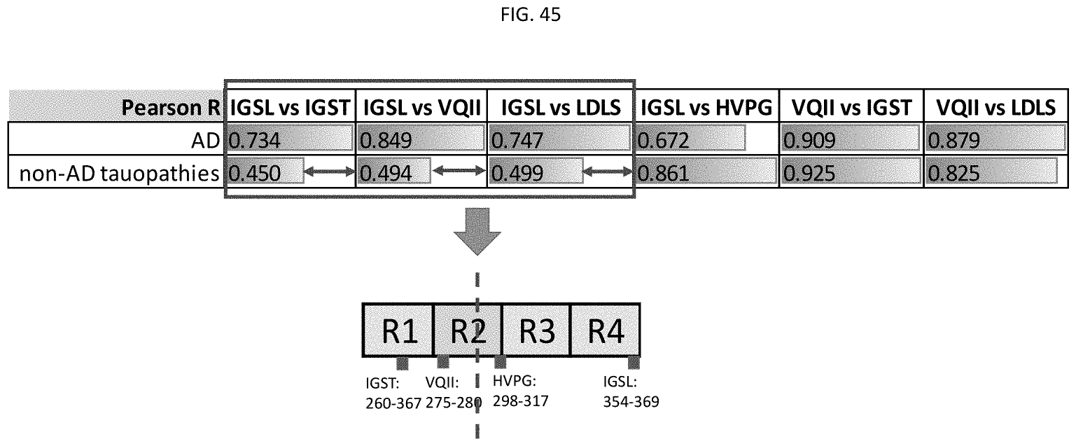

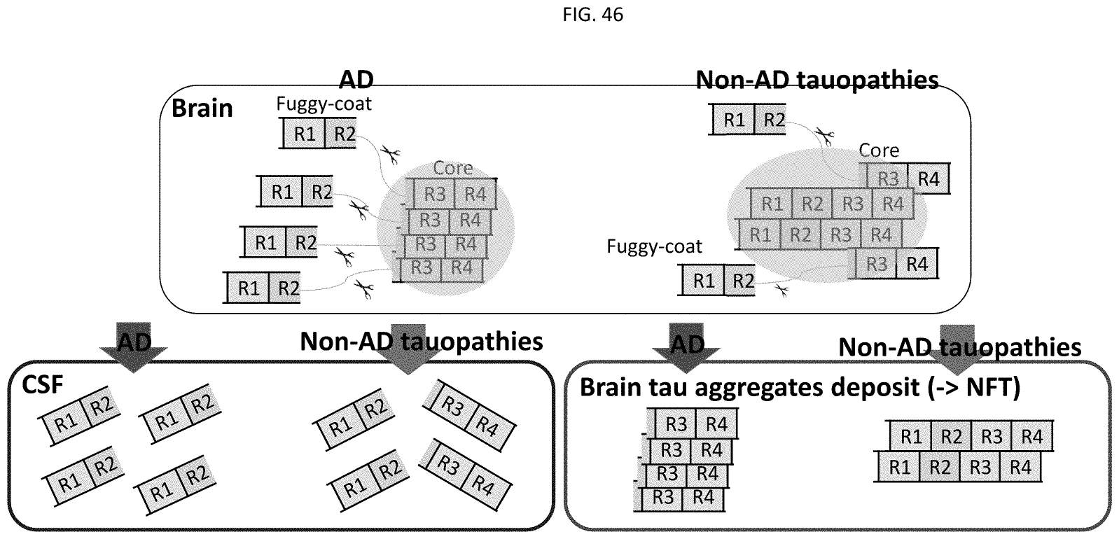

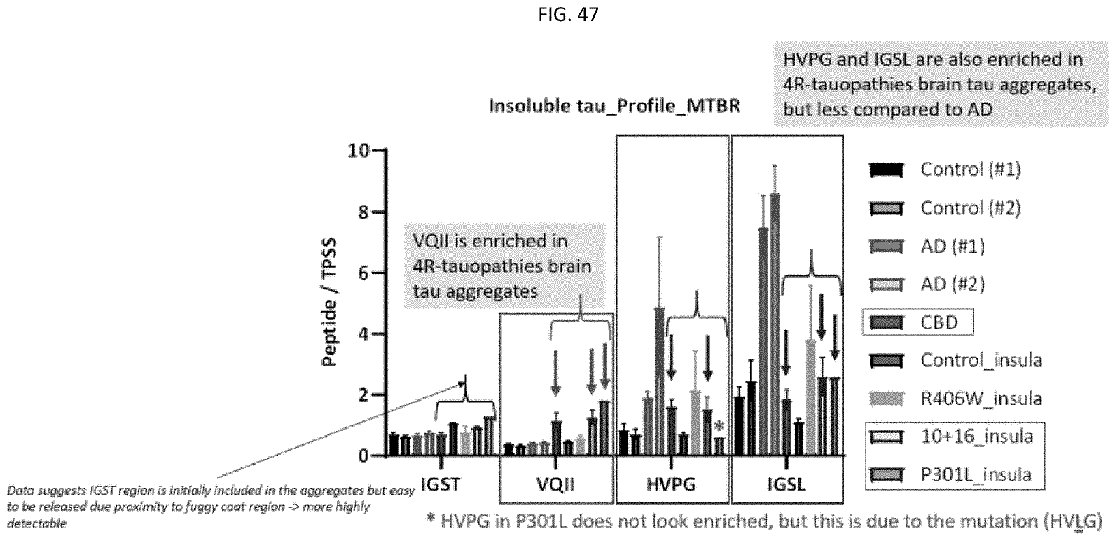

As noted in Example 5, analysis of tryptic peptides after PostIP-IP sample processing of CSF identified a low correlation between R1 and early R2 tryptic peptides (e.g., IGST, VQII, LDLS, etc.) and later R2, R3 and/or R4 tryptic peptides (e.g., IGSL, etc.) in subjects with non-AD tauopathies (FIG. 43, FIG. 44, and FIG. 45). It was hypothesized that in the CSF, tau species of non-AD tauopathies contain (1) less R1 and R2, and (2) more R3 and R4 than tau species of AD, and that this is a reflection of brain tau deposition (FIG. 46). To test this hypothesis, brain insoluble tau was analyzed. As shown in FIG. 47, the tryptic peptide VQII is enriched in brain tau aggregates of 4R-tauopathies. The tryptic peptides HVPG and IGSL are also enriched in brain tau aggregates but less compared to AD. These data further support the use of the ratios of R1 or R2 to R3 or R4 as a way to discriminate non-AD tauopathies from AD.

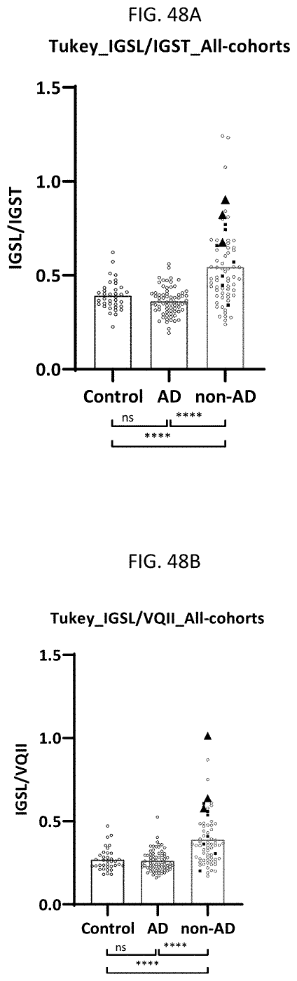

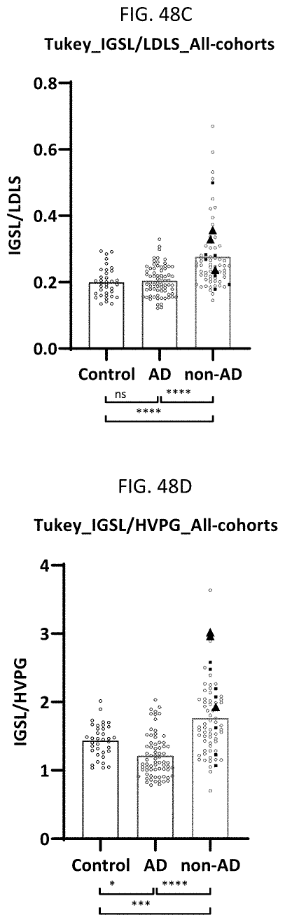

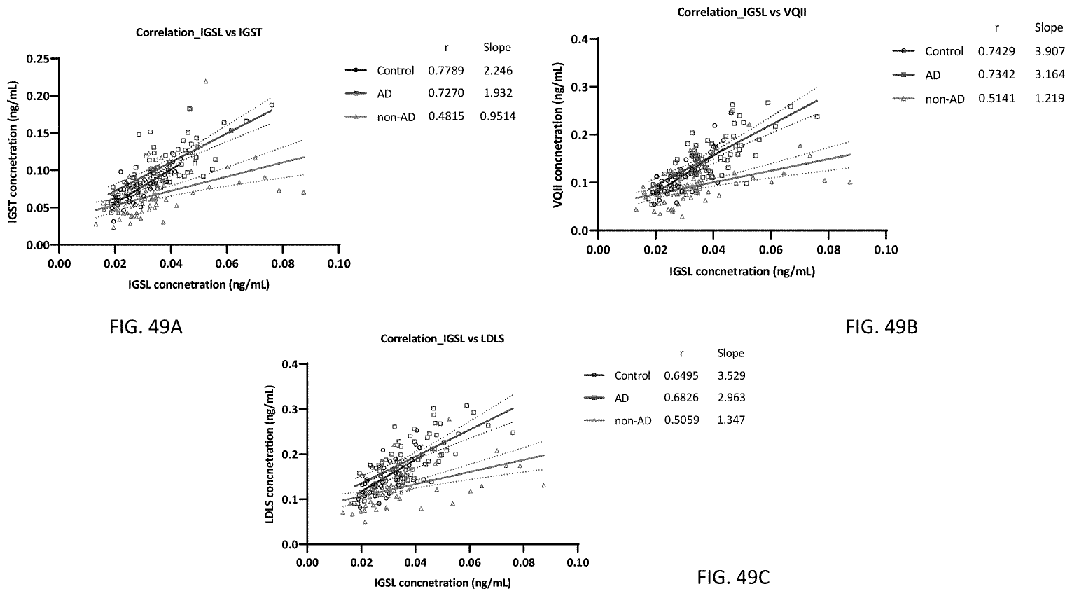

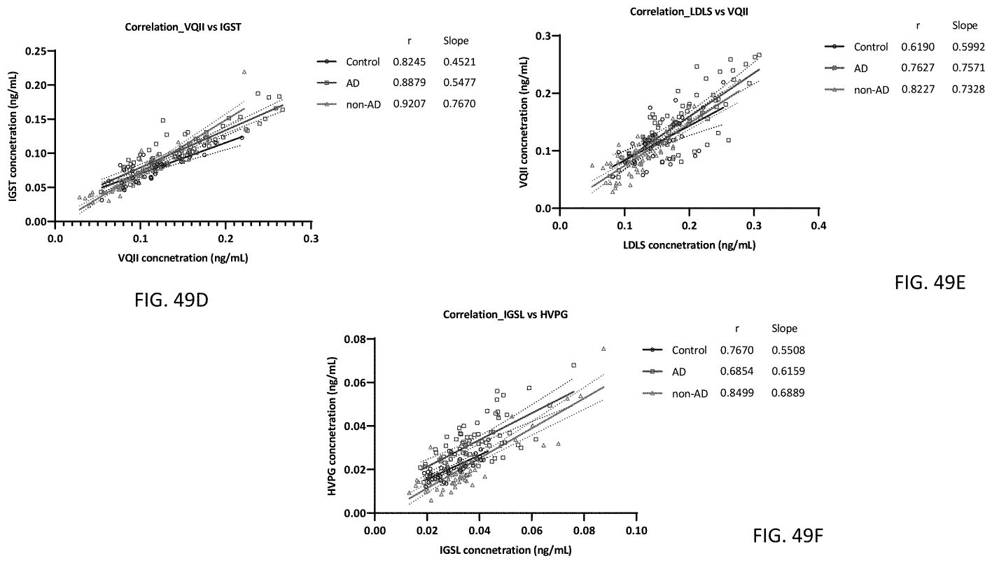

The analysis was then expanded to include CSF samples obtained from genetically confirmed FTLD cases (R406W, n=7; P301 L, n=3), CSF samples obtained from control subjects (n=44), and additional CSF samples from subjects with AD (n=41). CSF samples obtained from these subjects were processed by the PostIP-IP method as generally described in Example 4 and evaluated by mass spectrometry as generally described in Example 3. Analysis of tryptic peptides after PostIP-IP sample processing of CSF again identified a low correlation between R1 and early R2 tryptic peptides (e.g., IGST, VQII, LDLS, etc.) and later R2, R3 and/or R4 tryptic peptides (e.g., IGSL, etc.) in subjects with non-AD tauopathies (FIG. 48 and FIG. 49). These data confirm the use of the ratios of the amounts of R1 or R2 to R3 or R4 as a way to discriminate non-AD tauopathies from AD and also demonstrate the ability to discriminate control subjects from non-AD tauopathies. Notably, the use of CSF samples obtained from genetically confirmed FTLD cases provides further rigor to the analysis.

What is claimed is:

1. A method for measuring tau in a biological sample, the method comprising
(a) providing a biological sample selected from a blood sample or a CSF sample, wherein the biological sample (i) optionally comprises an isotope labeled internal standard of tau, and (ii) optionally is depleted of amyloid beta, N-terminal tau, mid-domain tau, or any combination thereof;
(b) removing proteins from the biological sample by protein precipitation and separation of the precipitated proteins to obtain a supernatant;
(c) purifying tau from the supernatant by solid phase extraction;
(d) cleaving the purified tau with a protease and then optionally desalting the resultant cleavage product by solid phase extraction to obtain a sample comprising proteolytic peptides of tau; and
(e) performing liquid chromatography-mass spectrometry with the sample comprising proteolytic peptides of tau to detect and measure the amount of at least one proteolytic peptide of tau.

2. The method of claim 1, wherein the biological sample is depleted of amyloid beta, N-terminal tau, mid-domain tau, or any combination thereof.

3. The method of claim 2, wherein (i) the biological sample is depleted of amyloid beta, N-terminal tau, and mid-domain tau, (ii) the biological sample is depleted of N-terminal tau and mid-domain tau, or (iii) the biological sample is depleted of mid-domain tau.

4. The method of claim 1, wherein the solid phase in step (c) comprises a reversed-phase sorbent that adsorbs tau.

5. The method of any one of the preceding claims, wherein:
step (b) comprises admixing an acid to precipitate proteins of the biological sample, optionally wherein the acid is perchloric acid; and/or
in step (e), the liquid chromatography-mass spectrometry is performed by a nano-LC/MS system.

6. A method for measuring tau in a biological sample, the method comprising
(a) decreasing in a biological sample by affinity depletion N-terminal tau, mid-domain tau, or N-terminal tau and mid-domain tau, and optionally decreasing by affinity depletion amyloid beta, wherein the biological sample is a blood sample or a CSF sample and the biological sample optionally comprises an isotope-labeled, tau internal standard;
(b) enriching MTBR tau by a method that comprises (i) removing additional proteins from the biological sample by protein precipitation and separation of the precipitated proteins to obtain a supernatant, and then purifying tau from the supernatant by solid phase extraction, or (ii) affinity purifying MTBR tau, thereby producing by either (i) or (ii) enriched MTBR tau;
(c) cleaving the enriched MTBR tau with a protease and then optionally desalting the resultant cleavage product by solid phase extraction to obtain a sample comprising proteolytic peptides of tau; and
(d) performing liquid chromatography-mass spectrometry (LC/MS) of the sample comprising proteolytic peptides of tau to detect and measure the amount of at least one proteolytic peptide of tau.

7. The method of claim 6, wherein step (a) comprises decreasing (i) N-terminal tau, mid-domain tau, or N-terminal tau and mid-domain tau, and (ii) amyloid beta.

8. The method of claim 6, wherein the biological sample comprises human tau and wherein step (a) further comprises
contacting the biological sample with at least one epitope-binding agent that specifically binds to an epitope within amino acids 1 to 243 of tau-441, inclusive; or
contacting the biological sample with a first epitope-binding agent that specifically binds to an epitope within amino acids 1 to 103 of tau-441, inclusive, and a second epitope-binding agent that specifically binds to an epitope within amino acids 104 to 243 of tau-441, inclusive; or
contacting the biological sample with a first epitope-binding agent that specifically binds to an epitope within amino acids 1 to 103 of tau-441, inclusive, a second epitope-binding agent that specifically binds to an epitope within amino acids 104 to 243 of tau-441, inclusive; and a third epitope binding agent that specifically binds to an epitope of amyloid beta; or
contacting the biological sample with a first epitope-binding agent that specifically binds to an epitope within amino acids 1 to 103 of tau-441, inclusive, a second epitope-binding agent that specifically binds to an epitope of amyloid beta; or
contacting the biological sample with a first epitope-binding agent that specifically binds to an epitope within amino acids 104 to 243 of tau-441, inclusive; and a third epitope binding agent that specifically binds to an epitope of amyloid beta.

9. The method of claim 8, wherein the epitope-binding agent that specifically binds to amyloid beta is HJ5.1.

10. The method of claim 8, wherein the epitope-binding agent that specifically binds to an epitope within amino acids 1 to 103 of tau-441, inclusive, is HJ8.5.

11. The method of claim 8, wherein the epitope-binding agent that specifically binds to an epitope within amino acids 104 to 243 of tau-441, inclusive, is Tau1.

12. The method of any one of claims 6 to 11, wherein solid phase extraction comprises a reversed-phase sorbent that adsorbs tau.

13. The method of any one of claims 6 to 11, wherein
the method for enriching MTBR tau comprises step (b)(i) and wherein the protein precipitation comprises admixing an acid to precipitate proteins of the biological sample, optionally wherein the acid is perchloric acid; and/or
in step (e), the liquid chromatography-mass spectrometry is performed by a nano-LC/MS system.

14. The method of claim 13, wherein the solid phase extraction performed in steps (b) and (c) comprises a reversed-phase sorbent that adsorbs tau.

15. The method of any one of claims 8 to 11, wherein
the method for enriching MTBR tau comprises step (b)(ii) and wherein affinity purifying MTBR tau comprises contacting the product of step (a) with an epitope-binding agent that specifically binds to an epitope that is C-terminal to the epitope of step (a); and/or
in step (e), the liquid chromatography-mass spectrometry is performed by a nano-LC/MS system.

16. The method of claim 15, wherein the epitope binding agent of step (b) specifically binds to an epitope
within amino acids 221 to 441 (inclusive) of tau-441, or
within amino acids 235 to 441 (inclusive) of tau-441, or
within amino acids 235 to 368 (inclusive) of tau-441, or
within amino acids 244 to 368 (inclusive) of tau-441, or
within amino acids 244 to 299 (inclusive) of tau-441.

17. The method of claim 16, wherein the epitope-binding agent is an antibody selected from the group consisting of 77G7, RD3, RD4, UCB0107, PT76, E2814 and 7G6.

18. The method of claim 15, 16 or 17, wherein the solid phase extraction performed in step (c) comprises a reversed-phase sorbent that adsorbs tau.

19. The method of any one of claims 1 to 5, wherein the protease is trypsin.

20. The method of claim 19, wherein step (d) comprises detecting and measuring the amount of at least one proteolytic peptide of tau, wherein the proteolytic peptide of tau has an amino acid sequence chosen from SEQ ID NO: 2, SEQ ID NO: 4, SEQ ID NO: 3, SEQ ID NO: 5, SEQ ID NO: 6, SEQ ID NO: 7, and SEQ ID NO: 9.

21. The method of claim 19, wherein step (d) comprises detecting and measuring the concentration of at least two proteolytic peptides of tau, wherein the two proteolytic peptides of tau have amino acid sequences chosen from SEQ ID NO: 2, SEQ ID NO: 4, SEQ ID NO: 3, SEQ ID NO: 5, SEQ ID NO: 6, SEQ ID NO: 7, SEQ ID NO: 8, and SEQ ID NO: 9.

22. The method of claim 21, wherein the at least two proteolytic peptides of tau have the amino acid sequence of SEQ ID NO: 6 and SEQ ID NO: 7, SEQ ID NO: 6 and SEQ ID NO: 8, SEQ ID NO: 3 and SEQ ID NO: 6, SEQ ID NO: 3 and SEQ ID NO: 7, SEQ ID NO: 3 and SEQ ID NO: 8, SEQ ID NO: 2 and SEQ ID NO: 7, SEQ ID NO: 2 and SEQ ID NO: 8, SEQ ID NO: 4 and SEQ ID NO: 7, SEQ ID NO: 4 and SEQ ID NO: 8, SEQ ID NO: 5 and SEQ ID NO: 7, SEQ ID NO: 5 and SEQ ID NO: 8, SEQ ID NO: 2 and SEQ ID NO: 6, SEQ ID NO: 4 and SEQ ID NO: 6, SEQ ID NO: 5 and SEQ ID NO: 6, or SEQ ID NO: 9 and SEQ ID NO: 5.

23. The method of any one of claims 6 to 11, wherein the protease is trypsin.

24. The method of claim 23, wherein step (d) comprises detecting and measuring the concentration at least one proteolytic peptide of MTBR tau, wherein the proteolytic peptide of tau has an amino acid sequence chosen from SEQ ID NO: 2, SEQ ID NO: 4, SEQ ID NO: 3, SEQ ID NO: 5, SEQ ID NO: 6, SEQ ID NO: 7, SEQ ID NO: 8, and SEQ ID NO: 9.

25. The method of claim 23, wherein step (d) comprises detecting and measuring the concentration of at least two proteolytic peptides of MTBR tau, wherein the two proteolytic peptides of tau have amino acid sequences chosen from SEQ ID NO: 2, SEQ ID NO: 4, SEQ ID NO: 3, SEQ ID NO: 5, SEQ ID NO: 6, SEQ ID NO: 7, SEQ ID NO: 8, and SEQ ID NO: 9.

26. The method of claim 25, wherein the at least two proteolytic peptides of MTBR tau have the amino acid sequence of SEQ ID NO: 6 and SEQ ID NO: 7, SEQ ID NO: 6 and SEQ ID NO: 8, SEQ ID NO: 3 and SEQ ID NO: 6, SEQ ID NO: 3 and SEQ ID NO: 7, SEQ ID NO: 3 and SEQ ID NO: 8, SEQ ID NO: 2 and SEQ ID NO: 7, SEQ ID NO: 2 and SEQ ID NO: 8, SEQ ID NO: 4 and SEQ ID NO: 7, SEQ ID NO: 4 and SEQ ID NO: 8, SEQ ID NO: 5 and SEQ ID NO: 7, SEQ ID NO: 5 and SEQ ID NO: 8, SEQ ID NO: 2 and SEQ ID NO: 6, SEQ ID NO: 4 and SEQ ID NO: 6, SEQ ID NO: 5 and SEQ ID NO: 6, and SEQ ID NO: 9 and SEQ ID NO: 5.

27. The method of claim 12, wherein the protease is trypsin.

28. The method of claim 13, wherein the protease is trypsin.

29. The method of claim 15, wherein the protease is trypsin.

30. The method of any one of claims 27, 28, or 29, wherein step (d) comprises detecting and measuring the concentration at least one proteolytic peptide of MTBR tau, wherein the proteolytic peptide of tau has an amino acid sequence chosen from SEQ ID NO: 2, SEQ ID NO: 4, SEQ ID NO: 3, SEQ ID NO: 5, SEQ ID NO: 6, SEQ ID NO: 7, SEQ ID NO: 8, and SEQ ID NO: 9.

31. The method of any one of claims 27, 28, or 29, wherein step (d) comprises detecting and measuring the concentration of at least two proteolytic peptides of MTBR tau, wherein the proteolytic peptides of tau have amino acid sequences chosen from SEQ ID NO: 2, SEQ ID NO: 4, SEQ ID NO: 3, SEQ ID NO: 5, SEQ ID NO: 6, SEQ ID NO: 7, SEQ ID NO: 8, and SEQ ID NO: 9.

32. The method of claim 31, wherein the at least two proteolytic peptides of MTBR tau have the amino acid sequence of SEQ ID NO: 6 and SEQ ID NO: 7, SEQ ID NO: 6 and SEQ ID NO: 8, SEQ ID NO: 3 and SEQ ID NO: 7, SEQ ID NO: 3 and SEQ ID NO: 8, SEQ ID NO: 2 and SEQ ID NO: 7, SEQ ID NO: 2 and SEQ ID NO: 8, SEQ ID NO: 4 and SEQ ID NO: 7, SEQ ID NO: 4 and SEQ ID NO: 8, SEQ ID NO: 5 and SEQ ID NO: 7, SEQ ID NO: 5 and SEQ ID NO: 8, SEQ ID NO: 2 and SEQ ID NO: 6, SEQ ID NO: 4 and SEQ ID NO: 6, SEQ ID NO: 5 and SEQ ID NO: 6, and SEQ ID NO: 9 and SEQ ID NO: 5.

33. The method of any one claims 6 to 32, wherein the method further comprises detecting and measuring the concentration of N-terminal tau, mid-domain tau, or amyloid beta removed from the biological sample in step (a).

34. A method for measuring Alzheimer disease (AD)-related pathology in a subject, the method comprising
providing a processed CSF or blood sample obtained from a subject, wherein the CSF or blood sample is depleted of mid-domain tau and enriched for MTBR tau; and
quantifying, in the processed sample, MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof, wherein the amount of the quantified MTRB-tau species or their ratios is a representation of AD-related pathology in a brain of a subject.

35. A method for measuring Alzheimer disease (AD)-related pathology in a subject, the method comprising measuring tau in a CSF or blood sample according to a method of any one of claims 6 to 18,
wherein the tau measured are MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof, wherein the amount of the measured MTRB-tau species or their ratios is a representation of AD-related pathology in a brain of a subject.

36. The method of claim 34 or 35, wherein the AD-related pathology is tau deposition in the subject's brain.

37. The method of claim 34 or 35, wherein the AD-related pathology is amyloid beta deposition in the subject's brain or brain arteries.

38. A method for measuring Alzheimer disease (AD)-related tau deposition in a brain of a subject, the method comprising
providing a processed CSF or blood sample obtained from a subject, wherein the processed CSF or blood sample is depleted of mid-domain tau and enriched for MTBR tau; and
quantifying, in the processed sample, MTBR tau species comprising the amino sequence of SEQ ID NO: 3 (LQTAPVPMPDLK) in the processed CSF or blood sample, wherein the amount of the quantified MTRB-tau species is a representation of AD-related tau deposition in a brain of a subject.

39. A method for measuring Alzheimer disease (AD)-related tau deposition in a brain of a subject, the method comprising measuring tau in a CSF or blood sample according to a method of any one of claims 6 to 18,
wherein the tau measured are MTBR tau species comprising the amino sequence of SEQ ID NO: 3 (LQTAPVPMPDLK), and wherein the amount of the measured MTRB-tau species is a representation of AD-related pathology in a brain of a subject.

40. A method for measuring Alzheimer disease (AD)-related tau deposition in a brain of a subject, the method comprising
providing a processed CSF or blood sample obtained from a subject, wherein the CSF or blood sample is depleted of mid-domain tau and enriched for MTBR tau; and
quantifying, in the processed sample, MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8), or a combination thereof, wherein the amount of the quantified MTRB-tau species or their ratios is a representation of AD-related tau deposition in a brain of a subject.

41. A method for measuring Alzheimer disease (AD)-related tau deposition in a brain of a subject, the method comprising measuring tau in a CSF or blood sample according to a method of any one of claims 6 to 18,
wherein the tau measured are MTBR tau species comprising the amino sequence of SEQ ID NO: 3, MTBR tau species comprising the amino sequence OF SEQ ID NO: 6, MTBR tau species comprising the amino sequence of SEQ ID NO: 7, MTBR tau species comprising the amino sequence of SEQ ID NO: 8, or a combination thereof, and wherein the amount of the measured MTRB-tau species or their ratios is a representation of AD-related pathology in a brain of a subject.

42. A method for diagnosing Alzheimer's disease, the method comprising
providing a processed CSF or blood sample obtained from a subject, wherein the CSF or blood sample is depleted of mid-domain tau and enriched for MTBR tau; and
quantifying, in the processed sample, MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof; and
diagnosing Alzheimer's disease when the quantified MTBR tau species differs by about 1.5σ or more, where σ is the standard deviation defined by the normal distribution measured in a control population that is amyloid negative as measured by PET imaging and/or Aβ42/40 measurement in CSF.

43. A method for diagnosing Alzheimer's disease, the method comprising
measuring tau in a CSF or blood sample according to a method of any one of claims 6 to 18, wherein the tau measured are MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof; and
diagnosing Alzheimer's disease when the quantified MTBR tau species differs by about 1.5σ or more, where σ is the standard deviation defined by the normal distribution measured in a control population that is amyloid negative as measured by PET imaging and/or Aβ42/40 measurement in CSF.

44. A method for measuring Alzheimer disease (AD) progression in a subject, the method comprising
providing a first processed CSF or blood sample and a second processed CSF or blood sample, wherein each processed sample is obtained from a single subject, and each processed sample is depleted of mid-domain tau and enriched for MTBR tau; and
for each processed sample, quantifying MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof; and
calculating the difference between the quantified MTBR tau species in the second sample and the first sample, wherein a statistically significant increase in the quantified MTBR tau species in the second sample indicates progression of the subject's Alzheimer's disease.

45. A method for measuring Alzheimer disease (AD) progression in a subject, the method comprising
measuring tau in a first and a second CSF or blood sample according to a method of any one of claims 6 to 18, wherein the tau measured are MTBR tau species comprising the amino sequence of SEQ ID NO: 3, MTBR tau species comprising the amino sequence of SEQ ID NO: 6, MTBR tau species comprising the amino sequence of SEQ ID NO: 7, MTBR tau species comprising the amino sequence of SEQ ID NO: 8, or a combination thereof; and
calculating the difference between the quantified MTBR tau species in the second sample and the first sample, wherein a statistically significant increase in the quantified MTBR tau species in the second sample indicates progression of the subject's Alzheimer's disease.

46. The method of any one of claims 38 to 45, wherein the subject is amyloid negative.

47. The method of claim 46, wherein the subject has no dementia.

48. The method of claim 46, wherein the subject has dementia.

49. The method of any one of claims 38 to 45, wherein the subject is amyloid positive.

50. The method of claim 49, wherein the subject has no dementia.

51. The method of claim 49, wherein the subject has dementia.

52. The method of claim 46 or claim 49, wherein the subject has a CDR score of 0.5 to 1.0.

53. The method of claim 46 or claim 49, wherein the subject has a CDR score of >1.0 to 2.0 (moderate AD).

54. The method of claim 46 or claim 49, wherein the subject has a CDR score of >2.0.

55. The method of any one of claims 38 to 54, the method further comprising quantifying amyloid beta, quantifying N-terminal tau, quantifying mid-domain tau, quantifying post-translational modifications of tau, or identifying an ApoE isoform in the biological or CSF sample.

56. A method for measuring tau pathology in a brain of a subject, the method comprising
providing a processed CSF or blood sample obtained from a subject, wherein the CSF or blood sample is depleted of mid-domain tau and enriched for MTBR tau; and
quantifying, in the processed sample, MTBR tau species comprising the amino acid sequence of SEQ ID NO: 2, MTBR tau species comprising the amino sequence of SEQ ID NO: 3, MTBR tau species comprising the amino sequence of SEQ ID NO: 4, MTBR tau species comprising the amino sequence of SEQ ID NO: 5, MTBR tau species comprising the amino acid sequence of SEQ ID NO: 6, MTBR tau species comprising the amino sequence of SEQ ID NO: 7, MTBR tau species comprising the amino sequence of SEQ ID NO: 8, MTBR tau species comprising the amino sequence of SEQ ID NO: 9, or combinations thereof, wherein the amount of the quantified MTRB-tau species or their ratios is a representation of tau pathology in a brain of a subject.

57. A method for measuring tau pathology in a brain of a subject, the method comprising measuring tau in a CSF or blood sample according to a method of any one of claims 6 to 18, wherein the tau measured are MTBR tau species comprising the amino acid sequence of SEQ ID NO: 2, MTBR tau species comprising the amino sequence of SEQ ID NO: 3, MTBR tau species comprising the amino sequence of SEQ ID NO: 4, MTBR tau species comprising the amino sequence of SEQ ID NO: 5, MTBR tau species comprising the amino acid sequence of SEQ ID NO: 6, MTBR tau species comprising the amino sequence of SEQ ID NO: 7, MTBR tau species comprising the amino sequence of SEQ ID NO: 8, MTBR tau species comprising the amino sequence of SEQ ID NO: 9, or combinations thereof, wherein the amount of the quantified MTRB-tau species or their ratios is a representation of tau pathology in a brain of a subject.

58. A method for discriminating a 4R-tauopathy, the method comprising
providing a processed CSF or blood sample obtained from a subject, wherein the CSF or blood sample is depleted of mid-domain tau and enriched for MTBR tau; and
quantifying, in the processed sample, (a) MTBR tau species comprising the amino sequence of SEQ ID NO: 9, and (b) MTBR tau species comprising the amino sequence of SEQ ID NO: 4, MTBR tau species comprising the amino sequence of SEQ ID NO: 5, MTBR tau species comprising the amino acid sequence of SEQ ID NO: 6, MTBR tau species comprising the amino sequence of SEQ ID NO: 7, or MTBR tau species comprising the amino sequence of SEQ ID NO: 8;
wherein the ratio of a quantified MTBR species from (a) to a quantified MTBR species from (b) discriminates a 4R-tauopathy.

59. A method for discriminating a 4R-tauopathy, the method comprising measuring tau in a biological sample according to a method of any one of claims 6 to 17, wherein the tau measured are (a) MTBR tau species comprising the amino sequence of SEQ ID NO: 9, and (b) MTBR tau species comprising the amino sequence of SEQ ID NO: 4, MTBR tau species comprising the amino sequence of SEQ ID NO: 5, MTBR tau species comprising the amino acid sequence of SEQ ID NO: 6, MTBR tau species comprising the amino sequence of SEQ ID NO: 7, or MTBR tau species comprising the amino sequence of SEQ ID NO: 8;
wherein the ratio of a quantified MTBR species from (a) to a quantified MTBR species from (b) discriminates a 4R-tauopathy.

60. A method for discriminating a 3R-tauopathy or a 4R-tauopathy from Alzheimer's disease, the method comprising
providing a processed CSF or blood sample obtained from a subject, wherein the CSF or blood sample is (a) depleted of mid-domain tau, and (b) enriched for MTBR tau, and
quantifying, in the processed sample, (a) MTBR tau species comprising the amino sequence of SEQ ID NO: 2, MTBR tau species comprising the amino sequence of SEQ ID NO: 4, MTBR tau species comprising the amino sequence of SEQ ID NO: 5, or combinations thereof, and (b) MTBR tau species comprising the amino acid sequence of SEQ ID NO: 6, MTBR tau species comprising the amino sequence of SEQ ID NO: 7, MTBR tau species comprising the amino sequence of SEQ ID NO: 8, or combinations thereof,
wherein the ratio of a quantified MTBR species from (a) to a quantified MTBR species from (b) discriminates a 3R-tauopathy or a 4R-tauopathy from Alzheimer's disease.

61. A method for discriminating a 3R-tauopathy or a 4R-tauopathy from Alzheimer's disease, the method comprising measuring tau in a biological sample according to a method of any one of claims 6 to 18, wherein the tau measured are (a) MTBR tau species comprising the amino sequence of SEQ ID NO: 2, MTBR tau species comprising the amino sequence of SEQ ID NO: 4, MTBR tau species comprising the amino sequence of SEQ ID NO: 5, or combinations thereof, and (b) MTBR tau species comprising the amino acid sequence of SEQ ID NO: 6, MTBR tau species comprising the amino sequence of SEQ ID NO: 7, MTBR tau species comprising the amino sequence of SEQ ID NO: 8, or combinations thereof,
wherein the ratio of a quantified MTBR species from (a) to a quantified MTBR species from (b) discriminates a 3R-tauopathy or a 4R-tauopathy from Alzheimer's disease.

62. A method for diagnosing a 4R-tauopathy, the method comprising
measuring tau in a CSF or blood sample according to a method of any one of claims 6 to 18, wherein the tau measured are (a) MTBR tau species comprising the amino sequence of SEQ ID NO: 2, MTBR tau species comprising the amino sequence of SEQ ID NO: 4, MTBR tau species comprising the amino sequence of SEQ ID NO: 5, or combinations thereof, and (b) MTBR tau species comprising the amino acid sequence of SEQ ID NO: 6), MTBR tau species comprising the amino sequence of SEQ ID NO: 7, MTBR tau species comprising the amino sequence of SEQ ID NO: 8, or combinations thereof; and
diagnosing a 4R-tauopathy when the quantified MTBR tau species differs by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF.

63. The method of any one of the claims 56 to 62, wherein the subject has no dementia.

64. The method of claim 63, wherein the 4R-tauopathy is corticobasal degeneration, Frontotemporal lobar degeneration, frontotemporal dementia, or progressive supranuclear palsy.

65. The method of any one of the claims 56 to 62, wherein the subject has dementia.

66. The method of claim 65, wherein the 4R-tauopathy is corticobasal degeneration, Frontotemporal lobar degeneration, frontotemporal dementia, or progressive supranuclear palsy.

67. A method for treating a subject in need thereof, the method comprising
providing a processed CSF or blood sample obtained from a subject, wherein the CSF or blood sample is (a) depleted of mid-domain tau, and (b) enriched for MTBR tau;
quantifying, in the processed sample, MTBR tau species comprising the amino acid sequence of SEQ ID NO: 2, MTBR tau species comprising the amino sequence of SEQ ID NO: 3, MTBR tau species comprising the amino sequence of SEQ ID NO: 4, MTBR tau species comprising the amino sequence of SEQ ID NO: 5, MTBR tau species comprising the amino acid sequence of SEQ ID NO: 6), MTBR tau species comprising the amino sequence of SEQ ID NO: 7, MTBR tau species comprising the amino sequence of SEQ ID NO: 8, MTBR tau species comprising the amino sequence of SEQ ID NO: 9, or combinations thereof; and
administering a treatment to the subject to alter tau pathology, wherein the subject's processed CSF or blood sample has quantified MTBR tau species, or ratios of the quantified MTBR tau species, that differ by about 1.5σ or more, where σ is the standard deviation defined by the normal distribution measured in a control population that does not have clinical signs or symptoms of a tauopathy and is amyloid negative as measured by PET imaging and/or Aβ42/40 measurement in CSF, and wherein the amount of the quantified MTRB-tau species or their ratios is a representation of tau pathology in a brain of a subject.

68. The method of claim 67, wherein the treatment alters or stabilizes the amount of the quantified MTBR species.

69. The method of claim 67 or 68, wherein the treatment is selected from the group consisting of cholinesterase inhibitors, N-methyl D-aspartate (NMDA) antagonists, antidepressants, gamma-secretase inhibitors, beta-secretase inhibitors, anti-Aβ antibodies, anti-tau antibodies, anti-TREM2 antibodies, TREM2 agonists, stem cells, dietary supplements, antagonists of the serotonin receptor 6, p38alpha MAPK inhibitors, recombinant granulocyte macrophage colony-stimulating factor, passive immunotherapies, active vaccines, tau protein aggregation inhibitors, therapies to improve blood sugar control, anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, and statins.

70. The method of claim 69, wherein the treatment is selected from the group consisting of anti-Aβ antibodies, anti-tau antibodies, anti-TREM2 antibodies, TREM2 agonists, gamma-secretase inhibitors, beta-secretase inhibitors, a kinase inhibitor, a phosphatase activator, a vaccine, and a tau protein aggregation inhibitor.

71. The method of claim 70, wherein the kinase inhibitor is an inhibitor of a thousand-and-one amino acid kinase (TAOK), CDK, GSK-3β, MARK, CDK5, or Fyn.

72. The method of claim 70, wherein the phosphatase activator increases the activity of protein phosphatase 2A.

73. The method of claim 70, wherein the vaccine is CAD106 or AF20513.

74. The method of claim 70, wherein the tau protein aggregation inhibitor is TRx0237 or methylthionimium chloride.

75. The method of claim 70, wherein the anti-Aβ antibody is aducanumab.
